(19) United States
(12) Patent Application Publication
(10) Pub. No.: US 2023/0372615 A1
(43) Pub. Date: Nov. 23, 2023

(54) SYSTEMS AND METHODS FOR DELIVERY OF MULTI-COMPONENT FLUIDS

(71) Applicant: TYBR Health, Inc., Houston, TX (US)

(72) Inventors: Peter Alexander SMITH, Houston, TX (US); Timothy J. KEANE, JR., Houston, TX (US); Stephen G. RAMON, Houston, TX (US); Kelvin Yaw Agyapong BOATENG, Houston, TX (US)

(21) Appl. No.: 18/324,584

(22) Filed: May 26, 2023

Related U.S. Application Data
(63) Continuation of application No. PCT/US2021/061216, filed on Nov. 30, 2021.
(60) Provisional application No. 63/119,548, filed on Nov. 30, 2020.

(51) Int. Cl. A61M 5/19; A61M 5/20

(52) U.S. Cl. CPC A61M 5/19; A61M 5/2066

(57) ABSTRACT

The disclosure provides a device and methods for delivering a multi-component fluid. In some cases, the device may comprise a tube, a mixer, and a nozzle. The tube may comprise a plurality of lumens carrying components of a multi-component fluid separately, and a dispersant. The mixer may receive and mix the separate multiple components to form the multi-component fluid. The dispersant may travel in a dispersant passageway disposed within the mixer. The multi-component fluid and the dispersant may be delivered through a nozzle outlet. In some cases, the dispersant and/or nozzle aerosolize the delivered multi-component fluid.

### CROSS-REFERENCE

This application is a continuation of PCT/US2021/061216, filed on Nov. 30, 2021, which claims the benefit of and priority to U.S. Provisional Application No. 63/119,548, filed Nov. 30, 2020, which are hereby incorporated by reference in their entirety herein.

### BACKGROUND

Surgical or clinical procedures sometimes have negative or unwanted outcomes. For example, internal scarring and fibrosis of the peritoneal, intrauterine, abdominal, joint, tendon, dermatologic, and other membranes may occur following surgical procedures, termed postoperative adhesions. Adhesions are considered frequent complication of abdominal surgery. Unlike other postoperative complications, such as wound infection or anastomotic leakage, the consequences of adhesion formation comprise a lifelong risk for various clinical entities. Adhesions may cause acute abdominal bowel obstruction, infertility, loss of range of motion, or chronic pain, other complications and patients may require reoperation or other medical intervention to treat resulting comorbidities.

### SUMMARY

Post-surgical adhesions present a significant to the subject following completion of surgery. For example, lysis of adhesions may be associated with a prolonged operative time in subsequent surgeries, increased dosages of anesthesia to complete the prolonged surgery, an increased risk of intraoperative complications such as hemorrhaging, postoperative complications such as nerve damage, or infertility, among other possible complications. Further, post-surgical adhesions present a significant burden upon the healthcare system at large due to the large number of adverse events resulting from adhesions associated comorbidities. Adhesions on the bowel are the number 1 cause of small bowel obstruction in the United States resulting in 400k emergency surgeries for intestinal obstruction repair procedures, with an estimated 300k of the 400k resulting from post-surgical adhesions. Presently, there are no reliable methods, devices or systems for preventing the formation of post-surgical adhesions. Responsive to this unmet need within the art, one or more embodiments of the present disclosure provides for devices, systems, and methods for preventing the formation of post-surgical adhesions in a subject.

One aspect of the present disclosure provides a device for delivering a multi-component fluid. The device may comprise a tube having a distal end and a proximal end, the tube comprising a first lumen, a second lumen, and a dispersant lumen, each lumen extending from the proximal end to the distal end of the tube. The first lumen may be configured to receive a first component of the multi-component fluid. The second lumen may be configured to receive a second component of the multi-component fluid. The dispersant lumen may be configured to receive a dispersant fluid. The device may further comprise a mixer coupled to the distal end of the tube, the mixer comprising a chamber disposed within a housing and a mixer body disposed within the chamber. A proximal end of the chamber may be in fluid communication with the first lumen and with the second lumen to receive the first component and the second component within the chamber and mix the first component and the second component using the mixer body to form the multi-component fluid. The mixer body may comprise a dispersant passageway therein that extends from a proximal end of the mixer body to a distal end of the mixer body and may be in fluid communication with the dispersant lumen to receive the dispersant fluid therefrom, so as to deliver the dispersant fluid to a distal end of the chamber or to a location distal to the distal end of the chamber. The device may also comprise a nozzle disposed distal to the mixer body and/or distal to the chamber. The nozzle may comprise a nozzle inlet, a nozzle body, and a nozzle outlet. The nozzle may receive the multi-component fluid from the chamber and the dispersant from the dispersant passageway, so as to deliver the multi-component fluid and the dispersant through the nozzle outlet.

In some embodiments, the dispersant passageway extends along a central axis of the mixer body from the mixer proximal end to the mixer distal end. In some embodiments, the dispersant passageway may be proximally coupled to the dispersant lumen. In some embodiments, the first lumen may be in fluid communication with a first container, such that the first lumen may be configured to receive the first component of the multi-component fluid from the first container. In some embodiments, the second lumen may be configured to receive the second component of the multi-component fluid from a second container. In some embodiments, the dispersant lumen may be configured to receive the dispersant fluid from a dispersant container. In some embodiments, the dispersant may be configured to receive the dispersant fluid from a pressurized source. In some embodiments, the dispersant fluid may comprise a compressed gas.

In some embodiments, the compressed gas may comprise oxygen, carbon dioxide, Nitrogen, helium, atmospheric air, argon, neon, xenon, krypton, radon, acetylene, butane, ethylene, hydrogen, methylamine, vinyl chloride, nitrogen oxides, halogen gases (e.g., chlorine, fluorine), acetylene,1,3-butadiene, methyl acetylene, tetrafluoroethylene, vinyl fluoride, or combinations thereof.

In some embodiments, a volumetric ratio between the first container and second container may be about 1:1 to about 5:1. In some embodiments, a volumetric ratio between the first container and second container may be about 1:1 to about 4:1. In some embodiments, i) the first container may comprise a first driver to deliver the first component from the first container to the first lumen, ii) the second container may comprise a second driver to deliver the second component from the second container to the second lumen, iii) the dispersant container may comprise a dispersant driver to deliver the dispersant from the dispersant container to the dispersant lumen, or iv) any combination thereof. In some embodiments, the first driver, the second driver, and/or the third driver may comprise a piston. In some embodiments, the first driver, the second driver, and/or the third driver may comprise a roller. In some embodiments, the first driver, the second driver, and/or the third driver may be driven manually. In some embodiments, the first driver, the second driver, and/or the third driver may comprise a plunger. In some embodiments, the first driver, the second driver, and/or the third driver may be driven by a compressed gas. In some embodiments, the first driver, the second driver, and/or the third driver may comprise a compressed gas. In some embodiments, the first driver, the second driver, and/or the third driver may be driven using an automatic actuation system. In some embodiments, the driver being driven refers to the driver being pushed down, or actuated. In some embodiments, the automatic actuation system may comprise a mechanical button, a pedal, or a digital button. In some embodiments, the first driver may be configured to deliver the first component to the first lumen and the second driver may be configured to deliver the second component to the second lumen at a constant volumetric ratio of the first component and the second component. In some embodiments, the volumetric ratio between the first container and second container may be about 1:1 to about 4:1. In some embodiments, a ratio between a volumetric rate of the first component entering the first lumen and a volumetric rate of the second component entering the second lumen may be constant or approximately constant. In some embodiments, the ratio of the volumetric rate between the first component entering the first lumen and the volumetric rate of the second component entering the second lumen may be about 1:1 to about 4:1. In some embodiments, a ratio of a cross-section between the first lumen and a cross-section of the second lumen may be about 1:1 to about 4:1. In some embodiments, the tube may comprise a third lumen configured to receive a third component of the multi-component fluid from a third container. In some embodiments, the tube may comprise four or more lumens. In some embodiments, the tube may be about 1 centimeter (cm) to about 100 cm long. In some embodiments, the tube may be about 1 millimeter (mm) to about 50 mm wide. In some embodiments, the mixer body may comprise a baffle, a blade, a channel, a slot, a plate, a fin, or a combination thereof. In some embodiments, the first lumen, the second lumen, and/or third lumen may be each in fluid communication with a respective fluid source (e.g., first container, second container, dispersant source) via a respective connector that may be coupled to the tube and respective fluid source.

In some embodiments, the mixer may be an inline mixer. In some embodiments, the mixer may be a static mixer. In some embodiments, the dispersant fluid aerosolizes the multi-component fluid i) prior to delivery through the nozzle outlet, ii) upon delivery through nozzle outlet, iii) after delivery from the nozzle outlet (e.g., distal to the nozzle outlet), or iv) combinations thereof. In some embodiments, the mixer body defines a dispersant passageway outlet at a distal end of the mixer body. In some embodiments, the dispersant passageway outlet may be disposed i) proximal of the nozzle outlet, ii) co-planar with the nozzle outlet (e.g., aligned with the nozzle outlet), or iii) distal to the nozzle outlet. In some embodiments, the dispersant passageway outlet has a diameter smaller than a diameter of the nozzle outlet. In some embodiments, the dispersant passageway outlet comprises an orifice. In some embodiments, the device further comprises a dispersant nozzle coupled to a distal end of the mixer body. The dispersant nozzle may comprise a dispersant nozzle outlet, wherein the dispersant nozzle may be in fluid communication with the dispersant passageway. In some embodiments, the dispersant nozzle extends from the mixer body and into the nozzle. In some embodiments, the dispersant nozzle outlet may be disposed i) proximal of the nozzle outlet, ii) co-planar with the nozzle outlet (e.g., aligned with the nozzle outlet), or iii) distal to the nozzle outlet. In some embodiments, the dispersant nozzle outlet has a diameter smaller than a diameter of the nozzle outlet. In some embodiments, the nozzle inlet has a diameter of about 0.1 millimeter (mm) to about 4 mm. In some embodiments, the nozzle outlet has a diameter of about 0.1 millimeter (mm) to about 4 mm. In some embodiments, the nozzle body may be about 0.1 millimeter (mm) to about 10 mm long. In some embodiments, the nozzle inlet diameter may be larger than the nozzle outlet diameter. In some embodiments, the nozzle inlet diameter may be smaller than the nozzle outlet diameter. In some embodiments, the nozzle may be tapered inward towards the nozzle outlet. In some embodiments, the nozzle may be tapered outward towards the nozzle outlet. In some embodiments, the nozzle may comprise a channel. In some embodiments, the nozzle may comprise a plurality of channels. In some embodiments, the nozzle may be disposed within the housing distal to mixer body. In some embodiments, the nozzle may be embedded within the housing distal to mixer body. In some embodiments, the nozzle may be coupled to a distal end of the housing. In some embodiments, the device further comprises a steering element to control an orientation of the mixer and the nozzle, so as to control a direction of delivering the multi-component fluid through the nozzle outlet. In some embodiments, the tube may comprise a flexible portion. In some embodiments, at least a portion of the tube may be configured to curve. In some embodiments, a distal portion of the tube may be configured to curve. In some embodiments, the steering element may be configured to control the curvature of a distal portion of the tube, thereby enabling control of the orientation of the mixer and nozzle. In some embodiments, the steering element may comprise a rigid tube. In some embodiments, the tube may be at least partially disposed within the rigid tube, such that at least a portion of the tube not disposed within the rigid tube may be curved. In some embodiments, the steering element may be configured to slide over the tube to apply a force on the curved portion of the tube. In some embodiments, the steering element may be configured to slide over the tube to straighten or nearly straighten at least part of the curved portion of the tube. In some embodiments, the steering element may comprise a surface coating to reduce mechanical friction as it slides over the tube. In some embodiments, the first component may comprise an extracellular (ECM) matrix.

In some embodiments, the second component may comprise a buffer solution. In some embodiments, the buffer solution may comprise a pH buffer solution (e.g., phosphate-buffered saline). In some embodiments, the multicomponent fluid delivered from the device may comprise a plurality of particles. In some embodiments, the particles have a diameter of about 10 μm to 500 μm. In some embodiments, the particles have a diameter of at most about 100 μm. In some embodiments, the plurality of particles may be formed at a distance distal to the nozzle outlet. In some embodiments, the distance may be at most 5 centimeters (cm) from the nozzle outlet. In some embodiments, the dispersant nozzle comprises an internal diameter of about 0.3 mm. In some embodiments, the dispersant nozzle comprises an internal diameter of about 0.6 mm. In some embodiments, the device may comprise a dispersant fluid, and the dispersant fluid may be CO2. In some embodiments, the dispersant fluid may be pressurized from about 5 psig to about 100 psig. In some embodiments, the dispersant fluid is pressurized at about 5 psig. In some embodiments, the dispersant fluid is pressurized at about 10 psig. In some embodiments, the device may be configured to aerosolize the multicomponent fluid upon dispersion. In some embodiments, the device may be configured to aerosolize the multicomponent fluid upon dispersion to a mist comprising particles, and the particles comprise an average particle diameter from about 7 um to about 300 um. In some embodiments, the device may be configured to aerosolize the multicomponent fluid upon dispersion to a mist comprising particles, and the particles comprise an average particle diameter of about 128 um. In some embodiments, the device may be configured to aerosolize the multicomponent fluid upon dispersion to a mist comprising particles, and the particles comprise an irregular particle diameter distribution, and the most frequent particle diameter may be between 7 um to about 136 um. In some embodiments, the device may be configured to aerosolize the multicomponent fluid upon dispersion to a mist comprising particles, comprises an average particle area of about 12,700 square um. In some embodiments, the device may be configured to aerosolize the multicomponent fluid upon dispersion to a mist comprising particles and the particles comprise an irregular particle area distribution, and the most frequent particle area may be from about 41 square um to about 10,000 square um. In some embodiments, the multi-component fluid comprises an extracellular (ECM) matrix, the device may be configured to aerosolize the multicomponent fluid upon dispersion to an ECM hydrogel mist comprising particles. In some embodiments, the device may be configured to produce an average particle diameter from about 7 um to about 300 um. In some embodiments, the device may be configured to produce an average particle diameter of about 128 um. In some embodiments, the particles comprise an irregular particle diameter distribution, and the most frequent particle diameter may be between 7 um to about 136 um. In some embodiments, the particles comprise an average particle area from about 41 square um to about 70,000 square um. In some embodiments, the particles comprise an average particle area of about 12,700 square um. In some embodiments, the device may be configured to produce an ECM hydrogel scaffold. The device of claim 121, the ECM hydrogel scaffold comprises a storage modulus of at least 1000 Pa within about 500 seconds of dispersing. In some embodiments, the ECM hydrogel scaffold comprises a storage modulus of at least 2500 Pa. In some embodiments, the ECM hydrogel scaffold comprises a storage modulus of at least 2500 Pa within about 750 seconds of dispersing. In some embodiments, the ECM hydrogel scaffold comprises a storage modulus of at least 6000 Pa within about 1000 seconds of dispersing. In some embodiments, device may be configured to produce an ECM hydrogel scaffold which is a substantially homogenous solution. In some embodiments, the distal end the tube may be flexible and elongated, the device further comprises a steering element configured to control the curvature of a distal portion of the tube, thereby enabling control of the orientation of the mixer and nozzle, and the device may be configured for use in laparoscopic surgery.

Another aspect of the present disclosure provides a method for delivering a multi-component fluid. The method may comprise providing a delivery device. The delivery device may comprise a tube having a distal end and a proximal end, the tube comprising a first lumen, a second lumen, and a dispersant lumen, each lumen extending from the proximal end to the distal end of the tube. The first lumen may be configured to receive a first component of the multi-component fluid. The second lumen may be configured to receive a second component of the multi-component fluid. The dispersant lumen may be configured to receive a dispersant fluid. The delivery device may further comprise a mixer coupled to the distal end of the tube, the mixer comprising a chamber disposed within a housing and a mixer body disposed within the chamber. A proximal end of the chamber may be in fluid communication with the first lumen and with the second lumen to receive the first component and the second component within the chamber and mix the first component and the second component using the mixer body to form the multi-component fluid. The mixer body may comprise a dispersant passageway therein that extends from a proximal end of the mixer body to a distal end of the mixer body and may be in fluid communication with the dispersant lumen to receive the dispersant fluid therefrom, so as to deliver the dispersant fluid to a distal end of the chamber or to a location distal to the distal end of the chamber. The delivery device may further comprise a nozzle disposed distal to the mixer body and/or distal to the chamber. The nozzle may comprise a nozzle inlet, a nozzle body, and a nozzle outlet. The nozzle receives the multi-component fluid from the chamber and the dispersant from the dispersant passageway, so as to deliver the multi-component fluid and the dispersant through the nozzle outlet. The method may further comprise delivering the first component and second component through the first lumen and second lumen respectively, such that the first component and second mix via the mixer to form the multi-component fluid. The first component may be delivered from a first component source. The second component may be delivered from a second component source. The method may further comprise delivering a dispersant through the dispersant lumen and the dispersant passageway, such that the dispersant fluid aerosolizes the multi-component fluid through interaction therewith within the nozzle, through the nozzle outlet, and/or after being delivered from the nozzle outlet. The method may further comprise controlling the orientation of the mixer and nozzle to control the direction of delivery of the multi-component fluid from the device. The method may further comprise any of the devices disclosed.

In some embodiments, the dispersant may be CO2. In some embodiments, the dispersant may be CO2 may be delivered from about 5 psig to about 100 psig. In some embodiments, the dispersant may be CO2 may be delivered at about 5 psig. In some embodiments, the dispersant may be CO2 may be delivered at about 10 psig. In some embodiments, the multi-component fluid comprises an extracellular (ECM) matrix. In some embodiments, the multi-component fluid may be aerosolized to an ECM hydrogel when dispersed through the dispersant lumen and the dispersant passageway, the crosslinked ECM hydrogel mist comprises a plurality of particles. In some embodiments, the particles comprise an average particle diameter from about 7 um to about 300 um. In some embodiments, the particles comprise an average particle diameter of about 128 um. In some embodiments, the particles comprise irregular particle diameter distribution, and the most frequent particle diameter may be between 7 um to about 136 um. In some embodiments, the particles comprise an average particle area from about 41 square um to about 70,000 square um. In some embodiments, the particles comprise an average particle area of about 12,700 square um. In some embodiments, the particles comprise irregular particle area distribution, and the most frequent particle area may be from about 41 square um to about 10,000 square um. In some embodiments, the multi-component fluid further comprises a buffer solution. In some embodiments, the buffer solution may be a phosphate buffered solution. In some embodiments, the multi-component may be buffered to a mildly acidic pH. In some embodiments, the multi-component may be buffered to a pH from about 6.5 to about 7.0. In some embodiments, the nozzle further comprises a gas orifice fluidically connected to a pressurized gas source. In some embodiments, the gas orifice comprises a 0.6 mm opening for dispersing the gas. In some embodiments, the nozzle further comprises an annular channel surrounding the gas orifice for passage of the multicomponent fluid. In some embodiments, the gas orifice may be flush with the nozzle outlet. In some embodiments, the gas orifice may be offset from the nozzle outlet in the distal direction. In some embodiments, the gas orifice may be offset from the nozzle outlet in the proximal direction. In some embodiments, the gas orifice comprises a 0.3 mm opening for dispersing the gas. In some embodiments, the crosslinked ECM hydrogel mist forms an ECM hydrogel scaffold comprising a storage modulus of at least 1500 Pa. The method of claim 88, further comprising dispersing the crosslinked ECM hydrogel mist from the delivery device. In some embodiments, dispersing the crosslinked ECM hydrogel mist forms an ECM hydrogel scaffold. In some embodiments, the ECM hydrogel scaffold comprises a storage modulus of at least 1000 Pa within about 500 seconds of dispersing. In some embodiments, the ECM hydrogel scaffold comprises a storage modulus of at least 2500 Pa. In some embodiments, the ECM hydrogel scaffold comprises a storage modulus of at least 2500 Pa within about 500 seconds of dispersing. In some embodiments, the ECM hydrogel scaffold comprises a storage modulus of at least 2500 Pa within about 750 seconds of dispersing. In some embodiments, the ECM hydrogel scaffold comprises a storage modulus of at least 6000 Pa within about 1000 seconds of dispersing. In some embodiments, the ECM hydrogel scaffold may be a substantially homogenous solution. In some embodiments, the multicomponent fluid may be a shear thinning fluid, the method further comprising reducing the viscosity of the multicomponent fluid upon mixing in the device or upon dispersion from the device. In some embodiments, the multicomponent fluid may be a shear thinning fluid, the method further comprising reducing the viscosity of the multicomponent fluid upon mixing in the device or upon dispersion from the device, further comprising coating a tissue with the multicomponent fluid, wherein the coating of the tissue of the multicomponent fluid may be expedited as a result of reducing the viscosity, and an even coat of the multicomponent fluid may be placed upon the tissue. In some embodiments, the tissue is in vivo.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

One or more aspects of the present disclosure provides a system comprising: a device for delivering a multicomponent fluid, the device comprising: a tube having a distal end and a proximal end, wherein the tube comprising a first lumen, a second lumen, and a dispersant lumen, each lumen extending from the proximal end to the distal end of the tube, wherein: the first lumen may be configured to receive a first component of the multi-component fluid, the second lumen may be configured to receive a second component of the multi-component fluid, and the dispersant lumen may be configured to receive a dispersant fluid; a mixer coupled to the distal end of the tube, the mixer comprising a chamber disposed within a housing and a mixer body disposed within the chamber, wherein a proximal end of the chamber may be in fluid communication with the first lumen and with the second lumen to receive the first component and the second component within the chamber and mix the first component and the second component using the mixer body to form the multi-component fluid, wherein the mixer body comprises a dispersant passageway therein that extends from a proximal end of the mixer body to a distal end of the mixer body and may be in fluid communication with the dispersant lumen to receive the dispersant fluid therefrom, so as to deliver the dispersant fluid to a distal end of the chamber or to a location distal to the distal end of the chamber; a nozzle disposed distal to the mixer body and/or distal to the chamber, wherein the nozzle comprises a nozzle inlet, a nozzle body, and a nozzle outlet, wherein the nozzle receives the multi-component fluid from the chamber and the dispersant from the dispersant passageway, so as to deliver the multi-component fluid and the dispersant through the nozzle outlet; a dispersant nozzle coupled to a distal end of the mixer body, wherein the dispersant nozzle comprises a dispersant nozzle outlet, wherein the dispersant nozzle may be in fluid communication with the dispersant passageway; and a multi component fluid in fluidic communication with the device, the multi component fluid comprising: an extracellular (ECM) matrix; a buffering solution, and a pressurized dispersant fluid in fluidic communication with the device.

In some embodiments, the pressurized dispersant fluid comprises CO2. In some embodiments, the buffering solution comprises phosphate-buffered saline. In some embodiments, the dispersant nozzle outlet may be between 0.3 and 0.9 mm in diameter. In some embodiments, the dispersant nozzle outlet may be 0.6 mm in diameter. In some embodiments, the dispersant fluid aerosolizes the multi-component fluid upon delivery from the nozzle outlet into an ECM hydrogel mist comprising particles. In some embodiments, the particles comprise an average particle diameter from about 7 um to about 300 um. In some embodiments, the particles comprise an average particle diameter of about 128 um. In some embodiments, the particles comprise an irregular particle diameter distribution, and the most frequent particle diameter may be between 7 um to about 136 um. In some embodiments, the particles comprise an average particle area from about 41 square um to about 70,000 square um. In some embodiments, the particles comprise an average particle area of about 12,700 square um. In some embodiments, the particles comprise irregular particle area distribution, and wherein the most frequent particle area may be from about 41 square um to about 10,000 square um. In some embodiments, the multi-component may be buffered to a mildly acidic pH. In some embodiments, the multi-component may be buffered to a pH from about 6.5 to about 7.0. In some embodiments, the multicomponent fluid comprises an ECM hydrogel scaffold. In some embodiments, the extracellular (ECM) matrix, the buffering solution, and the pressurized dispersant fluid collectively comprise an ECM hydrogel scaffold. In some embodiments, the ECM hydrogel scaffold comprises a storage modulus of at least 1000 Pa. In some embodiments, the ECM hydrogel scaffold comprises a storage modulus of at least 1000 Pa within about 500 seconds of dispersing. In some embodiments, the ECM hydrogel scaffold comprises a storage modulus of at least 2500 Pa. In some embodiments, the ECM hydrogel scaffold comprises a storage modulus of at least 2500 Pa within about 500 seconds of dispersing. In some embodiments, the ECM hydrogel scaffold comprises a storage modulus of at least 2500 Pa within about 750 seconds of dispersing. In some embodiments, the ECM hydrogel scaffold may be a substantially homogenous solution. In some embodiments, the particles comprise droplets. In some embodiments, the particles may be droplets. In some embodiments, the ECM may be acidic. In some embodiments, the ECM comprise a pH of about 1 to about 3. In some embodiments, the ECM form an ECM hydrogel scaffold when buffered to a mildly acidic, neutral, or mildly basic pH. In some embodiments, the ECM form an ECM hydrogel scaffold when buffered to a pH of between 6.0 and 7.5. In some embodiments, the ECM may be a shear thinning fluid. In some embodiments, the multicomponent fluid may be a shear thinning fluid.

One or more aspects of the present disclosure provides a system comprising: a device for delivering a fluid, the device comprising: a tube having a distal end and a proximal end, wherein the tube comprises a plurality of lumens, wherein: a first lumen of the plurality of lumens is configured to receive a first fluid, and a second lumen of the plurality of lumens is configured to receive a second fluid; a mixer coupled to the tube, the mixer comprising a chamber is in fluid communication at least one lumen to receive the first fluid, and a dispersant passageway therein that extends through the mixer and which is in fluid communication with the second lumen to receive the second fluid; and a nozzle disposed distal to the chamber, wherein the nozzle receives the first fluid from the mixer and the second fluid from the dispersant passageway, so as to deliver the first and second fluids through a nozzle outlet.

In some embodiments, a third lumen of the plurality of lumens is configured to receive a third fluid. In some embodiments, the mixer is configured mix to the first and the third fluid to a mixture. In some embodiments, the second fluid is a gas. In some embodiments, the nozzle is configured to disperse the mixture with the second fluid.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example of a spray device for multi-component fluids according to some embodiments.

FIG. 2 provides an example of a front view of a three-lumen tube according to some embodiments.

FIG. 3 provides another example of a front view of a five-lumen tube according to some embodiments.

FIG. 4A provides an example of a perspective view of an inline mixer and a nozzle according to some embodiments.

FIG. 4B provides an example of a side view of an inline mixer and a nozzle according to some embodiments.

FIG. 4C provides an example of a cross-sectional view of an inline mixer and a nozzle according to some embodiments.

FIG. 5A provides a perspective view of an exemplary mixer according to some embodiments.

FIG. 5B provides a side view of another exemplary mixer according to some embodiments.

FIG. 6A provides a perspective view of another mixer according to some embodiments.

FIG. 6B provides a side view of an exemplary mixer according to some embodiments.

FIG. 7A provides an example of a perspective view of a mixer with a combination of blades and baffles according to some embodiments.

FIG. 7B provides an example of a side view of a mixer with a combination of blades and baffles according to some embodiments.

FIG. 8A provides an example of a perspective view of an exemplary mixer according to some embodiments.

FIG. 8B provides an example of a side view of an exemplary mixer according to some embodiments.

FIG. 9 provides an example of a perspective view of a mixer with intersecting tubes according to some embodiments.

FIG. 10 provides an example of a cross-sectional view of a nozzle with circular outlet according to some embodiments.

FIG. 11 provides an example of a cross-sectional view of a nozzle with microtube features according to some embodiments.

FIG. 12A provides an example of a cross-sectional view of a nozzle with a geometric feature according to some embodiments.

FIG. 12B illustrates an example of a nozzle with plurality of small channels according to some embodiments.

FIG. 13 provides an example of a perspective view of an applicator tip according to some embodiments.

FIG. 14 schematically illustrates an example of aerosolization of a fluid using a spray device according to some embodiments.

FIG. 15A schematically illustrates an experimental set up to observe fluid exiting a nozzle according to some embodiments.

FIG. 15B provides an example of a syringe pump according to some embodiments.

FIG. 16 provides a schematic example of a nozzle according to some embodiments.

FIG. 17 provides a schematic example of a nozzle according to some embodiments.

FIG. 18 provides a schematic example of a nozzle according to some embodiments.

FIG. 19 provides a schematic example of a nozzle according to some embodiments.

FIG. 20 provides a schematic example of a nozzle according to some embodiments.

FIG. 21 provides a schematic example of a nozzle according to some embodiments.

FIG. 22 provides a schematic example of a nozzle according to some embodiments.

FIG. 23 provides a schematic example of a nozzle according to some embodiments.

FIG. 24A provides an image of an example of an aerosolized fluid of low viscosity exiting a nozzle according to some embodiments.

FIG. 24B provides an image of an example of a fluid jet from exiting a nozzle according to some embodiments.

FIG. 24C provides an image of an example of a droplet from a highly viscous fluid exiting a nozzle according to some embodiments.

FIG. 25 is a graph depicting an exemplary shear-dependent viscosity of a multi-component fluid material according to some embodiments.

FIG. 26A shows a schematic drawing of an exemplary steering element according to some embodiments.

FIG. 26B shows a schematic drawing a bent portion of a spraying device according to some embodiments.

FIG. 26C shows a schematic drawing of an exemplary steering element steering a bend portion of a spraying device according to some embodiments

FIG. 26D shows a schematic drawing of an exemplary steering element steering a bend portion of a spraying device according to some embodiments

FIG. 26E shows a schematic drawing of an exemplary steering element steering a bend portion of a spraying device according to some embodiments

FIG. 26F shows a schematic drawing of an exemplary steering element straightening a portion of a spraying device according to some embodiments

FIG. 27 shows an example of gas dispersant passageway outlet and nozzle outlet, according to some embodiments.

FIG. 28 shows a spray pattern with a spray angle and a spray diameter at a distance.

FIG. 29 shows a graph of a spray diameter at a spray distance.

FIG. 30 shows a stiffness comparison of a gel applied by different mechanisms.

FIG. 31A shows a dispersant outlet disposed proximal to a nozzle outlet.

FIG. 31B shows a dispersant outlet aligned with a nozzle outlet.

FIG. 31C shows a dispersant outlet distal to a nozzle outlet.

FIG. 32 illustrates a computer system that is programmed or otherwise configured to implement methods provided.

FIG. 33 provides an image of an example of a fluid jet from exiting a nozzle with a gas assisted delivery system, according to some embodiments.

FIG. 34 Shows a graph of the storage modulus of a fluid material dispensed from nozzles of differing embodiments, with a negative offset nozzle, a 0-offset nozzle, and a positive offset nozzle.

FIG. 35 shows a rear view of a fluid dispersion device according to some embodiments.

FIG. 36 shows a side view of a fluid dispersion device according to some embodiments.

FIG. 37 shows a front perspective view of a fluid dispersion device according to some embodiments.

FIG. 38 shows a rear perspective view of a fluid dispersion device according to some embodiments.

FIG. 39 shows front view of a fluid dispersion device according to some embodiments.

FIG. 40 shows a top view of a fluid dispersion device according to some embodiments.

FIG. 41 shows a cross sectional view of a nozzle with a 0 offset of some embodiments.

FIG. 42 shows the resulting aerosolization of water, glycerol, and ECM fluid through varying nozzles of some embodiments.

FIG. 43 shows a fluid dispersion of water through a MAD nozzle and graphs of the particle area and particle diameter distribution according to some embodiments.

FIG. 44 shows a fluid dispersion of water through a TYBR 0.6 nozzle and graphs of the particle area and particle diameter distribution according to some embodiments.

FIG. 45 shows a graph of the droplet diameter resulting from the fluid dispersion of water, glycerol, and ECM through the MAD nozzle, the TYBR 0.6 nozzle, and the TYBR 0.3 nozzle.

FIG. 46 shows a graph of the storage moduli of the ECM hydrogel when dispersed through a MAD nozzle, and TYBR 0.6 nozzle, and when injected.

FIG. 47 illustrates the effect of the ECM hydrogel when dispersed through the mixer with pressurized gas assist as compared to dispersion through a mixer without pressurized gas assist.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the term “generally” refers to a geometric relationship between two or more elements within tolerances of 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” is used to indicate that a value includes the inherent variation of error for the device, the method being employed to determine the value, or the variation that exists among the study subjects. Unless otherwise specified based upon the above values, the term “about” means ±5% of the listed value.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

### Introduction

Fibrous scar tissue can form on an individual organ, for example, on the small intestine, wherein the intestine can become strangulated by the band of scar tissue, which may lead to bowel obstruction. The scar may also form from one organ or tissue to another, causing for example, adhesion of the small intestine to the abdominal wall. Depending on the location of the surgical trauma, these adhesions can form on the intestines, female reproductive anatomy, on nerves in the abdominopelvic space, or in other major organ systems. In abdominal operations, adhesions can provoke severe problems such as chronic pain, infertility, and even bowel obstruction. Standard of care to avoid them may be with qualified surgical technique, reducing tissue and, especially, peritoneal trauma. In any of these cases, adhesions may prevent proper function of the organs or tissue to which they are bound, which may lead to devastating effects on the patient including small bowel obstruction, chronic visceral pain, and infertility. Surgical intervention and re-operation are the only treatment option available, and even with intervention the adhesion may reform after surgical removal.

Post-surgical adhesion formation may be reduced by minimizing peritoneal injury during surgery (i.e. careful surgical technique; gentle tissue handling, microsurgical principles), meticulous hemostasis, the excision of necrotic tissue, minimizing ischemia and desiccation, reducing cautery time, excising tissue rather than coagulating, frequent use of irrigation and aspiration, preventing the introduction of foreign bodies by using non-reactive suture materials, avoiding contamination with surgical glove powder, and the prevention of infection; and placing anti-adhesive barriers between damaged tissues. The use of anti-adhesion substances, or anti-adhesion barriers, may chemically, physically, biologically, or otherwise prevent postoperative adhesions from forming and preserve peritoneal, endometrial, joint, tendon, tissue, tissue function, or organ function during procedures with a risk of adhesions, or in a procedures at risk of complications resulting from adhesions.

Disclosed herein, are systems and methods for mixing and delivering a multi-component fluid. Each component of the multi-component fluid may be contained in a separate container. The method may comprise delivering a component of the multi-component fluid from a container to a multi-lumen tube (e.g., by using a plunging system), where the components of the multi-component fluid may be kept separate from one another. The components of the multi-component fluid may be subjected to mixing in a mixer. The multi-component fluid may be ejected from a nozzle positioned distal to a mixer (e.g., in the form of aerosols). A steering element may be used to steer the nozzle.

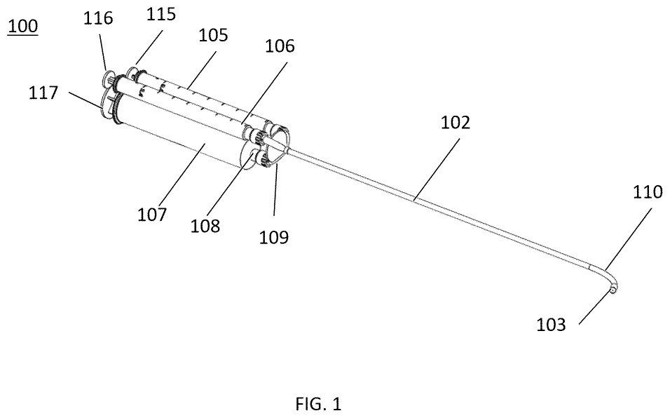

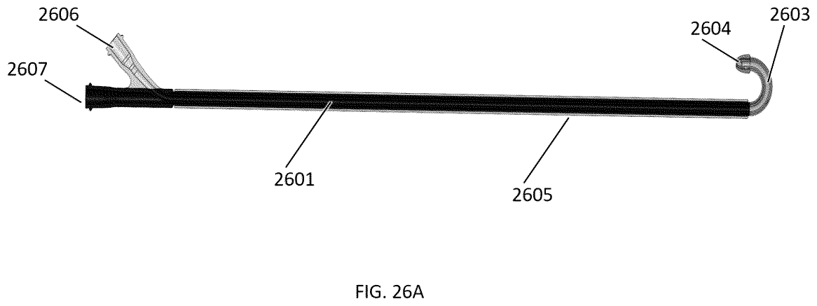

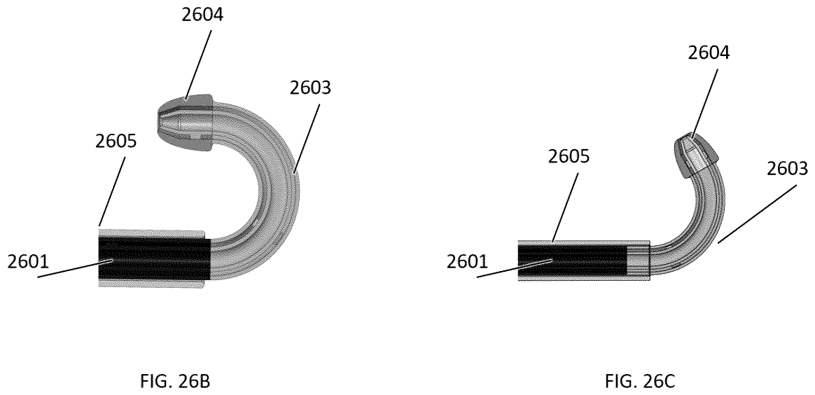

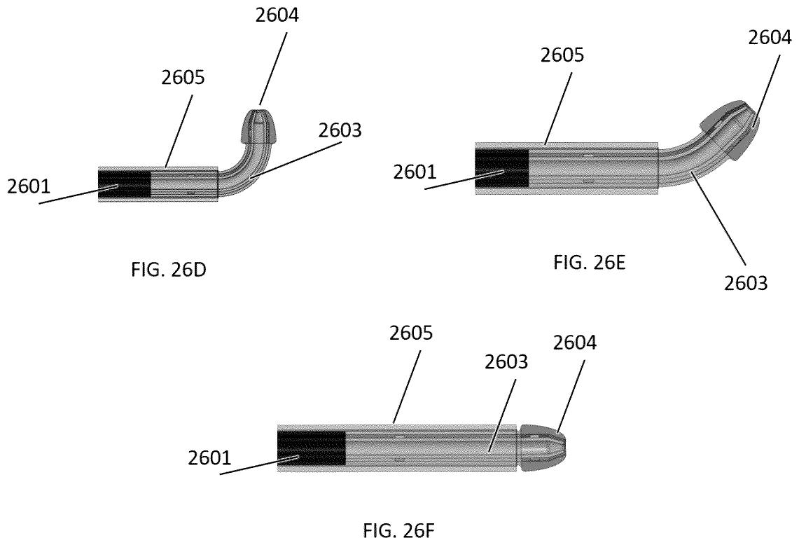

FIG. 1 shows an exemplary spray device 100 according to some embodiments, configured to mix and deliver a multi-component fluid. In some embodiments, the device comprises one or more fluid containers 105/106/107, a tube 102 in fluidic communication with the one or more fluid containers 105/106/107 and a portion 103 comprising a mixer and a nozzle. In some embodiments, each container 105/106/107 contains or may be configured to contain a fluid (e.g., a liquid component of the multi-component fluid, or a gas component). In some embodiments, the containers contain different amounts of fluids. In some embodiments, the container may be configured with different volumetric capacities. In some embodiments, a container 107 contains larger volume of fluid than a container 105 or 106. In some embodiments, a volumetric ratio between the amount of fluid contained in any two fluid containers (for example 105/106/107) may be about 5:1, 4:1, 3:1, 2:1, 1:1 or any ratio between any two ratios mentioned herein. In some embodiments, the volumetric ratio between the amount of fluid contained in any two containers (for example 105/106/107) may be at least about 5:1. In some embodiments, the volumetric capacity of a fluid container (for example 105/106/107) may be about 0.15 milliliter (mL) to about 24 mL. In some embodiments, the volumetric ratio between any two fluid chambers may be about 0.6 mL:0.15 ml to about 24 mL:6 mL. In some embodiments, the fluid contained in one or more containers may be a liquid. In some embodiments, the liquid contained in one container may be a pre-gel ECM mixture, and the liquid contained in another container may be a pH buffer. In some embodiments, the fluid contained in one or more containers may be a gel. In some embodiments, the fluid contained in one or more containers may be a dispersant (e.g., a gas). In some embodiments, a fluid container containing a dispersant comprising a volumetric capacity of about 1 mL to about 120 mL. In some embodiments, each fluid container has one end with an opening (for e.g., 108 of fluid container 107). In some embodiments, each opening may be in fluidic communication to a connector (e.g., 109), which may be in fluidic communication with tube 102. In some embodiments, each connector (e.g., 109) may be connected to the tube (see also reference characters 2606, 2607 in FIG. 26). In some embodiments, one or more containers (e.g., 105/106/107) may be removably coupled to the tube via a corresponding connector (e.g., 109). The one or more connectors may comprise a manifold. In some embodiments, the connector manifold couples one or more containers with the multi-lumen tube. In some embodiments, the connector 109 may be configured to connect a fluid source to the tube. The fluid source may be different from the one or more containers. In some embodiments, one or more fluid containers comprise a driver (e.g., a plunger 115, 116, and/or 117) configured to push a fluid out from a respective fluid container. In some embodiments, a driver may be a pressure. The pressure may be generated using a compressor. The pressure may be generated by storing high pressure fluid (e.g., pressurized gas) in a container. In some embodiments, a driver (e.g., a plunger) may be configured to move distally within a respective fluid container so as to be able to push the fluid out through the opening 108 of said respective fluid container at a specified rate. In some embodiments, two or more drivers (e.g., a plunger) may be configured to move at the same rate or at different rates within their respective fluid containers. In some embodiments, two or more drivers (e.g., a plunger 115, 116, and/or 117) of a fluid container may be operatively coupled to each other so as to ensure the drivers move within their respective fluid containers in unison. In some embodiments, the one or more drivers (e.g., a plunger) moving in unison enables the ratio of the volumetric rate of different components of the multi-component fluid entering into the tube from their respective containers to be constant or approximately constant (e.g., about 2:1 to about 8:1). In some embodiments, the one or more drivers (e.g., a plunger) moving in unison enables the ratio of the volumetric rate of different components of the multi-component fluid entering into the tube from their respective containers to be approximated about a fixed value (e.g., about 2:1 to about 8:1). In some embodiments, the one or more fluid containers may be configured to dispense a fluid automatically using, for example, an automatic actuation system. In some embodiments, the automatic actuation system comprises an automated dispenser. The automatic actuation system may comprise an electric motor or a pump. The automatic actuation system may enable the ratio of the volumetric rate of different components of the multi-component fluid entering into the tube from their respective containers to be approximated. In some embodiments, a volumetric ratio of rate of different components of the multi-component fluid entering into the tube from any two respective fluid containers (e.g., carrying a component of the multi-component fluid) may be about 2:1 to about 8:1. In some embodiments, the automated dispenser comprises a spring or a set of springs configured to depress the driver for one or more of the containers (e.g., plungers 115, 116, and/or 117) when triggered by the user (e.g., an operator). The spring may store mechanical energy in a compressed or stretched state. The spring may be stretched or compressed linearly or rotationally. The mechanical energy that may be stored in the spring may be released using a trigger operable by a user to move the drivers (e.g., a plunger). In some embodiments, the automated dispenser comprises an electrical, electronic, or electromechanical motor such as a stepper motor or other similarly controllable motor to drive the driver (e.g., a plunger). In some embodiments, a plurality of stepper motors or similarly controllable motors may be used to drive each of the drivers (e.g., a plunger) independently. In some embodiments, the driver (e.g., a plunger) may be driven by a mechanism other than a motor, such as, for example, air pressure, or one or more solenoids. In some embodiments, the solenoid may comprised of a wire coil about a housing comprising a movable plunger which may be drawn in when an electrical current is passed through the coil and draws the plunger from a distal end of the device to a proximal end of the device to dispense the fluid. In some embodiments, the solenoid may be disposed on a surface of the device. In some embodiments, the solenoid may be disposed on a surface of the device proximal to the fluid containers (e.g. syringes). In some embodiments, the solenoid may be disposed on a surface of the device surrounding the fluid container. In some embodiments, the solenoid may be disposed on a surface of the device adjacent to the fluid. In some embodiments, the solenoid may be disposed on device housing. In some embodiments, a human operator manually drives different components of the multi-component fluid from their respective containers using the driver (e.g., by pushing a piston, or operating a syringe). In some embodiments, the automated dispenser comprises a constant force spring or a set of constant force springs configured to depress the driver for one or more of the containers (e.g., plungers when triggered by the user or operator). In some embodiments, a delivery system for applying a fluid or multi-component fluid mixture in a surgical setting enables consistent delivery or application of a fluid. Such a delivery system may reduce or eliminate a user-induced variability so that application may be consistent from one user to another. In some embodiments, a driver (e.g., a piston, or a plunger) may be configured to slide within the interior of a housing in a fluid container. In some embodiments, a driver may be configured to roll or move in a spiral movement (e.g., screw) within the interior of a housing in a fluid container. In some embodiments, a driver comprises an elongated rod and a driving member (e.g., a piston or a disc, roller) attached to a forward end of the elongated rod (e.g., an end closer to an end of the tube 102 connected to the container). In some embodiments, the forward end of the driver forms a tip that cooperates with the piston. In some embodiments, the piston forms a tight seal with the interior wall of the fluid container where the piston may travel a path through a length of the container. In some embodiments, a driver may be displaced with a one-way or two-way engagement. The driver may be displaced distally or proximally relative to the opening of a fluid container. In some embodiments, each container may be attached to a manifold which may in turn attach to the tube 102 (e.g., a multi-lumen tube). In some embodiments, a fluid in a fluid container may be pressurized (e.g., pressurized and stored in the container). In some embodiments, a source of pressure such as a gas may be applied to a fluid in a fluid container to generate a pressure sufficient enough to deliver a fluid from the respective container to the distal end of the tube.

In some embodiments, the tube 102 comprises a multi-lumen tube (as described herein). In some embodiments, the tube 102 carries two or more components of the multi-component fluid to a mixer which may be in fluid communication with a nozzle. In some embodiments, the portion 103 shown in FIG. 1 comprises the mixer and the nozzle. The fluid (e.g., a mix of fluids, such as a multi-component fluid) may be aerosolized before, during or after delivery of the fluid out of the nozzle outlet. The distal portion of the tube 110 may be affixed as a separate portion of the multi-lumen tube. In some embodiments, the portion 110 may be configured to have spring-like properties. The portion 110 may be configured to be used in the steering process. In some embodiments, the system also comprises an automated dispenser mechanism which ejects the fluid from the storage containers automatically (e.g., without a need from a human operator).

In some embodiments, the multi-component fluid may be delivered to prevent postoperative adhesions. In some embodiments, the systems and method comprise mixing two fluids together within a spray device to form the multi-component fluid. In some embodiments, the multi-component fluid may be delivered through a nozzle as particles or droplets having a maximum dimension of 500 μm. In some cases, the particles have a dimension of about 10 μm to about 500 μm. In some embodiments, the particles have a maximum dimension of 300 μm. In some cases, the particles have a dimension of about 10 μm to about 20 μm, about 10 μm to about 30 μm, about 10 μm to about 50 μm, about 10 μm to about 100 μm, about 10 μm to about 150 μm, about 10 μm to about 200 μm, about 10 μm to about 300 μm, about 10 μm to about 400 μm, about 10 μm to about 500 μm, about 20 μm to about 30 μm, about 20 μm to about 50 μm, about 20 μm to about 100 μm about 20 μm to about 150 μm, about 20 μm to about 200 μm, about 20 μm to about 300 μm about 20 μm to about 400 μm, about 20 μm to about 500 μm, about 30 μm to about 50 μm, about 30 μm to about 100 μm, about 30 μm to about 150 μm, about 30 μm to about 200 μm about 30 μm to about 300 μm, about 30 μm to about 400 μm, about 30 μm to about 500 μm about 50 μm to about 100 μm, about 50 μm to about 150 μm, about 50 μm to about 200 μm about 50 μm to about 300 μm, about 50 μm to about 400 μm, about 50 μm to about 500 μm, about 100 μm to about 150 μm, about 100 μm to about 200 μm, about 100 μm to about 300 μm, about 100 μm to about 400 μm, about 100 μm to about 500 μm, about 150 μm to about 200 μm, about 150 μm to about 300 μm, about 150 μm to about 400 μm, about 150 μm to about 500 μm, about 200 μm to about 300 μm, about 200 μm to about 400 μm, about 200 μm to about 500 μm, about 300 μm to about 400 μm , about 300 μm to about 500 μm, or about 400 μm to about 500 μm. In some cases, the particles have a dimension of about 10 μm, bout 20 μm, about 30 μm, about 50 μm, about 100 μm, about 150 μm, about 200 μm, about 300 μm, about 400 μm, or about 500 μm. In some cases, the particles have a dimension of at least about 10 μm, about 20 μm, about 30 μm, about 50 μm, about 100 μm, about 150 μm, about 200 μm, about 300 μm, or about 400 μm. In some cases, the particles have a dimension of at most about 20 μm, about 30 μm, about 50 μm, about 100 μm, about 150 μm, about 200 μm, about 300 μm, about 400 μm, or about 500 μm. In some embodiments, the particles have a dimension of more than 500 μm or less than 10 μm. In some embodiments, a gas may be injected with the delivery of the multi-component fluid to aerosolize the fluid before and/or during delivery. In some embodiments, the gas may be stored in one of the fluid containers (e.g., similar to 105/106/107 in FIG. 1). In some embodiments, depression of a driver (e.g., plunger) for a container containing the gas creates pressure for delivering the gas through multi-lumen tube and dispersant passage of the mixer body (as described herein). In some embodiments, a gas (e.g., a dispersant) may be delivered from an external source (e.g., small gas cylinder, compressed air pump, medical air, wall air available in an operating room, or another gas source). In some embodiments, the gas (e.g., a dispersant) may be provided at least at 10 psi. Release and delivery of the gas (e.g., compressed air, nitrogen, CO2, etc.) to the nozzle of a spray device may be controlled and/or regulated by a controller. The controller may comprise an electronic or mechanical button and/or valve. The button may be placed on the spray device. The controller may comprise a pedal located near an operator's foot. In some embodiments, the controller may be a part of an automated dispenser, as described herein.

### Multi-Component Fluid

In some embodiments, the multi-component fluid comprises two or more components (e.g., two or more fluids) that are initially liquid, but are configured to turn into a gel or begin to gel upon or after mixing together. In some embodiments, the multi-component fluid may be as described in U.S. Patent App. No. PCT/US2021/020028 filed on Feb. 26, 2021 (Pub. No. WO2021174085A1), and U.S. Patent App. No. 62/983,520 filed on Feb. 28, 2020, the entire disclosures of which are hereby incorporated by reference.

In some embodiments, multi-component fluid may form or begin to form a gel after mixing and/or upon receiving heat. In some embodiments, multi-component fluid may form or begin to form a gel within a time frame after mixing. In some embodiments, multi-component fluid may form or begin to form a gel upon buffering to a neutral or a physiological pH (e.g. 7-7.4). In some embodiments, the multi-component fluid may form or begin to form a gel upon mixing and upon buffering to a physiological pH (e.g. 7-7.4).

In some embodiments, the delivery device mixes the components of the multi-component fluid and delivers the multi-component fluid to a target (e.g., a target tissue) prior to the multi-component fluid is fully gelled. The time frame for the gel to form may be from about 1 second (s) to about 5 minutes (m). In some cases, it may be considered that a gel has formed when it has reached about 50% of its fully gelled stiffness. In some embodiments, the multi-component fluid gels in about 1 s to about 300 s after mixing. In some embodiments, the multi-component fluid gels in about 1 s to about 2 s after mixing, about 1 s to about 3 s after mixing, about 1 s to about 5 s after mixing, about 1 s to about 10 s after mixing, about 1 s to about 20 s after mixing, about 1 s to about 30 s after mixing, about 1 s to about 40 s after mixing, about 1 s to about 50 s after mixing, about 1 s to about 100 s after mixing, about 1 s to about 200 s after mixing, about 1 s to about 300 s after mixing, about 2 s to about 3 s after mixing, about 2 s to about 5 s after mixing, about 2 s to about 10 s after mixing, about 2 s to about 20 s after mixing, about 2 s to about 30 s after mixing, about 2 s to about 40 s after mixing, about 2 s to about 50 s after mixing, about 2 s to about 100 s after mixing, about 2 s to about 200 s after mixing, about 2 s to about 300 s after mixing, about 3 s to about 5 s after mixing, about 3 s to about 10 s after mixing, about 3 s to about 20 s after mixing, about 3 s to about 30 s after mixing, about 3 s to about 40 s after mixing, about 3 s to about 50 s after mixing, about 3 s to about 100 s after mixing, about 3 s to about 200 s after mixing, about 3 s to about 300 s after mixing, about 5 s to about 10 s after mixing, about 5 s to about 20 s after mixing, about 5 s to about 30 s after mixing, about 5 s to about 40 s after mixing, about 5 s to about 50 s after mixing, about 5 s to about 100 s after mixing, about 5 s to about 200 s after mixing, about 5 s to about 300 s after mixing, about 10 s to about 20 s after mixing, about 10 s to about 30 s after mixing, about 10 s to about 40 s after mixing, about 10 s to about 50 s after mixing, about 10 s to about 100 s after mixing, about 10 s to about 200 s after mixing, about 10 s to about 300 s after mixing, about 20 s to about 30 s after mixing, about 20 s to about 40 s after mixing, about 20 s to about 50 s after mixing, about 20 s to about 100 s after mixing, about 20 s to about 200 s after mixing, about 20 s to about 300 s after mixing, about 30 s to about 40 s after mixing, about 30 s to about 50 s after mixing, about 30 s to about 100 s after mixing, about 30 s to about 200 s after mixing, about 30 s to about 300 s after mixing, about 40 s to about 50 s after mixing, about 40 s to about 100 s after mixing, about 40 s to about 200 s after mixing, about 40 s to about 300 s after mixing, about 50 s to about 100 s after mixing, about 50 s to about 200 s after mixing, about 50 s to about 300 s after mixing, about 100 s to about 200 s after mixing, about 100 s to about 300 s after mixing, or about 200 s to about 300 s. In some embodiments, the multi-component fluid gels in about 1 s, about 2 s, about 3 s, about 5 s, about 10 s, about 20 s, about 30 s, about 40 s, about 50 s, about 100 s, about 200 s, or about 300 s after mixing. In some embodiments, the multi-component fluid gels in about 300 s to about 1000 s after mixing. In some embodiments, the multi-component fluid gels in about 1000 s after mixing. In some embodiments, the gel may reach a maximum strength (e.g., storage modulus) in between 5 minutes (min) to 30 min after gel initiates to form. In some embodiments the multi-component fluid may be a shear thinning fluid, and experiences a reduction in viscosity under shear strain (e.g., mixing). In some embodiments the multi-component fluid comprises an acidic pregel which is buffered to a biological pH when it is mixed with a buffer solution and forms a gel with an increased storage modulus. In some embodiments the multi-component fluid comprises an acidic ECM pregel which gels into a crosslinked ECM hydrogel scaffold when mixed and buffered to a neutral or physiological pH (e.g., 7-7.4). In some cases, the storage modulus may be between 1500 to 3000 Pa. In some case the storage modulus may be about 2500 Pa.

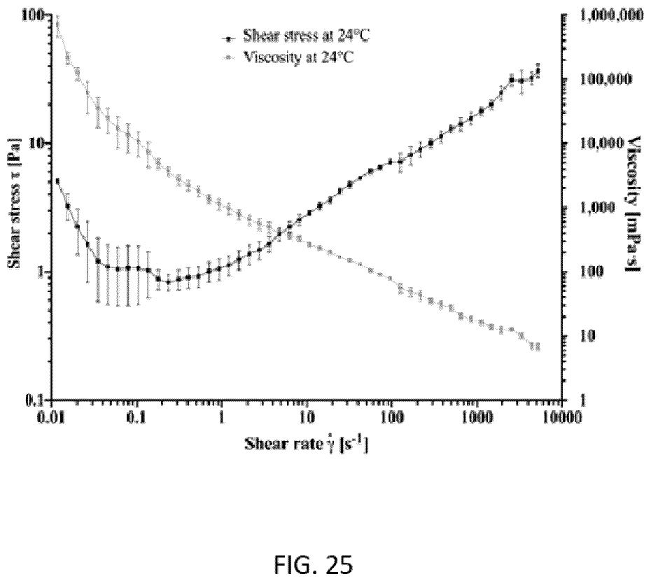

The multi-component fluid may comprise an adhesion barrier material. In some embodiments, the multi-component fluid may comprise a tissue-derived gel. In some embodiments, a first component comprising a tissue-derived stimuli-responsive gel with a viscosity ranging from about 5 centipoise (cP) to about 1,000,000 cP (1 cP=1 mPa*s) may be mixed with a second component comprising a buffer solution with a dynamic viscosity ranging from 0.5 cP to about 2.0 cP, to form a multi-component fluid. The multi-component fluid may form or begin to form a gel upon mixing. In some embodiments, the multi-component fluid (e.g., gel) has a viscosity similar to that of the tissue-derived stimuli-responsive gel. In some embodiments, a first component (e.g., first fluid) having a first viscosity range may be mixed with a second component (e.g., second fluid) having a second viscosity range, to form a multi-component fluid having a third viscosity range that encompasses the first and second viscosity ranges. In some embodiments, two or more fluids with viscosities similar to the gel and the buffer solution, described herein, may be mixed to form a multi-component fluid. In some embodiments, the multicomponent fluid has a viscosity of about 0.5 cP to about 1,000,000 cP. In some embodiments, generating a sheer stress in the multi-component fluid (e.g., by mixing) changes a viscosity of the multi-component fluid. FIG. 25 shows a graph depicting an exemplary shear-dependent viscosity of a multi-component fluid material. The data in FIG. 25 was generated using a rheometer. A set shear rate was applied and gradually increased. The resistance to motion (viscosity) was measured at each shear rate.

In some embodiments, the multi-component fluid comprises a natural polymeric material, polymeric material derived from a natural source, a synthetic polymeric material, or any combination thereof In some embodiments, natural polymeric materials comprise collagen, gelatin, fibrin, alginate, agar, cassava, maize, chitosan, gellan gum, corn-starch, chitin, cellulose, chia (Salvia hispanica) recombinant silk, decellularized tissue (plant or animal), hyaluronic acid, glycosaminoglycans, fibronectin, laminin, hemicellulose, glucomannan, textured vegetable protein, heparan sulfate, chondroitin sulfate, tempeh, keratan sulfate, or any combination thereof. In some embodiments, synthetic materials comprise hydroxyapatite, polyethylene terephthalate, acrylates, polyethylene glycol, polyglycolic acid, polycaprolactone, polylactic acid, their copolymers, or any combination thereof. In some embodiments, the multi-component fluid comprises a hydrogel, such as alginate. In some embodiments, the multi-component fluid comprises cellulose, cellulose derivatives, gelatin, acrylic resins, glass, silica gels, polyvinyl pyrrolidine (PVP), co-polymers of vinyl and acrylamide, polyacrylamides, latex gels, dextran, crosslinked dextrans (e.g., Sephadex™), rubber, silicon, plastics, nitrocellulose, natural sponges, metal, and agarose gel (Sepharose™). In some embodiments, the multi-component fluid comprises a biomaterial such as silk, poly(ethylene glycol), agarose, polylactic acid, poly (acryl acmide), diacrylate, poly (vinyl acid), poly(lactic co-glycolic acid), poly (methyl methacrylate), lipids, metals, cellulose, chitin, chitosan, collagen, gelatin, fibrin, alginate, agar, cassava, maize, gellan gum, corn-starch, chia (Salvia hispanica), decellularized tissue (plant or animal), hyaluronic acid, fibronectin, laminin, hemicellulose, glucomannan, textured vegetable protein, heparan sulfate, chondroitin sulfate, keratan sulfate, pectin, lignin, Matrigel, or any combination thereof. In some embodiments, the multi-component fluid comprises a synthetic fluid, synthetic gel, buffer solution, natural fluid, or a natural gel such as a tissue-derived gel. A tissue derived gel may be autologous or allogenic in origin. A tissue derived gel may be blended with a synthetic gel or synthetic fluid. In some embodiments, the multi-component fluid comprises an extracellular matrix (ECM) gel. In some embodiments, a tissue derived gel comprises an extracellular matrix pre-gel and a pH buffer. The buffer may comprise a base (e.g., NaOH), a salt (e.g., PBS), or a combination thereof, or other biologically acceptable pH buffered solutions. In some embodiments, an extracellular matrix gel comprises a tissue-derived stimuli-responsive gel. In some embodiments, the multi-component fluid comprises a smart material which may exhibit responsiveness to external stimuli including temperature, pH, ionic concentration, light, magnetic fields, electrical fields, chemicals, or enzymes.

### Tube

As described herein, in some embodiments, a spray device comprises tube 102. In some embodiments, the tube may be in fluidic communication with one or more containers, as described herein. In some embodiments, the tube may be configured to deliver one or more fluid components from the one or more containers to an outlet of the spray device (e.g., a tube opening, mixer, a nozzle). In some embodiments, the tube comprises a multi-lumen tube. A multi-lumen tube may comprise at least 2, 3, 4, 5, 6, 7, 8, 9, 10, or more lumens. In some embodiments, the multi-lumen tube may comprise at most about 10, 9, 8, 7, 6, 5, 4, 3, or 2 lumens. In some embodiments, the one or more lumens of the multi-lumen tube may be formed through extrusion. In some embodiments, the extruded lumens have increased elastic moduli, a highly uniform cross sectional area, increased flexibility, increased mechanical properties, are smooth along the lateral surface of the lumen, and have a low coefficient of friction. In some embodiments, the one or more lumens comprise separate tubes within the multi-lumen tube. A multi-lumen tube may carry one or more fluid components. One or more lumen of a multi-lumen tube may carry a different fluid component of the multi-component fluid. In some embodiments, each lumen of the multi-lumen may be in fluidic communication with a respective container (e.g., 105/106/107). The different fluid components in a multi-lumen tube may not mix or contact one another. The multi-lumen tube may be used to keep the one or more fluid components separate. The separate fluid components may travel through the multi-lumen tube at substantially the same rate. In some embodiments, one or more fluid components may travel through the multi-lumen tube at a different rate compared to other fluid components. For example, a dispersant (e.g., a gas) delivered via the dispersant passageway to the outlet may travel at a different rate or with a time delay compared to two or more fluid components that are mixed in the mixer. In some embodiments, the separate fluid components travel through the multi-lumen tube at the same rate. In some embodiments, two or more of the lumens of a multi-lumen tube may have a similar cross-section or they may have a different cross-section. The cross-sectional area of two or more lumens may be a ratio of approximately 1:1, 2:1, 3:1, 4:1, 5:1, 6:1, 7:1, 8:1, 9:1, 10:1, or greater than 10:1. In some embodiments, each lumen of the multi-lumen may be configured to span a portion of a tube length. In some embodiments, each lumen spans at least about 60% to about 100% of the tube length. In some embodiments, each lumen spans from a proximal end of the tube to a location distal to the distal end of the tube. In some embodiments, one or more lumens of the multi-lumen tube spans a different length from the other lumens.

In some embodiments, the multi-lumen tube has a length of about 1 centimeter (cm) to about 100 cm. In some embodiments, a multi-lumen tube may have a length of about 1 cm to about 5 cm, about 1 cm to about 10 cm, about 1 cm to about 20 cm, about 1 cm to about 30 cm, about 1 cm to about 40 cm, about 1 cm to about 50 cm, about 1 cm to about 60 cm, about 1 cm to about 70 cm, about 1 cm to about 80 cm, about 1 cm to about 90 cm, about 1 cm to about 100 cm, about 5 cm to about 10 cm, about 5 cm to about 20 cm, about 5 cm to about 30 cm, about 5 cm to about 40 cm, about 5 cm to about 50 cm, about 5 cm to about 60 cm, about 5 cm to about 70 cm, about 5 cm to about 80 cm, about 5 cm to about 90 cm, about 5 cm to about 100 cm, about 10 cm to about 20 cm, about 10 cm to about 30 cm, about 10 cm to about 40 cm, about 10 cm to about 50 cm, about 10 cm to about 60 cm, about 10 cm to about 70 cm, about 10 cm to about 80 cm, about 10 cm to about 90 cm, about 10 cm to about 100 cm, about 20 cm to about 30 cm, about 20 cm to about 40 cm, about 20 cm to about 50 cm, about 20 cm to about 60 cm, about 20 cm to about 70 cm, about 20 cm to about 80 cm, about 20 cm to about 90 cm, about 20 cm to about 100 cm, about 30 cm to about 40 cm, about 30 cm to about 50 cm, about 30 cm to about 60 cm, about 30 cm to about 70 cm, about 30 cm to about 80 cm, about 30 cm to about 90 cm, about 30 cm to about 100 cm, about 40 cm to about 50 cm, about 40 cm to about 60 cm, about 40 cm to about 70 cm, about 40 cm to about 80 cm, about 40 cm to about 90 cm, about 40 cm to about 100 cm, about 50 cm to about 60 cm, about 50 cm to about 70 cm, about 50 cm to about 80 cm, about 50 cm to about 90 cm, about 50 cm to about 100 cm, about 60 cm to about 70 cm, about 60 cm to about 80 cm, about 60 cm to about 90 cm, about 60 cm to about 100 cm, about 70 cm to about 80 cm, about 70 cm to about 90 cm, about 70 cm to about 100 cm, about 80 cm to about 90 cm, about 80 cm to about 100 cm, or about 90 cm to about 100 cm. In some embodiments, a multi-lumen tube may have a length of about 1 cm, about 5 cm, about 10 cm, about 20 cm, about 30 cm, about 40 cm, about 50 cm, about 60 cm, about 70 cm, about 80 cm, about 90 cm, or about 100 cm. the multi-lumen tube has a length of about 30 centimeter (cm) to about 40 cm. In some embodiments, a multi-lumen tube may have a length of at least about 1 cm, about 5 cm, about 10 cm, about 20 cm, about 30 cm, about 40 cm, about 50 cm, about 60 cm, about 70 cm, about 80 cm, or about 90 cm. In some embodiments, a multi-lumen tube may have a length of at most about 100 cm, about 90 cm, about 80 cm, about 70 cm, about 60 cm, about 50 cm, about 40 cm, about 30 cm, about 20 cm, about 10 cm, about 5 cm, about 1 cm, or less. In some embodiments, the device

In some embodiments, the outer diameter of the multi-lumen tube may be varied to accommodate various surgical port sizes. The outer diameter of the multi-lumen tube may be about 1 millimeter (mm) to about 50 mm. The outer diameter of the tube may be about: 1 mm, 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, or more than 50 mm. In some embodiments, the outer diameter of the tube may be a diameter between any of the two diameters mentioned herein or a diameter less than 1 mm. In some embodiments, the outer diameter of the multi-lumen tub may be between 5 mm to about 12 mm, about 5 mm to about 8 mm, about 8 mm to about 12 mm. In some embodiments, the outer diameter of the multi-lumen tub may be 5 mm, 8 mm, or 12 mm.

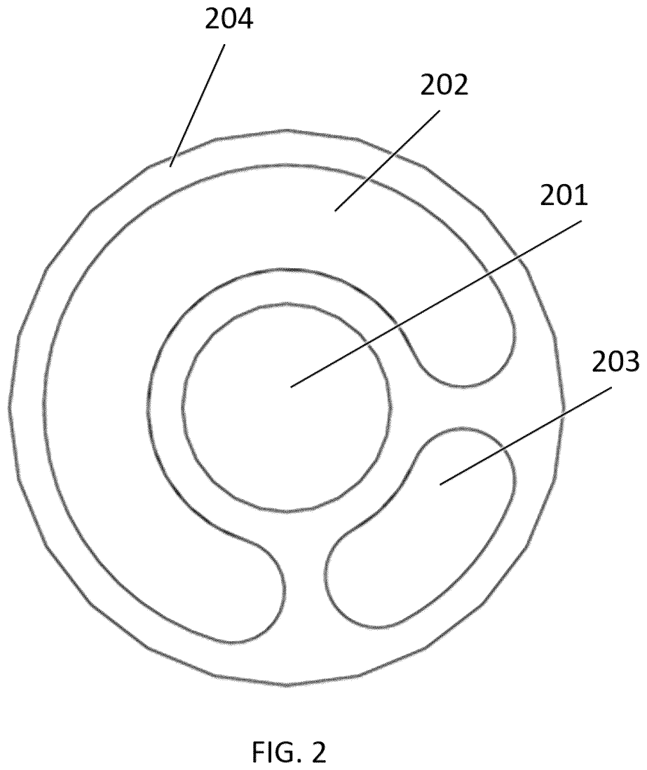

FIG. 2 shows an example of a cross-section of a multi-lumen tube comprising three lumens. The lumens 201, 202, or 203 may have different cross-sectional areas. In some embodiments, two or more of the lumens 201, 202, or 203 may have a similar cross-sectional area. In some embodiments, a dispersant lumen 201 may carry a dispersant (e.g., a gas). As described herein, in some embodiments, the dispersant lumen receives a dispersant from an external gas source (e.g., a compressor) instead of a container coupled to the tube. In some embodiments, the two outer lumens 202 and 203 may carry other fluid components. In some embodiments, the lumens have different cross-sectional areas. In some embodiments, the two outer lumens comprise a cross-sectional area ratio of 4:1 for carrying respective types of fluid. In some embodiments, the types of fluid include an extracellular matrix pre-gel and a pH buffer. The extracellular matrix pre-gel may pass through the larger lumen. For example, a cross-sectional area ratio of 202 to 203 may be about 4:1 for carrying two fluid components (e.g., an extracellular matrix pre-gel and a pH buffer) at a ratio of 4:1, as described hereinbefore. The ratio of the cross section of 202 to 203 may be 3:1, 5:1, 2:1, 1:1 or any ratio between any two ratios mentioned herein. The multi-lumen tube may also comprise a wall 204. The wall 204 may separate the lumens from one another. The wall 204 may comprise a soft or a hard material. The wall 204 may comprise a bendable material. In some embodiments, bending the wall 204 may not change a cross section area of the lumens. In some embodiments, lumens may stay in the same relative position within the tube when bending without breaking or cracking and may maintain the cross sectional area of the lumens. In some embodiments, the walls of the tube may expand and bend as the tube may be bent. In some embodiments, the tube may be composed of a flexible, high strength polymer material.

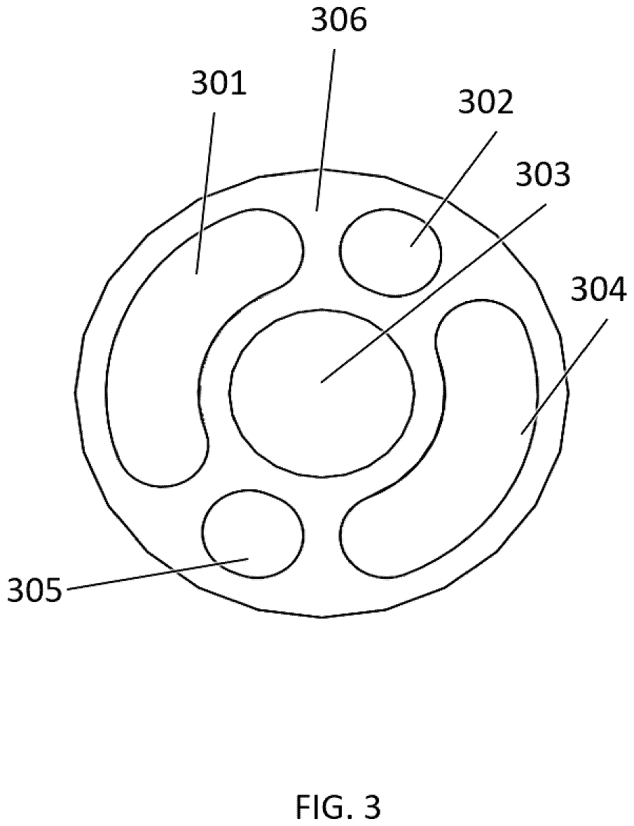

FIG. 3 shows an example of a cross-section of a multi-lumen tube comprising five lumens. The lumens 301, 302, 303, 304, or 305 may have different cross-sectional areas. The cross-sectional area between any two of the mentioned lumens may have a ratio of 1:1, 2:1, 3:1, 4:1, 5:1 or any ratio between any two ratios mentioned herein. In some embodiments, two or more of the lumens 301, 302, 303, 304, or 305 may have a similar cross-sectional area. For example, a lumen 301 may have a similar cross-section as the lumen 304. 302 and 305 may also have a similar cross-sectional area. In some embodiments, a dispersant lumen 303 may carry a dispersant (e.g., a gas). A wall 306 may separate the lumens of the multi-lumen tube. The wall 306 may comprise a soft or hard material.

In some embodiments, the tube comprises a tube opening located at a distal end. In some embodiments, the tube opening may be configured as a nozzle as described herein. In some embodiments, the tube opening may be coupled to a nozzle as described herein. In some embodiments, the tube opening may be coupled to a mixer. In some embodiments, as described herein, a mixer may be disposed within the tube and located distal to the tube opening. In some embodiments, the mixer may be disposed within a distal end of the tube and located proximal to the tube opening or a nozzle.

In some embodiments, the tube may be coupled to a nozzle, mixer, or other components using an adhesive. An adhesive may comprise a structural adhesive, pressure sensitive adhesive, thermosetting adhesive, epoxy, polyurethane, polyimides, paste, liquid, film, pellet, tape, hot melt adhesive, contact adhesive, reactive hot melt adhesive, cyanoacrylate, urethanes, acrylics, glue, resin, anaerobic, Krazy Glue®, cyanoacrylate glue, hot glue, polyvinyl acetate, silicones, phenolics, instant glues, plastisols or another chemical joint. In some embodiments, the multi-lumen tube may be coupled to a mixer, nozzle, other components using barbed tubing fittings or other mechanical joint. In some embodiments, the multi-lumen tube may be coupled to a mixer, nozzle, or other components using a weld. In some embodiments, the weld may be performed using ultrasonic welding, thermal welding, or other method of fusing. In some embodiments, the multi-lumen tube may be affixed to adjacent components with adhesive, barbed tubing fittings, a weld, or any combination thereof.

### Mixer

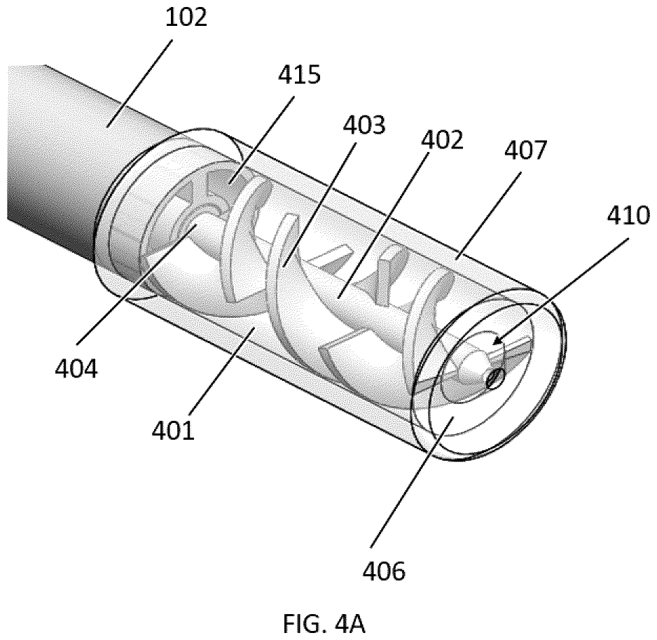

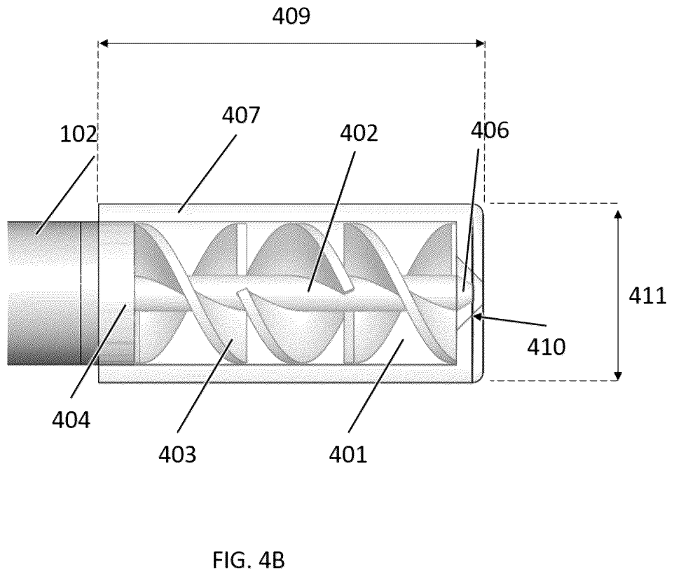

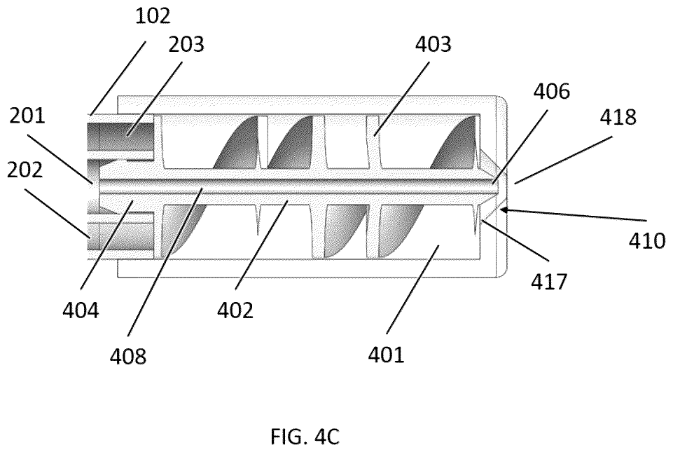

In some embodiments, the spray device comprises a mixer. The mixer may be configured to mix two or more fluid components of the multi-component fluid. In some embodiments, the mixer may be configured to mix an extra cellular matrix pre-gel material (ECM) and pH buffer. The mixed fluid may form a homogeneous fluid after being mixed by the mixer. In some embodiments, the mixer may be a static mixer. In some embodiments, the mixer may be disposed within the tube. In some embodiments, the mixer may be disposed distal to the tube end or a distal portion of the tube thereof (e.g., portion 110). In some embodiments, the mixer comprises a chamber and a mixer body. The chamber may be disposed within a housing. In some embodiments, the mixer body may be disposed within the chamber. In some embodiments, the mixer body may be disposed within the housing. In some embodiments, the housing may be coupled to the tube 102. In some embodiments, the nozzle may be located within the chamber and distal to the mixer body. In some embodiments, a portion of the nozzle may be located within the chamber and distal to the mixer body. In some embodiments, a body of the nozzle may be disposed in the housing and distal to the mixer body. In some embodiments, the mixer may be configured to receive two or more fluid components from the multi-lumen tube and mix the two or more fluid components. In some embodiments, one or more lumens terminate at or proximal to the mixer. In some embodiments, the fluid components are driven towards the nozzle by the pressure provided from a driving force attached to the fluid containers (e.g., the plunger in the plunging system), a force generated by the movement of the mixer, or a combination of both. The mixer may substantially completely mix the two or more fluid components (e.g., a substantially homogenized mix) prior to the multi-component fluid being delivered from the spray device. In some embodiments, a mixer comprises a mixer body. In some embodiments, the mixer body comprises a central shaft. The mixer body may comprise a mixing element. In some embodiments, the one or more mixing elements may be attached to the central shaft. The one or more mixing elements may comprise a baffle, a blade, a fin, or a channel. In some embodiments, the mixer receives one or more fluids from the multi-lumen tube. In some embodiments, the central shaft comprises a cavity therein. The cavity disposed within the central shaft may be a dispersant passageway. In some embodiments, the cavity comprises an annular cavity. In some embodiments, the cavity may be configured to receive a fluid (e.g., a gas) such as a dispersant from the multi-lumen tube. The cavity disposed within the central shaft may be a dispersant passageway. In some embodiments, the dispersant fluid may travel from a dispersant fluid source through a lumen of a multi-lumen tube, through the cavity (e.g., dispersant passageway), thereby bypassing the mixing elements of the mixer, and delivered from the spray device through the nozzle. The dispersant fluid source may be a dispersant fluid container. The dispersant fluid source may comprise a compressor providing the dispersant to the multi-lumen tube (e.g., a compressed gas). The dispersant passageway may keep the dispersant separated from the fluid components of the multi-component fluid as they are being mixed. The dispersant passageway may deliver and release the dispersant at a position proximal to a nozzle outlet (shown as 418 in FIG. 4C), so as to be delivered with the mixed multi-component fluid.

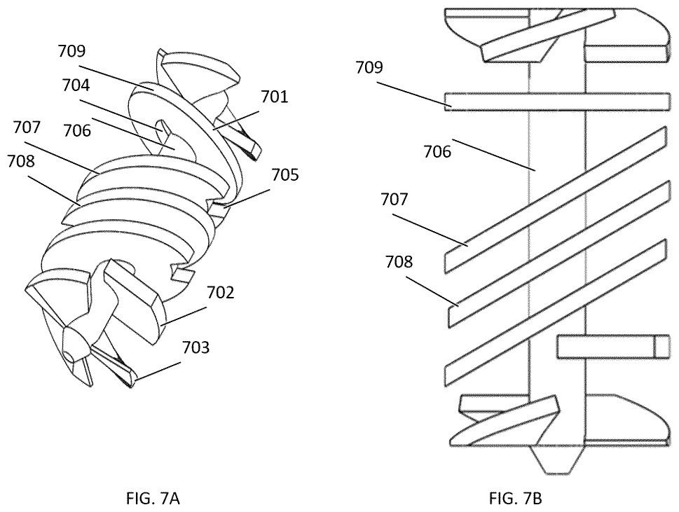

FIG. 4A-4C show an exemplary depiction of a mixer. FIG. 4A, shows the mixer comprising a chamber 401 disposed within a housing 407, and a mixer body disposed within the chamber. The mixer may be coupled to the tube 102. The mixer body may comprise a central shaft 402, and a plurality of mixing elements 403 (e.g., baffles, blades, discs as described herein, and/or other structures configured to mix two or more fluids) extending therefrom. The mixer body may comprise a plurality of mixing elements such as baffles (e.g., ref. char. 702 from FIG. 7A) and/or blades 403 spaced apart along the central shaft. In some embodiments, a blade 403 wraps around a central shaft. In some embodiments, a baffle crosses a central shaft. The curvature of the baffles and/or blades may be clockwise or counterclockwise. The baffles and/or blades may revolve around the central shaft where the angle of revolution may be between about 5° to about 360° (e.g., a full rotation or revolution). A blade 403 may revolve around the central shaft between 0.1 to 30 full rotations. In some embodiments, a blade 403 revolves around the central shaft between about 0.1 to about 0.9 full rotation, about 1.1 to about 2.9 full rotation, or about 0.5 to about 3.5 full rotation. In some embodiments, a blade 403 revolves around the central shaft less than 0.1 full rotation, or more than 30 full rotations. In some embodiments, the housing 407 may be coupled to the tube 102. In some embodiments, a proximal end 404 of the central shaft 402 may be coupled to the dispersant lumen of the multi-lumen tube. In some embodiments, the mixer may be coupled to the tube via the mixer chamber housing 407 using a weld. In some embodiments, the weld may be performed using ultrasonic welding, thermal welding, or other method of fusing. In some embodiments, the mixer housing may be affixed to the multi-lumen tube with adhesive, barbed tubing fittings, a weld, or any combination thereof. In some embodiments, a proximal end of the mixer may be attached to a tube and a distal end of the mixer may be attached to a or a nozzle. The mixer may be configured to meet a nozzle 410 at a distal end of the mixer 406. FIG. 4B shows a side view of the mixer comprising a housing 407, the chamber 401, the central shaft 402, a plurality of blades 403, the mixer end 404 coupled to the multi-lumen tube of the tube 102, the mixer body distal end 406 proximal to a nozzle 410. In some embodiments, the housing 407 may be circular, elliptical, rectangular, spherical, cylindrical, or a combination thereof. In some embodiments, the mixer may be an inline mixer disposed within the tube.

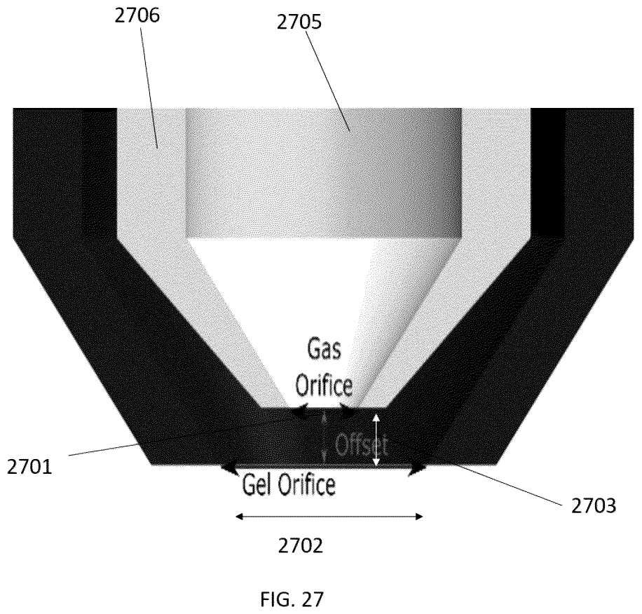

The housing 407 may have a length 409. FIG. 4C shows a cross-sectional view of the mixer from FIGS. 4A-B. The central shaft 402 may comprise a cavity 408 therein, as described herein. In some embodiments, the cavity 408 comprises an annular cavity. In some embodiments, the cavity 408 may be in fluidic communication with a lumen of the multi-lumen tube. In some embodiments, a fluid (e.g., a dispersant) being transported in a dispersant lumen 201 (e.g., 201 or 303) may enter the annular cavity 408 at the end 404. The cavity 408 may be a dispersant passageway, as described herein. The cavity 408 may be open at a distal end 406 of the dispersant passageway. In some embodiments, the distal end 406 of the dispersant passageway defines a dispersant outlet 413. In some embodiments, the dispersant outlet comprises an orifice. The dispersant fluid being transported in the dispersant lumen or cavity 408 may be released through the dispersant outlet 413 from the distal end 406 of the dispersant passageway into the nozzle 410. In some embodiments, the dispersant outlet 413 has a diameter smaller than the nozzle outlet diameter 410. FIG. 27 provides an exemplary depiction of the relation of the dispersant outlet diameter (reference character 2701) and the nozzle outlet diameter (reference character 2702).

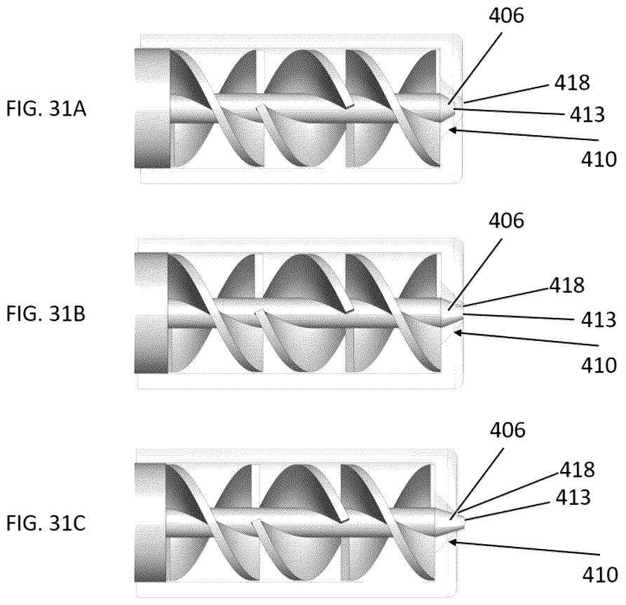

In some embodiments, the tapered portion of distal end 406 (as shown in FIGS. 4B-C) may be a part of the mixer body (for e.g., a part of the mixer body that extends distally in a tapered manner). In some embodiments, the tapered portion of distal end 406 may be a dispersant nozzle coupled to a distal end of the mixer body. In some embodiments, the distal end 406 (e.g., as part of the mixer body or as a dispersant nozzle coupled to the mixer body) may extend to a location within the nozzle 410 wherein the dispersant outlet may be located proximal to the nozzle outlet (for e.g., see FIGS. 4B-C, and FIG. 31A). In this embodiment (e.g., FIG. 31A), the dispersant may be released proximal to the nozzle outlet 418. In some embodiments, the distal end 406 (e.g., as part of the mixer body or as a dispersant nozzle coupled to the mixer body) may extend to a location that may be co-planar with the nozzle outlet (e.g., see FIG. 31B), wherein the dispersant outlet may be aligned with the nozzle outlet. In this embodiment, the dispersant may be released at the same location of the nozzle outlet 418. In some embodiments, the distal end 406 (e.g., as part of the mixer body or as a dispersant nozzle coupled to the mixer body) may extend to a location that may be located distal to the nozzle outlet (for e.g., see FIG. 31C). In this embodiment (e.g., FIG. 31C), the dispersant may be released distal to the nozzle outlet 418, and thus may interact with the multi-component fluid once it has been delivered through nozzle outlet. The chamber 401 may be in fluid communication with two or more lumens of the multi-lumen tube. Two or more fluid components may be delivered from two or more lumens (e.g., 202 or 203) from the multi-lumen tube of the tube 102 into the chamber 401 at the end 404. In some embodiments, the blades 403 as shown in FIG. 4C are configured to mix the two or more fluids received from the two or more lumens, such that a mixture of the fluids (e.g., multi-component fluid) may be delivered to the nozzle 410. The fluid mixture may be delivered to the nozzle 410 via the nozzle inlet 417.

The nozzle inlet 417 may be aligned with the dispersant passageway 408. The nozzle inlet 417 and the dispersant passageway 408 may be disposed coaxially with respect to one another. The nozzle inlet 417 may be aligned with the dispersant passageway 408 in a way to form a gap for the mixed fluid to be delivered into the nozzle 410. In some cases, a portion 406 of the dispersant passageway may be disposed within the nozzle 410 (as shown in FIG. 4C). In some cases, a portion 406 of the dispersant passageway may be disposed proximal to the nozzle inlet 417 with a distance of at most about 2 millimeters (mm). The distance between the portion 406 of the dispersant passageway and the nozzle inlet 417 may be about 1 mm to about 3 mm, about 0.5 mm to about 2 mm, about 1 mm to about 2 mm, about 1.5 mm to about 1.8 mm, or about 0.1 mm to about 0.5 mm.

A housing of a mixer may have a length, for example, a length 409 as shown in FIG. 4B. The length of a mixer housing may be about 0.25 cm to about 6 cm. In some embodiments, length of a mixer housing may be about 0.5 cm to about 1 cm, about 0.5 cm to about 1.5 cm, about 0.5 cm to about 2 cm, about 0.5 cm to about 2.5 cm, about 0.5 cm to about 3 cm, about 0.5 cm to about 3.5 cm, about 0.5 cm to about 4 cm, about 0.5 cm to about 4.5 cm, about 0.5 cm to about 5 cm, about 0.5 cm to about 5.5 cm, about 0.5 cm to about 6 cm, about 1 cm to about 1.5 cm, about 1 cm to about 2 cm, about 1 cm to about 2.5 cm, about 1 cm to about 3 cm, about 1 cm to about 3.5 cm, about 1 cm to about 4 cm, about 1 cm to about 4.5 cm, about 1 cm to about 5 cm, about 1 cm to about 5.5 cm, about 1 cm to about 6 cm, about 1.5 cm to about 2 cm, about 1.5 cm to about 2.5 cm, about 1.5 cm to about 3 cm, about 1.5 cm to about 3.5 cm, about 1.5 cm to about 4 cm, about 1.5 cm to about 4.5 cm, about 1.5 cm to about 5 cm, about 1.5 cm to about 5.5 cm, about 1.5 cm to about 6 cm, about 2 cm to about 2.5 cm, about 2 cm to about 3 cm, about 2 cm to about 3.5 cm, about 2 cm to about 4 cm, about 2 cm to about 4.5 cm, about 2 cm to about 5 cm, about 2 cm to about 5.5 cm, about 2 cm to about 6 cm, about 2.5 cm to about 3 cm, about 2.5 cm to about 3.5 cm, about 2.5 cm to about 4 cm, about 2.5 cm to about 4.5 cm, about 2.5 cm to about 5 cm, about 2.5 cm to about 5.5 cm, about 2.5 cm to about 6 cm, about 3 cm to about 3.5 cm, about 3 cm to about 4 cm, about 3 cm to about 4.5 cm, about 3 cm to about 5 cm, about 3 cm to about 5.5 cm, about 3 cm to about 6 cm, about 3.5 cm to about 4 cm, about 3.5 cm to about 4.5 cm, about 3.5 cm to about 5 cm, about 3.5 cm to about 5.5 cm, about 3.5 cm to about 6 cm, about 4 cm to about 4.5 cm, about 4 cm to about 5 cm, about 4 cm to about 5.5 cm, about 4 cm to about 6 cm, about 4.5 cm to about 5 cm, about 4.5 cm to about 5.5 cm, about 4.5 cm to about 6 cm, about 5 cm to about 5.5 cm, about 5 cm to about 6 cm, or about 5.5 cm to about 6 cm. In some embodiments, length of a mixer housing may be about 0.5 cm, about 1 cm, about 1.5 cm, about 2 cm, about 2.5 cm, about 3 cm, about 3.5 cm, about 4 cm, about 4.5 cm, about 5 cm, about 5.5 cm, or about 6 cm. In some embodiments, length of a mixer housing may be at least about 0.5 cm, about 1 cm, about 1.5 cm, about 2 cm, about 2.5 cm, about 3 cm, about 3.5 cm, about 4 cm, about 4.5 cm, about 5 cm, about 5.5 cm, about 6 cm, or less. In some embodiments, length of a mixer housing may be at most about 6 cm, or about 5.5 cm, or about 5 cm, or about 4.5 cm, or about 4 cm, or about 3.5 cm, or about 3 cm, or about 2.5 cm, or about 2 cm, or about 1.5 cm, or about 1 cm, or about 0.5 cm, or less.

In some embodiments, a housing of a mixer, for example, the housing 407 as shown in FIG. 4B, may be circular, elliptical, rectangular, spherical, cylindrical, or a combination thereof in shape. The housing of a mixer housing may have a width or diameter 411. In some embodiments, the width or diameter may be about 2 mm to about 50 mm. In some embodiments, the width or diameter may be about 2 mm to about 4 mm, about 2 mm to about 6 mm, about 2 mm to about 8 mm, about 2 mm to about 10 mm, about 2 mm to about 12 mm, about 2 mm to about 15 mm, about 2 mm to about 20 mm, about 2 mm to about 30 mm, about 2 mm to about 50 mm, about 4 mm to about 6 mm, about 4 mm to about 8 mm, about 4 mm to about 10 mm, about 4 mm to about 12 mm, about 4 mm to about 15 mm, about 4 mm to about 20 mm, about 4 mm to about 30 mm, about 4 mm to about 50 mm, about 6 mm to about 8 mm, about 6 mm to about 10 mm, about 6 mm to about 12 mm, about 6 mm to about 15 mm, about 6 mm to about 20 mm, about 6 mm to about 30 mm, about 6 mm to about 50 mm, about 8 mm to about 10 mm, about 8 mm to about 12 mm, about 8 mm to about 15 mm, about 8 mm to about 20 mm, about 8 mm to about 30 mm, about 8 mm to about 50 mm, about 10 mm to about 12 mm, about 10 mm to about 15 mm, about 10 mm to about 20 mm, about 10 mm to about 30 mm, about 10 mm to about 50 mm, about 12 mm to about 15 mm, about 12 mm to about 20 mm, about 12 mm to about 30 mm, about 12 mm to about 50 mm, about 15 mm to about 20 mm, about 15 mm to about 30 mm, about 15 mm to about 50 mm, about 20 mm to about 30 mm, about 20 mm to about 50 mm, or about 30 mm to about 50 mm. In some embodiments, the width or diameter may be about 2 mm, about 4 mm, about 6 mm, about 8 mm, about 10 mm, about 12 mm, about 15 mm, about 20 mm, about 30 mm, or about 50 mm. In some embodiments, the width or diameter may be at least about 2 mm, about 4 mm, about 6 mm, about 8 mm, about 10 mm, about 12 mm, about 15 mm, about 20 mm, or about 30 mm. In some embodiments, the width or diameter may be at most about 50 mm, about 30 mm, about 20 mm, about 15 mm, about 12 mm, about 10 mm, about 8 mm, about 6 mm, about 4 mm, about 2 mm, or less.

In some embodiments, the mixer comprises a static mixer. The mixing elements in a mixer may have a variety of forms or shapes. In some embodiments, a mixing element may be configured as a circle, ellipse, oval, square, rectangle, rhombus, kite, triangle, pentagon, hexagon, heptagon, octagon, nonagon, decagon, star, heart, crescent, cross, pie, trapezoid, parallelogram or any combination thereof. A mixing element may comprise one or a series of blades, slots, plates, baffles, or channel. The mixer and the mixing elements may not move radially and/or axially. A mixture of fluids may be formed as two or more fluid components are delivered to the mixer and flow across the mixing elements. The mixing elements in a mixer may be configured to form an angle with a central shaft of the mixer. In some embodiments, a mixing element and a central shaft may form an angle therebetween of about 1°, 2°, 3°, 4°, 5°, 6°, 7°, 8°, 9°, 10°, 15°, 20°, 25°, 30°, 35°, 40°, 45°, 50°, 60°, 70°, 80°, to about 90°. The angle may be an angle between any two angles mentioned herein. The angle may be less than 1°, or more than 90°.

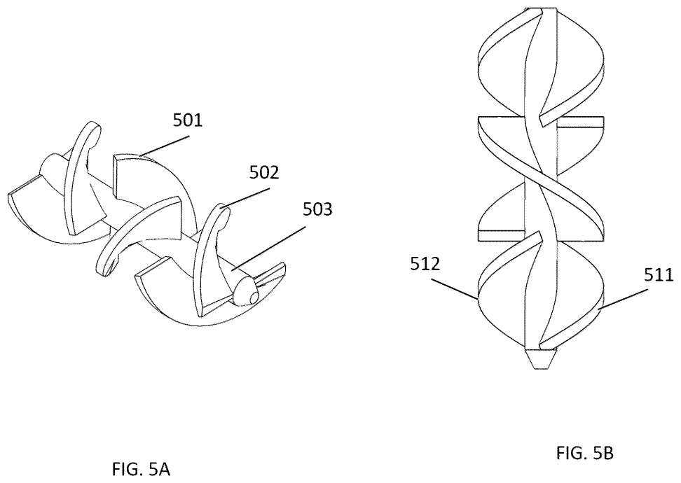

FIG. 5A depicts a perspective view of an example of a mixer body of a static mixer according to some embodiments. The mixer body may comprise a plurality of blades 501 or 502. The blades in a mixer body may be coupled to (e.g., wrapped around) a central shaft 503. In some embodiments, the plurality of blades 501 and 502 are spaced apart from one another. FIG. 5B depicts a side view of an example of a mixer body comprising baffles. In some embodiments, the baffles 512 and 511 are overlapping. In some embodiments, the blade may be configured to span an arc of about 1° to about 360°. In some embodiments, a blade mixing element may revolve around the central shaft 503 in a continuous manner to generate a spiral blade around the central shaft. In some embodiments, the mixer body may comprise two or more sections, where each section comprises a blade, or a baffle. In some embodiments, there may be a space or gap between two sections. The sections may be spaced such that they overlap. For example, a space between two sections may be less than a length of a blade. In some embodiments, two sections may not overlap. In some embodiment, a section may have a length of about 1 mm to about 10 mm.

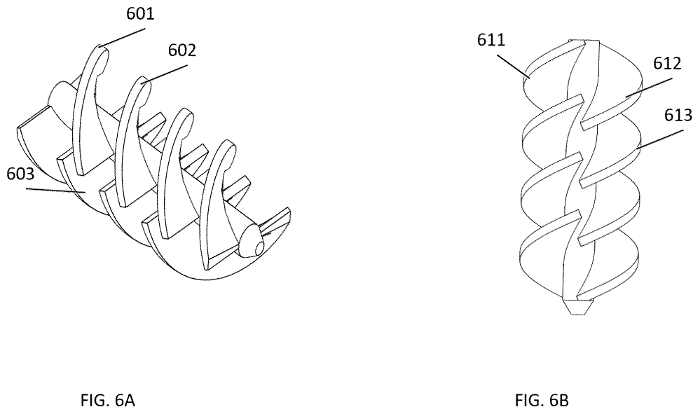

FIG. 6A shows a perspective view of an exemplary mixer body according to some embodiments. As shown, the mixer body may comprise a number of blades 601, 602, and 603, wherein blades 601 and 602 may be parallel to one another, while baffles 601 and 603 may not be parallel.

FIG. 6B shows a side view of another exemplary mixer body according to some embodiments. The mixer body of a static mixer shown in FIG. 6B may comprise a plurality of blades 611, 612, and 613, wherein blades 611 and 612 may be parallel and may be attached to the central shaft. In some embodiments, blades 613 may be located on a side opposite to blades 611 and 612.

FIG. 7A shows a perspective view of an exemplary depiction of a mixer body of a static mixer with channels. The mixer body may comprise a disc 701, a baffle 702, and/or a blade 703. A disc may comprise slots 704, 705. A slot 704 may be formed on a disc closer to a central shaft 706 of the mixer body. A slot 705 may be formed on an outer edge of a disc. The one or more fluid components in a mixer body may flow through one or more channels formed by a plurality of discs 704 comprising at least one slot. The baffle 702 or a blade 703 may further agitate the fluid components to generate a multi-component fluid mixture. In some embodiments, the baffle 702 and blade 703 generate a local turbulent flow that can generate rotational circulations within the mixing fluid to further mix the multi-component fluid. For example, the multi-component fluid may mix radially around the fluid's hydraulic centers. FIG. 7B shows a side view of a depiction of a similar mixer body. In some embodiments, discs 707 and 708 may be parallel to one another but not parallel to a disc 709. The discs 707, 708, or 709 may be configured to be perpendicular to an axis along the length of the mixer body (e.g., axis parallel to the central shaft 706), or they can be configured at an angle with respect a longitudinal axis of the mixer body central shaft. In some embodiments, for the mixer body examples depicted in FIGS. 7A-B, the angle between a disc (e.g., 707, 708, or 709) and a central shaft 706 may be about 1°, 2°, 3°, 4°, 5°, 6°, 7°, 8°, 9°, 10°, 15°, 20°, 25°, 30°, 35°, 40°, 45°, 50°, 60°, 70°, 80°, to about 90°.

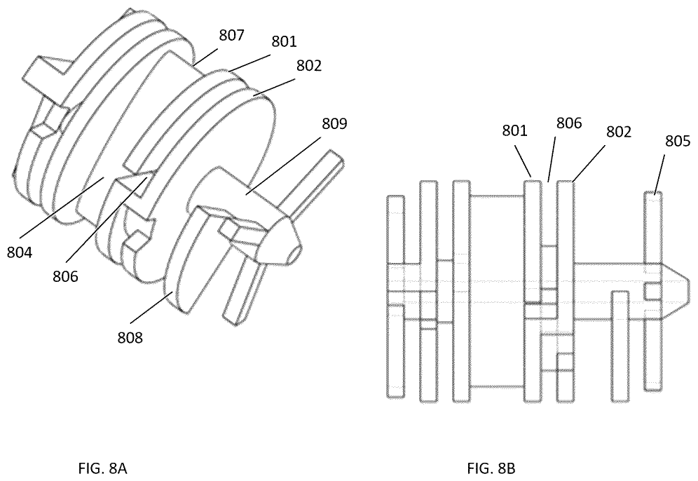

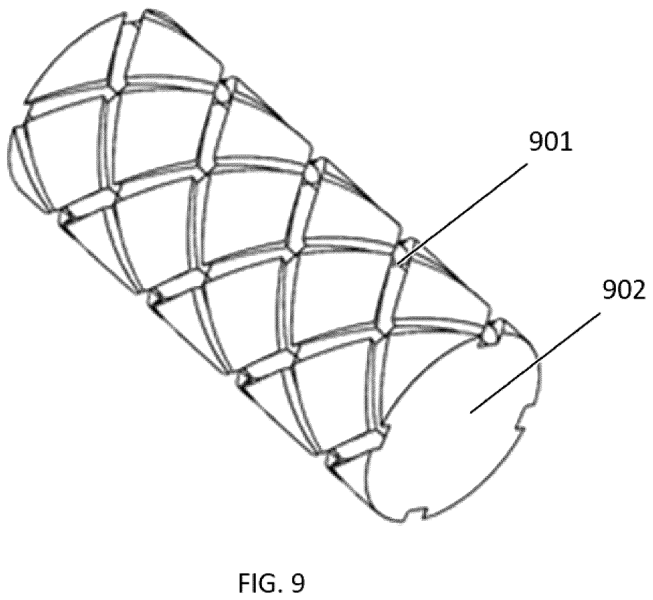

FIG. 8A shows another example of an inline mixer body with channels. The mixer may comprise a disc 801 or 802, and a baffle 804. The discs 801 and 802 may be configured to form a channel 806, which may be confined by 801 and 802. The two or more fluids in the mixer may flow through the channel 806 forcing the fluid components to mix. The fluid components may travel around the blade or baffle 804 that may mix the fluids by, for example, generating a sheer stress or a turbulent flow at an edge 807 of the baffle 804. In some embodiments, the fluid components may travel in a turbulent flow regime. In some embodiments, the fluid components may be a shear thinning fluid and may experience reduced viscosity as they travel in a turbulent flow regime. The mixer body may also comprise a baffle 808. In some embodiments, the baffle 808 may span a circular arc of about 1° to about 359°. The baffle 808 may comprise an arc of about 80° to about 90°. FIG. 8B shows a side view of the exemplary mixer body shown in FIG. 8A. Discs 801 and 802 may be parallel to one another. The channel 806 formed by 801 and 802 can have a depth of about 0.1 mm to about 50 mm. In some embodiments, the channel has a depth of about 0.1 mm to about 50 mm. In some embodiments, the channel has a depth of about 0.1 mm to about 0.2 mm, about 0.1 mm to about 0.3 mm, about 0.1 mm to about 0.4 mm, about 0.1 mm to about 0.5 mm, about 0.1 mm to about 1 mm, about 0.1 mm to about 5 mm, about 0.1 mm to about 10 mm, about 0.1 mm to about 20 mm, about 0.1 mm to about 30 mm, about 0.1 mm to about 40 mm, about 0.1 mm to about 50 mm, about 0.2 mm to about 0.3 mm, about 0.2 mm to about 0.4 mm, about 0.2 mm to about 0.5 mm, about 0.2 mm to about 1 mm, about 0.2 mm to about 5 mm, about 0.2 mm to about 10 mm, about 0.2 mm to about 20 mm, about 0.2 mm to about 30 mm, about 0.2 mm to about 40 mm, about 0.2 mm to about 50 mm, about 0.3 mm to about 0.4 mm, about 0.3 mm to about 0.5 mm, about 0.3 mm to about 1 mm, about 0.3 mm to about 5 mm, about 0.3 mm to about 10 mm, about 0.3 mm to about 20 mm, about 0.3 mm to about 30 mm, about 0.3 mm to about 40 mm, about 0.3 mm to about 50 mm, about 0.4 mm to about 0.5 mm, about 0.4 mm to about 1 mm, about 0.4 mm to about 5 mm, about 0.4 mm to about 10 mm, about 0.4 mm to about 20 mm, about 0.4 mm to about 30 mm, about 0.4 mm to about 40 mm, about 0.4 mm to about 50 mm, about 0.5 mm to about 1 mm, about 0.5 mm to about 5 mm, about 0.5 mm to about 10 mm, about 0.5 mm to about 20 mm, about 0.5 mm to about 30 mm, about 0.5 mm to about 40 mm, about 0.5 mm to about 50 mm, about 1 mm to about 5 mm, about 1 mm to about 10 mm, about 1 mm to about 20 mm, about 1 mm to about 30 mm, about 1 mm to about 40 mm, about 1 mm to about 50 mm, about 5 mm to about 10 mm, about 5 mm to about 20 mm, about 5 mm to about 30 mm, about 5 mm to about 40 mm, about 5 mm to about 50 mm, about 10 mm to about 20 mm, about 10 mm to about 30 mm, about 10 mm to about 40 mm, about 10 mm to about 50 mm, about 20 mm to about 30 mm, about 20 mm to about 40 mm, about 20 mm to about 50 mm, about 30 mm to about 40 mm, about 30 mm to about 50 mm, or about 40 mm to about 50 mm. In some embodiments, the channel has a depth of about 0.1 mm, about 0.2 mm, about 0.3 mm, about 0.4 mm, about 0.5 mm, about 1 mm, about 5 mm, about 10 mm, about 20 mm, about 30 mm, about 40 mm, or about 50 mm. In some embodiments, the channel has a depth of at least about 0.1 mm, about 0.2 mm, about 0.3 mm, about 0.4 mm, about 0.5 mm, about 1 mm, about 5 mm, about 10 mm, about 20 mm, about 30 mm, or about 40 mm. In some embodiments, the channel has a depth of at most about 0.2 mm, about 0.3 mm, about 0.4 mm, about 0.5 mm, about 1 mm, about 5 mm, about 10 mm, about 20 mm, about 30 mm, about 40 mm, or about 50 mm. The mixer body may also comprise a fin 805 to further mix the two or more fluids in the mixer body. The discs, fins, baffles, or blades in the mixer body may be configured to be perpendicular to an axis of the mixer body (e.g., axis parallel to the central shaft 809), or they can be configured at an angle with respect to the axis of the mixer body (e.g., axis parallel to the central shaft 809).

FIG. 9 shows an example of a mixer body comprising a series of channels having a plurality of intersection points 901. The channels may be formed on an outer surface of a central structure 902 (e.g., a central shaft). The channels may have a depth of about 0.1 millimeter (mm) to about 6 mm. The channels may be formed at a variety of angles.

### Steering Element

In some embodiments, the tube (e.g., a multi-lumen tube) may comprise a steering element. In some embodiments a steering element may be operably coupled to the multi-lumen tube. A steering element may provide the operator with a means of controlling the projection of a tube and/or a nozzle coupled to a tube. A steering element may orient the direction of the tube and/or a multi-component mixture delivery. In some embodiments, a user (e.g., a surgeon, an operator, a medical professional, a medical technician) may use the steering element to steer the direction of the delivery of a fluid being dispersed by the spraying device. The steering element may be handled by one hand or by two hands. In some embodiments, the steering may be controlled by a robotic arm. In some embodiments, a steering element may be configured to orient the multi-component mixture delivery from about 0° (i.e. 0° bend or deflection) to about 90° relative to the longitudinal axis of the tube (e.g., an axis from a distal end to a proximal end of the tube). The steering element may be configured to orient the multi-component mixture delivery from about 0°, 1°, 2°, 3°, 4°, 5°, 6°, 7°, 8°, 9°, 10°, 15°, 20°, 25°, 30°, 35°, 40°, 45°, 50°, 60°, 70°, 80°, 90°, or more than 90° relative to the longitudinal axis of the multi-lumen tube. The multi-lumen tube or a flexible portion thereof may have a bend radius of about 0 cm to about 5 cm. The bend radius may be about 0 cm to about 1 cm, about 0 cm to about 2 cm, about 0 cm to about 3 cm, about 0 cm to about 4 cm, about 0 cm to about 5 cm, about 1 cm to about 2 cm, about 1 cm to about 3 cm, about 1 cm to about 4 cm, about 1 cm to about 5 cm, about 2 cm to about 3 cm, about 2 cm to about 4 cm, about 2 cm to about 5 cm, about 3 cm to about 4 cm, about 3 cm to about 5 cm, or about 4 cm to about 5 cm.

FIG. 26A shows an example of a steering element. In some embodiments, a steering element comprises a rigid tube 2605, wherein at least a portion of the multi-lumen tube 2601 (also 102 from FIG. 1) may be disposed within the rigid tube 2605. In some embodiments, at least a portion of the multi-lumen tube may be configured to bend or coil. In some embodiments, the rigid tube 2605 slides over the multi-lumen tube to straighten said multi-lumen tube. As described further below, the rigid tube can control the angle at which the nozzle may be oriented relative to a longitudinal axis of the multi-lumen tube (or longitudinal axis of the rigid tube) based on how much of the distal portion of the multi-lumen tube (e.g., 2603) may be disposed within the rigid tube (i.e. based on how much of the distal portion of the multi-lumen tube may not be disposed within the rigid tube). In some embodiments, the rigid tube may be built onto the multi-lumen tube. In some embodiments, the rigid tube may be coupled to the multi-lumen tube as a separate component.

The rigid tube may be configured to deliver a force to the coiled or bent portion 2603 (similar to Ref. Char. 110 from FIG. 1) of the multi-lumen tube 2601 (which may be similar to ref char. 102 from FIG. 1). In some embodiments, the rigid tube surrounds (or wraps around) the multi-lumen tube. In some embodiments, the multi-lumen tube 2601 and/or the bent portion thereof 2603 comprises a flexible polymeric tube capable of elastic deformation, positioned inside of the rigid tube 2605 of the steering element. The rigid tube 2605 may slide over the multi-lumen tube 2601 or the bent portion thereof 2603 causing it to straighten. When the rigid tube 2605 may be retracted, the multi-lumen tube 2601 or the bent portion thereof 2603 may recoil into a bent configuration. The steering element comprising the rigid tube may be configured to change a bend angle in the bent portion 2603, as described herein, to steer the portion 2604 comprising a nozzle. The portion 2604 may comprise a mixer and the nozzle (e.g., see FIGS. 4A-4C). The multi-lumen tube 2601 may be connected to a fluid component source(e.g., a fluid container) to receive a component of the multi-component fluid via connector 2607. The multi-lumen tube 2601 may be connected to a dispersant source (e.g., a container, or a gas compressor) receive a dispersant via connector 2606.

A position or orientation of the nozzle may be controlled or adjusted by steering the steering element (e.g., retracting the rigid tube). FIGS. 26B-26F illustrate examples of the rigid tube 2605 straightening a portion 2603 of the multi-lumen tube, thereby changing a position or orientation of the portion 2604 comprising the nozzle. FIG. 26B shows a neutral position of a portion 2603 of the multi-lumen tube that may not be disposed within the steering element 2605. In some embodiments, the portion 2603 may be part of the multi-lumen tube, and may be bent or curved when not disposed within the rigid tube. In some embodiments, the entire multi-lumen tube may be configured to be curved or bent when not disposed within a rigid tube. In some embodiments, the portion 2603 may be a separate component from the multi-lumen tube that may be coupled to and distal to the multi-lumen tube 2601, and couple to and proximal to the portion 2604. The rigid tube 2605 of the steering element may be fully retracted as shown in FIG. 26B. The rigid tube 2605 of the steering element may be advanced over the multi-lumen tube 2601 to apply force over a portion of the portion 2603 reducing the bend angle as shown in FIGS. 26C-26E. The rigid tube 2605 of the steering element may be advanced over the multi-lumen tube 2601 to apply force over the entire portion 2603 reducing the bend angle to about 0° as shown in FIG. 26F. The nozzle can be therefore steered by changing the bend angle of the bent portion 2603 which can change the position or orientation of the portion 2604 comprising the nozzle.

A multi-lumen tube or a portion thereof (e.g., the bent portion 2603) may comprise polystyrene, polyvinyl chloride, polychlorotrifluoroethylene, polyethylene, Bakelite, Kevlar, Twaron, Mylar, Neoprene, Nylon, Nomex, Orlon, Rislan, Technora, Teflon, Ultem, Vectran, Viton, Zylon, polysiloxane, polyphosphazene, polythene, polypropene, melamine, lexan, vinyl rubber, polyacrylonitrile, copolyamid, acrylonitrile-butadiene-styrene, allyl resin, cellulosic, epoxy, ethylene vinyl alcohol, floroplastics, ionomer, phenol-formaldehyde plastic, polyacetal, polyacrylate, polyacrylonitrile, polyamide-imide, polyaryletherketone, polybutadiene, polybutylene, polycarbonate, polydicyclopentadiene, polyektone, polyester, polyetheretherketone, polyetherimide, polyethersulfone, polyethylenechlorinate, polypethylpentene, polyphenylene oxide, polyphenylene sulfide, poly ethylene glycol, polylactic acid, poly lactic co-glycolic acid, polyphenylene sulfide, polyethersulfone, polyphthalamide, polysulfone, polyurethane, polyvinylidene chloride, silicone, thermoplastic elastomers, polyethylene terephthalate, rubber, plastics, elastics, elastomers, organic polymers, inorganic polymers, natural polymers, thermosets, thermoplastics, starch, polymethyl methacrylate, aramids, rayon, polytetrafluoroethylene, poly styrene-butadiene-styrene, semi-crystalline polymers, amorphous polymers, copper, iron, aluminum, solver, gold, lead, zinc, nickel, platinum, tin, titanium, cobalt, chromium, tungsten, molybdenum, palladium, vanadium, cadmium, lithium, rhodium, zirconium, niobium, tantalum, gallium, beryllium, barium, strontium, radium, steel, aluminum, silicon carbide, titanium carbide, tungsten carbide, barium titanate, boron carbide, ferrite, strontium titanate, titanium oxide, carbides, nitrides, silicon carbide, silicon nitride, silicon dioxide, aluminum oxide, hydrous aluminum silicate, glass, mullite, cristobalite, calcium oxide, Pyrex, electroceramics, alumina, zirconia, boride, silicide, silicate, pyroceram, soda-lime, spinel, diamond, carbon fiber, carbon, or glass fiber. In some embodiments, an outer surface of the multi-lumen tube or a portion thereof or an inner surface of the rigid tube or both comprise a surface coating that reduces friction. The coating may comprise a lubricating agent. The coating may comprise Everlube®, PTFE®, MoS2®, Zinc Rich®, Impingement®, Teflon®, Nonstick®, Primers®, PROCOAT 100®, or other solid or liquid lubricants. The coating may comprise parylene.

In some embodiments, the rigid tube of the steering element may comprise polystyrene, polyethylene, polyvinyl chloride, polychlorotrifluoroethylene, Bakelite, Kevlar, Twaron, Mylar, Neoprene, Nylon, Nomex, Orlon, Rislan, Technora, Teflon, Ultem, Vectran, Viton, Zylon, polysiloxane, polyphosphazene, polythene, polypropene, melamine, lexan, vinyl rubber, polyacrylonitrile, copolyamid, acrylonitrile-butadiene-styrene, allyl resin, cellulosic, epoxy, ethylene vinyl alcohol, floroplastics, ionomer, phenol-formaldehyde plastic, polyacetal, polyacrylate, polyacrylonitrile, polyamide-imide, polyaryletherketone, polybutadiene, polybutylene, polycarbonate, polydicyclopentadiene, polyektone, polyester, polyetheretherketone, polyetherimide, polyethersulfone, polyethylenechlorinate, polypethylpentene, polyphenylene oxide, polyphenylene sulfide, poly ethylene glycol, polylactic acid, poly lactic co-glycolic acid, polyphenylene sulfide, polyethersulfone, polyphthalamide, polysulfone, polyurethane, polyvinylidene chloride, silicone, thermoplastic elastomers, polyethylene terephthalate, rubber, plastics, elastics, elastomers, organic polymers, inorganic polymers, natural polymers, thermosets, thermoplastics, starch, polymethyl methacrylate, aramids, rayon, polytetrafluoroethylene, poly styrene-butadiene-styrene, semi-crystalline polymers, amorphous polymers, copper, iron, aluminum, silver, gold, lead, zinc, nickel, platinum, tin, titanium, cobalt, chromium, tungsten, molybdenum, palladium, vanadium, cadmium, lithium, rhodium, zirconium, niobium, tantalum, gallium, beryllium, barium, strontium, radium, steel, aluminum, silicon carbide, titanium carbide, tungsten carbide, barium titanate, boron carbide, ferrite, strontium titanate, titanium oxide, carbides, nitrides, silicon carbide, silicon nitride, silicon dioxide, aluminum oxide, hydrous aluminum silicate, glass, mullite, cristobalite, calcium oxide, Pyrex, electroceramics, alumina, zirconia, boride, silicide, silicate, pyroceram, soda-lime, spinel, diamond, carbon fiber, carbon, or glass fiber.

In some embodiments, a rigid tube may comprise a metal. A metallic rigid tube may be capable of elastic deformation. The metallic rigid tube may be placed along or over the multi-lumen tube or a portion thereof (e.g., the bent portion 2603) to apply force to change a bending or coiling of the bent portion 2603. A metallic rigid tube may comprise lithium, beryllium, sodium, magnesium, aluminum, potassium, calcium, scandium, titanium, vanadium, chromium, manganese, iron, cobalt, nickel, copper, zinc, gallium, rubidium, strontium, yttrium, zirconium, niobium, molybdenum, technetium, ruthenium, rhodium palladium silver, cadmium, indium, tin, cesium, barium, lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold, mercury, thallium, lead, bismuth, polonium, francium, radium, actinium, thorium, protactinium, uranium, neptunium, plutonium, americium, curium, berkelium, californium, nobelium, or fermium.

In some embodiments, the steering element may comprise malleable material (e.g., metallic) capable of elastic deformation. The elastically deformable steering element may be disposed along the tube to enable the bending or coiling properties to steer the spraying device. The elastically deformable steering element may comprise a bar or casing (e.g., a tube shell) disposed or wrapped around the multi-lumen tube along the longitudinal axis of the multi-lumen tube.

In some embodiments, a strip of electro-stimuli responsive material may be placed on or around the steering element (e.g., the rigid tube) such that an operator can manipulate the direction of the nozzle via electric or electronic control. A electro-stimuli responsive material may comprise an electroactive polymer, an electro-sensitive hydrogel, a semiconductor, or conductor which may comprise poly(acrylic acid), hydroxyethyl methacrylate, poly (acrylamide), poly (methacrylic acid), poly (vinyl alcohol), poly (N-isopropylacrylamide), polycaprolactone, chitosan, hydroxybutl acrylate, poly(2-acrylamido-2-methylpropanesulfonic acid), 4-vinylbenzenesulfonate, polyacrylonitrile, acrylamide, graphene oxide, iron oxide, polyethylene glycol, poly(ethylene glycol) dimethacrylate, germanium, silicon, gallium arsenide, silicon carbide, gallium nitride, gallium phosphide, cadmium sulphide, lead sulphide, boron, carbon, nitrogen, aluminum, phosphorus, arsenic, indium, tin, antimony, gold, silver, copper, diamond, iron, steel, brass, bronze, mercury, or graphite. A nozzle may be moved in a step-wise fashion, fluidly, or in a varied motion. In some embodiments, the steering element may comprise a series of cables configured to move the rigid tube, as described herein.

### Nozzle

In some embodiments, the spray device comprises a nozzle. In some embodiments, a nozzle may be circular, cylindrical, or conical. In some embodiments, the nozzle 410 may be embedded within the housing 407 of a mixer chamber, as shown in FIG. 4C. A nozzle may comprise biocompatible material. The material may be rigid. The material used to form the nozzle may be amenable to be coupled to other parts of the spraying device (e.g., mixer, mixer housing, or tube) via adhesive, epoxy, ultrasonic welding, or other coupling methods mentioned herein. Non-limiting examples of nozzle materials may comprise polycarbonate, acrylic, polyethylene, polystyrene, poly ether ether ketone (PEEK), stainless steel, or titanium. A nozzle may have a geometry such as a circle, rectangle, or ellipse. The nozzle may be used to further mix the multi-component mixture, aerosolize the mixture, or disperse the mixture. In some embodiments, the nozzle may be configured to deliver a mixture in the form of particles or a jet of fluid. The geometry of the nozzle may impact a shape or size of the particles, or a velocity of the particles or the jet of fluids being dispersed. In some embodiments, the inlet of the nozzle and/or the outlet of the nozzle may have a circular, rectangular, elliptical, or any polygamical geometry. In some embodiments, the inlet of the nozzle and/or the outlet of the nozzle comprises a complex 3-Dimensional shape, or a more complex geometry, that incorporates features to destabilize the flow therein and induce aerosolization. A particle formed by the nozzle may have a diameter of about 10 μm to about 500 μm. In some cases, the particle diameter may be about 10 μm to about 20 μm, about 10 μm to about 50 μm, about 10 μm to about 100 μm, about 10 μm to about 200 μm, about 10 μm to about 300 μm, about 10 μm to about 400 μm, about 10 μm to about 500 μm, about 20 p.m to about 50 μm, about 20 μm to about 100 μm, about 20 μm to about 200 μm, about 20 μm to about 300 μm, about 20 μm to about 400 μm, about 20 μm to about 500 μm, about 50 μm to about 100 μm, about 50 μm to about 200 μm, about 50 μm to about 300 μm, about 50 μm to about 400 pm, about 50 μm to about 500 μm, about 100 μm to about 200 μm, about 100 μm to about 300 μm, about 100 μm to about 400 μm, about 100 μm to about 500 μm, about 200 μm to about 300 μm, about 200 μm to about 400 pm, about 200 μm to about 500 μm, about 300 μm to about 400 μm, about 300 μm to about 500 μm, or about 400 μm to about 500 μm. In some embodiments, the particles or the jet of fluid generated or dispersed from the nozzle may have a velocity of about 7 millimeter per second (mm/s) to about 2000 meters per second (m/s). In some cases, the velocity may be about 7 mm/s to about 20 mm/s, about 7 mm/s to about 50 mm/s, about 7 mm/s to about 100 mm/s, about 7 mm/s to about 200 mm/s, about 7 mm/s to about 500 mm/s, about 7 mm/s to about 1,000 mm/s, about 7 mm/s to about 1,500 mm/s, about 7 mm/s to about 2,000 mm/s, about 20 mm/s to about 50 mm/s, about 20 mm/s to about 100 mm/s, about 20 mm/s to about 200 mm/s, about 20 mm/s to about 500 mm/s, about 20 mm/s to about 1,000 mm/s, about 20 mm/s to about 1,500 mm/s, about 20 mm/s to about 2,000 mm/s, about 50 mm/s to about 100 mm/s, about 50 mm/s to about 200 mm/s, about 50 mm/s to about 500 mm/s, about 50 mm/s to about 1,000 mm/s, about 50 mm/s to about 1,500 mm/s, about 50 mm/s to about 2,000 mm/s, about 100 mm/s to about 200 mm/s, about 100 mm/s to about 500 mm/s, about 100 mm/s to about 1,000 mm/s, about 100 mm/s to about 1,500 mm/s, about 100 mm/s to about 2,000 mm/s, about 200 mm/s to about 500 mm/s, about 200 mm/s to about 1,000 mm/s, about 200 mm/s to about 1,500 mm/s, about 200 mm/s to about 2,000 mm/s, about 500 mm/s to about 1,000 mm/s, about 500 mm/s to about 1,500 mm/s, about 500 mm/s to about 2,000 mm/s, about 1,000 mm/s to about 1,500 mm/s, about 1,000 mm/s to about 2,000 mm/s, or about 1,500 mm/s to about 2,000 mm/s.

A jet of fluids may disperse into particles at a distance of at most about 5 centimeters (cm) from a nozzle outlet. In some cases, the jet of fluids may disperse into particles at a distance of about 0.5 cm to about 1 cm, about 0.5 cm to about 1.5 cm, about 0.5 cm to about 2 cm, about 0.5 cm to about 2.5 cm, about 0.5 cm to about 3 cm, about 0.5 cm to about 3.5 cm, about 0.5 cm to about 4 cm, about 0.5 cm to about 4.5 cm, about 1 cm to about 1.5 cm, about 1 cm to about 2 cm, about 1 cm to about 2.5 cm, about 1 cm to about 3 cm, about 1 cm to about 3.5 cm, about 1 cm to about 4 cm, about 1 cm to about 4.5 cm, about 1.5 cm to about 2 cm, about 1.5 cm to about 2.5 cm, about 1.5 cm to about 3 cm, about 1.5 cm to about 3.5 cm, about 1.5 cm to about 4 cm, about 1.5 cm to about 4.5 cm, about 2 cm to about 2.5 cm, about 2 cm to about 3 cm, about 2 cm to about 3.5 cm, about 2 cm to about 4 cm, about 2 cm to about 4.5 cm, about 2.5 cm to about 3 cm, about 2.5 cm to about 3.5 cm, about 2.5 cm to about 4 cm, about 2.5 cm to about 4.5 cm, about 3 cm to about 3.5 cm, about 3 cm to about 4 cm, about 3 cm to about 4.5 cm, about 3.5 cm to about 4 cm, about 3.5 cm to about 4.5 cm, or about 4 cm to about 4.5 cm from the nozzle. In some cases, the jet of fluids may disperse into particles at a distance of about 0.5 cm, about 1 cm, about 1.5 cm, about 2 cm, about 2.5 cm, about 3 cm, about 3.5 cm, about 4 cm, or about 4.5 cm from the nozzle. In some embodiments, a nozzle may comprise a plurality of nozzles. The plurality of nozzles may generate smaller particles and/or aerosolize at a shorter distant from a nozzle.

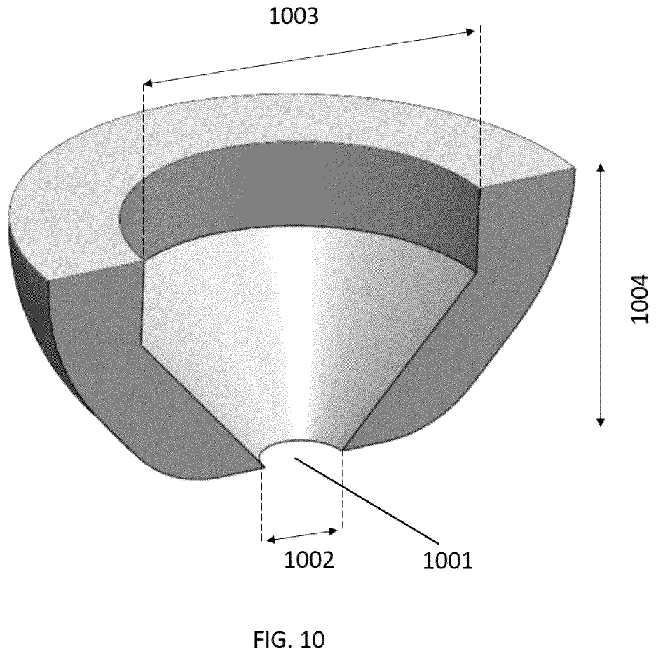

FIG. 10 shows a cross-sectional view of an exemplary depiction of a circular nozzle. In some embodiments, a nozzle comprises an inlet, a nozzle body, and an outlet. The nozzle may comprise a circular outlet 1001. The nozzle may comprise a geometric feature comprising an inlet diameter 1003, an outlet diameter 1002, and/or a nozzle body length 1004. The nozzle may have an outlet diameter 1002. In some embodiments, the outlet diameter of the nozzle may be about 0.1 millimeter (mm) to about 4 mm. In some embodiments, the inlet 1003 may have a diameter of about 0.1 mm to about 4 mm. In some embodiments, the nozzle body may have a length 1004 of about 0.1 mm to about 10 mm. In some embodiments, the nozzle tapers outwards from the nozzle inlet to the nozzle outlet (e.g., nozzle inlet diameter may be smaller than nozzle outlet diameter). In some embodiments, the nozzle tapers inwards from the nozzle inlet to the nozzle outlet (e.g., nozzle inlet diameter may be larger than nozzle outlet diameter).

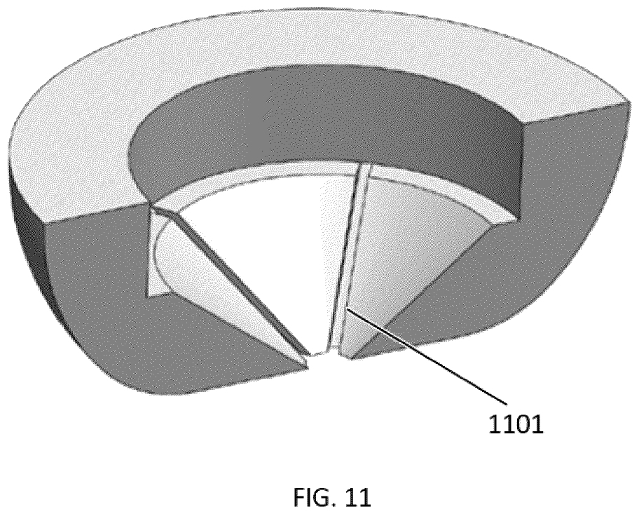

FIG. 11 shows a cross-sectional view of an exemplary depiction of a nozzle comprising a microchannel. The nozzle may comprise a structural or geometrical feature to facilitate aerosolization of a fluid being dispersed form the nozzle. The structural feature may comprise a microchannel 1101. In some embodiments, the microchannel 1101 may be a microtube. In some embodiments, a ratio of a depth of the microchannel 1101 to an inlet or outlet diameter of the nozzle may be about 0.001 to about 0.5. The nozzle may comprise 1, 2, 3, 4, 5, 6, 7, 8, 9, 10, or more microtubes.

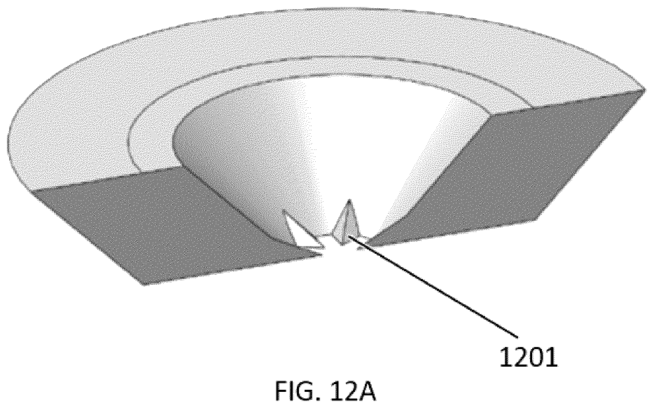

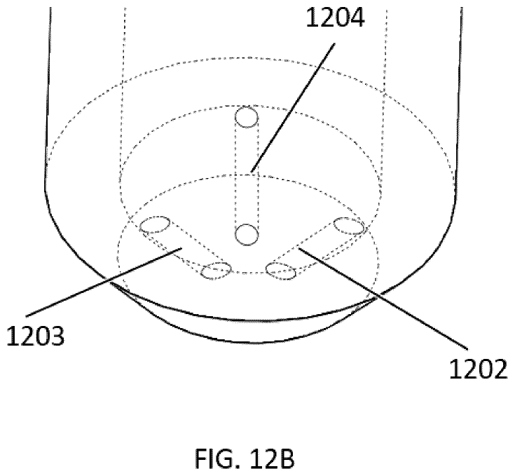

FIG. 12A shows a cross-sectional view of an exemplary depiction of a nozzle with a micro-protrusion. The micro-protrusion 1201 may comprise a triangular shape. In some embodiments, the micro-protrusion protrudes about 0.05 mm to about 4 mm. The micro-protrusion may facilitate aerosolization of a fluid being dispersed form the nozzle. The nozzle may comprise 1, 2, 3, 4, 5, 6, 7, 8, 9, 10, or more micro-protrusion.

FIG. 12B illustrates an example of a nozzle comprising a plurality of smaller nozzles or channels 1202/1203/1204. The plurality of smaller nozzles or channels 1202/1203/1204 may create multiple smaller streams of the multi-component fluid. The multi-component fluid may aerosolize on impact with each other when the fluid may be delivered out of plurality of smaller nozzles or channels 1202/1203/1204.

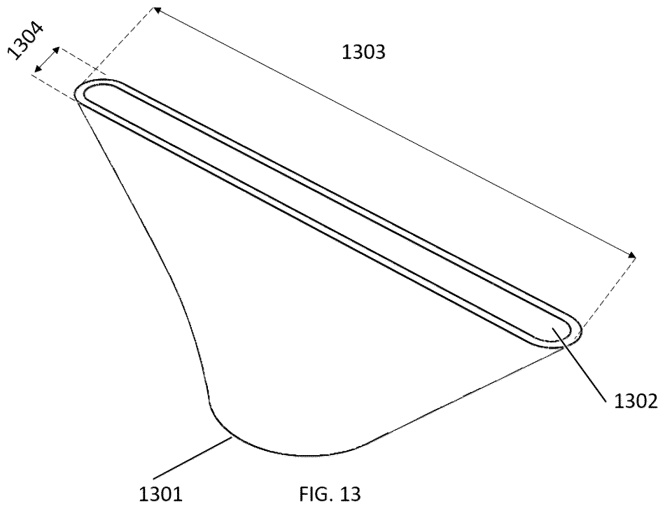

FIG. 13 shows an exemplary depiction of an applicator tip according to some embodiments. The applicator tip may comprise a first end 1301, and a second end 1302. The applicator tip may be coupled to a nozzle from 1301. In some embodiments, the applicator tip may be used as an alternate to a nozzle. In some embodiments the applicator tip may be in fluid communication with the mixer and gas dispersant passageway. In some embodiments, the applicator tip comprises a similar material as disclosed herein for a nozzle. The applicator tip may be used to dispense a thin film of a multicomponent fluid from 1302. The end 1302 may have a thickness 1304 and a width 1303. In some embodiments, the width about 2 mm to about 50 mm. In some embodiments, the width may be about 2 mm to about 4 mm, about 2 mm to about 6 mm, about 2 mm to about 8 mm, about 2 mm to about 10 mm, about 2 mm to about 12 mm, about 2 mm to about 15 mm, about 2 mm to about 20 mm, about 2 mm to about 30 mm, about 2 mm to about 50 mm, about 4 mm to about 6 mm, about 4 mm to about 8 mm, about 4 mm to about 10 mm, about 4 mm to about 12 mm, about 4 mm to about 15 mm, about 4 mm to about 20 mm, about 4 mm to about 30 mm, about 4 mm to about 50 mm, about 6 mm to about 8 mm, about 6 mm to about 10 mm, about 6 mm to about 12 mm, about 6 mm to about 15 mm, about 6 mm to about 20 mm, about 6 mm to about 30 mm, about 6 mm to about 50 mm, about 8 mm to about 10 mm, about 8 mm to about 12 mm, about 8 mm to about 15 mm, about 8 mm to about 20 mm, about 8 mm to about 30 mm, about 8 mm to about 50 mm, about 10 mm to about 12 mm, about 10 mm to about 15 mm, about 10 mm to about 20 mm, about 10 mm to about 30 mm, about 10 mm to about 50 mm, about 12 mm to about 15 mm, about 12 mm to about 20 mm, about 12 mm to about 30 mm, about 12 mm to about 50 mm, about 15 mm to about 20 mm, about 15 mm to about 30 mm, about 15 mm to about 50 mm, about 20 mm to about 30 mm, about 20 mm to about 50 mm, or about 30 mm to about 50 mm. In some embodiments, the width may be about 2 mm, about 4 mm, about 6 mm, about 8 mm, about 10 mm, about 12 mm, about 15 mm, about 20 mm, about 30 mm, or about 50 mm. In some embodiments, the width may be at least about 2 mm, about 4 mm, about 6 mm, about 8 mm, about 10 mm, about 12 mm, about 15 mm, about 20 mm, or about 30 mm. In some embodiments, the width may be at most about 50 mm, about 30 mm, about 20 mm, about 15 mm, about 12 mm, about 10 mm, about 8 mm, about 6 mm, about 4 mm, about 2 mm, or less. In some embodiments, the applicator tip may be about 0.1 mm to about 2 mm thick (e.g., the thickness 1304).

### Aerosolization

In some embodiments, the spray device aerosolizes the mixture (e.g., multi-component fluid) prior to or during delivery from the spray device. Aerosolization may improve uniformity of the application of the multi-component fluid to an area of interest. In some embodiments, the dispersant (e.g., dispersant gas, as described herein) may facilitate aerosolization of the multi-component fluid. In some embodiments, the dispersant facilitates aerosolization by generating a pressure difference between the nozzle inlet and the nozzle outlet. In some embodiments, the dispersant may be provided to the nozzle via the dispersant passageway within the central shaft of the mixer body (as described herein). In some embodiment, the dispersant from the dispersant passageway contacts the multi-component fluid (e.g., formed via the mixer as described herein) prior to, within, or downstream the nozzle. In some embodiment, the dispersant from the dispersant passageway contacts the multi-component fluid (e.g., formed via the mixer as described herein) prior to, within, or downstream the nozzle outlet. The dispersant may be configured to carry particles (e.g., droplets) of the multi-component fluid after delivery from the nozzle outlet. In some embodiments, nozzle shape, or a geometrical feature which may protrude into or be cut out of the nozzle orifice may perturb the flow of the multicomponent fluid as it exits the nozzle orifice to facilitate aerosolization. The material to be sprayed may be sensitive to high shear stress. A mixer or a nozzle may be configured to mitigate, obviate, or induce shear stress by geometric features. In some embodiments, aerosolization may be facilitated by injection of a dispersant (e.g., a compressed gas). The multi-component fluid may be delivered through the nozzle outlet in form of a jet of fluid. In some embodiments, a dispersant can be delivered through the nozzle simultaneously with the multifluid delivery out of the nozzle outlet to form aerosols. A compressed gas may comprise carbon dioxide, oxygen, nitrogen, helium, atmospheric air, argon, neon, xenon, krypton, radon, acetylene, butane, ethylene, hydrogen, methylamine, vinyl chloride, nitrogen oxides, halogen gases such as chlorine and fluorine, acetylene,1,3-butadiene, methyl acetylene, tetrafluoroethylene or vinyl fluoride. In some embodiments, a gas may be injected concentrically with the multi-component fluid. In some embodiments a gas may be injected coaxially with the multi-component fluid stream. In some embodiments, a gas may be injected eccentrically with a multi-component fluid stream being delivered. In some embodiments, eccentrically injecting the gas with the multi-component fluid comprises injecting the gas proximal to the nozzle outlet but off-center. In some embodiments, a gas may be injected adjacent to a multi-component fluid stream.

### Exemplary Method

An example of using a device disclosed herein may be provided. During a surgical procedure in the abdomen or pelvis, access sites may be created, either by minimally invasive techniques including laparoscopic and robotic approaches or by traditional opened surgeries such as laparotomies. The surgeon inserts the appropriated instruments to perform the procedure. At the conclusion of the procedure all surgical tools and instruments may be withdrawn. At this point, the nozzle end of the delivery system described here could be inserted through the access site and guided to the site of the procedure by use of the steering mechanism. Upon pressing the button to spray, the dispenser will depress the plungers such that their contents will move through the tube where they will enter the mixer. In some embodiments, the dispenser will depress the plungers such that their contents will move through the tube where they will enter the mixer by way of a constant force spring. At the distal end of the mixer, they will exit through the nozzle with air assist to form small droplets which will gel on contact with the warm tissue. The surgeon will continue until the surfaces of the organs and abdominal wall may be coated. The delivery device can be withdrawn and surgical access sites closed.

### Computer Systems

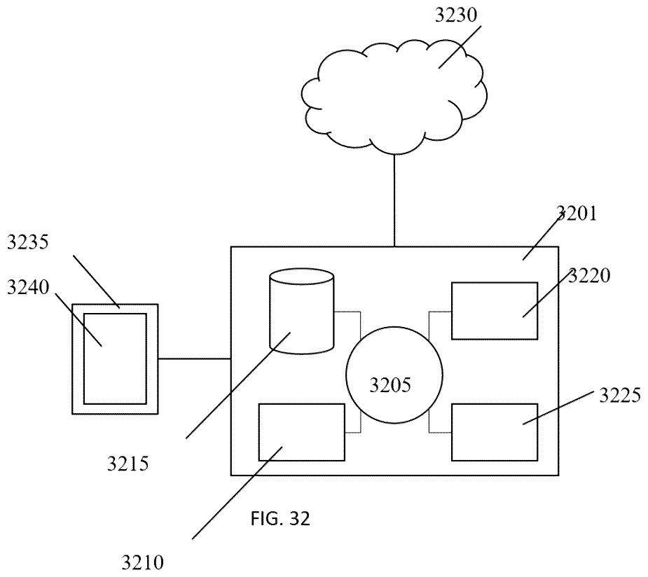

The present disclosure provides computer systems that may be programmed to implement methods of the disclosure. FIG. 32 shows a computer system 3201 that may be programmed or otherwise configured to perform the methods described herein. The computer system 3201 can regulate various aspects of the present disclosure, such as, for example, the automatic dispensing of one or more components of a multi-component mixture. The computer system 3201 can be an electronic device of a user or a computer system that may be remotely located with respect to the electronic device. The electronic device can be a mobile electronic device.

The computer system 3201 includes a central processing unit (CPU, also “processor” and “computer processor” herein) 3205, which can be a single core or multi core processor, or a plurality of processors for parallel processing. The computer system 3201 also includes memory or memory location 3210 (e.g., random-access memory, read-only memory, flash memory), electronic storage unit 3215 (e.g., hard disk), communication interface 3220 (e.g., network adapter) for communicating with one or more other systems, and peripheral devices 3225, such as cache, other memory, data storage and/or electronic display adapters. The memory 3210, storage unit 3215, interface 3220 and peripheral devices 3225 may be in communication with the CPU 3205 through a communication bus (solid lines), such as a motherboard. The storage unit 3215 can be a data storage unit (or data repository) for storing data. The computer system 3201 can be operatively coupled to a computer network (“network”) 3230 with the aid of the communication interface 3220. The network 3230 can be the Internet, an internet and/or extranet, or an intranet and/or extranet that may be in communication with the Internet. The network 3230 in some cases may be a telecommunication and/or data network. The network 3230 can include one or more computer servers, which can enable distributed computing, such as cloud computing. The network 3230, in some cases with the aid of the computer system 3201, can implement a peer-to-peer network, which may enable devices coupled to the computer system 3201 to behave as a client or a server.

The CPU 3205 can execute a sequence of machine-readable instructions, which can be embodied in a program or software. The instructions may be stored in a memory location, such as the memory 3210. The instructions can be directed to the CPU 3205, which can subsequently program or otherwise configure the CPU 3205 to implement methods of the present disclosure. Examples of operations performed by the CPU 3205 can include fetch, decode, execute, and writeback.

The CPU 3205 can be part of a circuit, such as an integrated circuit. One or more other components of the system 3201 can be included in the circuit. In some embodiments, the circuit may be an application specific integrated circuit (ASIC).

The storage unit 3215 can store files, such as drivers, libraries and saved programs. The storage unit 3215 can store user data, e.g., user preferences and user programs. The computer system 3201 in some cases can include one or more additional data storage units that may be external to the computer system 3201, such as located on a remote server that may be in communication with the computer system 3201 through an intranet or the Internet.

The computer system 3201 can communicate with one or more remote computer systems through the network 3230. For instance, the computer system 3201 can communicate with a remote computer system of a user (e.g., a cellphone, a portable computer, a laptop). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 3201 via the network 3230.

Methods as described herein can be implemented by way of machine (e.g., computer processor) executable code stored on an electronic storage location of the computer system 3201, such as, for example, on the memory 3210 or electronic storage unit 3215. The machine executable or machine-readable code can be provided in the form of software. During use, the code can be executed by the processor 3205. In some embodiments, the code can be retrieved from the storage unit 3215 and stored on the memory 3210 for ready access by the processor 3205. In some situations, the electronic storage unit 3215 can be precluded, and machine-executable instructions may be stored on memory 3210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 3201, can be embodied in programming. Various aspects of the technology may be thought of as “products” or “articles of manufacture” typically in the form of machine (or processor) executable code and/or associated data that may be carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 3201 can include or be in communication with an electronic display 3235 that comprises a user interface (UI) 3240, for example, driving the automated dispensing of one or more components of a multi-component mixture. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 3205. The algorithm can, for example, determine the flow rate of one or more components dispensed in a multi-component mixture.

### Fluid Dispersion Devices, Configurations, and Hydrogel Dispersion

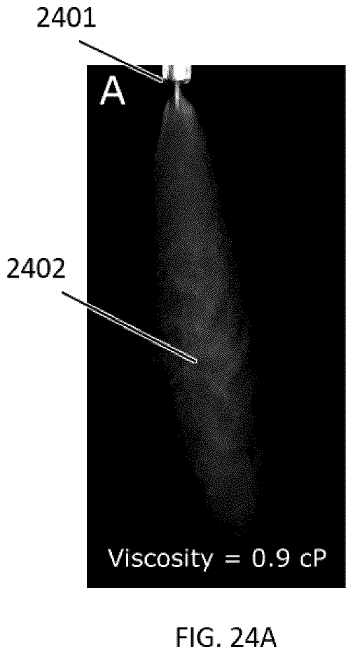

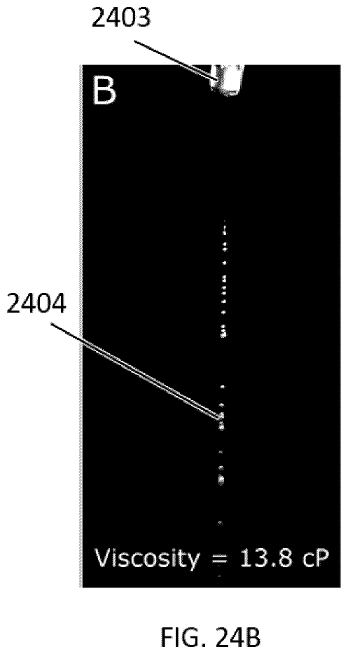

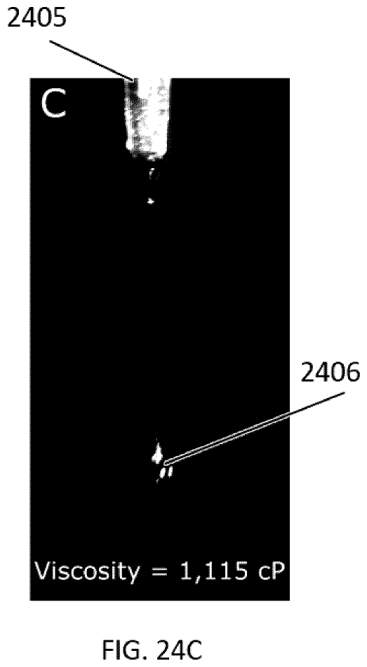

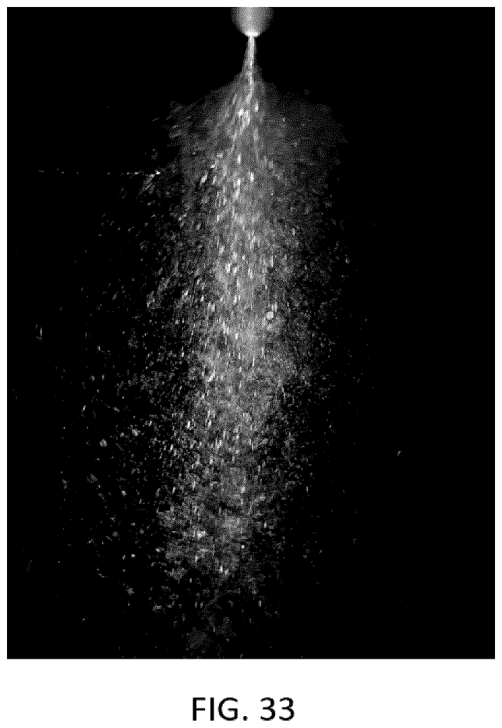

FIG. 33. Shows an image of an example of a fluid jet from exiting a nozzle with a gas assisted delivery system. The fluid dispersed from the nozzle may be the same fluid shown disbursed through the same nozzle as FIG. 24B, except that the nozzle of FIG. 33 has a gas assisted delivery. As can be observed, the gas assisted nozzle resulted in a significant increase in aerosolization of the high viscosity fluid, and would result in significantly increased coverage of tissue were it to be dispersed in vivo.

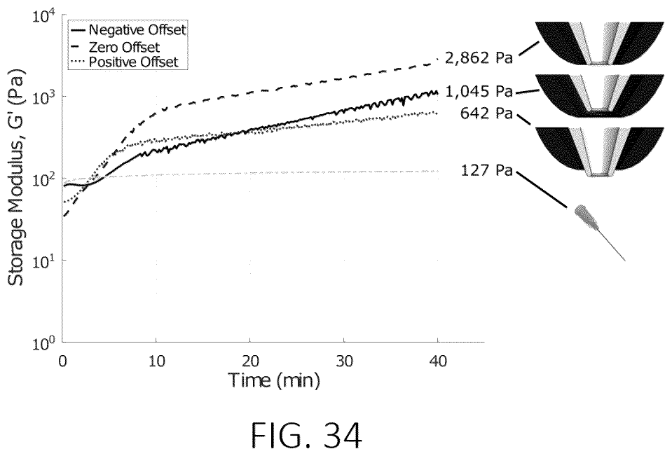

FIG. 34. Shows a graph of the storage modulus of a fluid material dispensed from nozzles of differing embodiments, with a negative offset nozzle, a 0 offset nozzle, and a positive offset nozzle. The stiffness of the gels resulting from injected, sprayed with a positive offset, sprayed with a negative offset, and sprayed with a positive offset are shown in ascending order. As can be observed from the graph, the gel sprayed from the nozzle with zero offset relative to the point of dispersion obtained the greatest stiffness of 2,862 Pa; the gel sprayed from the nozzle with a negative offset obtained a stiffness of 1,045 Pa; the gel sprayed from the nozzle with a positive offset obtained a stiffness of 642 Pa; and the gel that was only injected obtained a stiffness of only 127 Pa. In some embodiments, a nozzle with a 0 offset from the point of dispersion may result in gel with a higher stiffness and may be ideal for preventing adhesions in vivo.

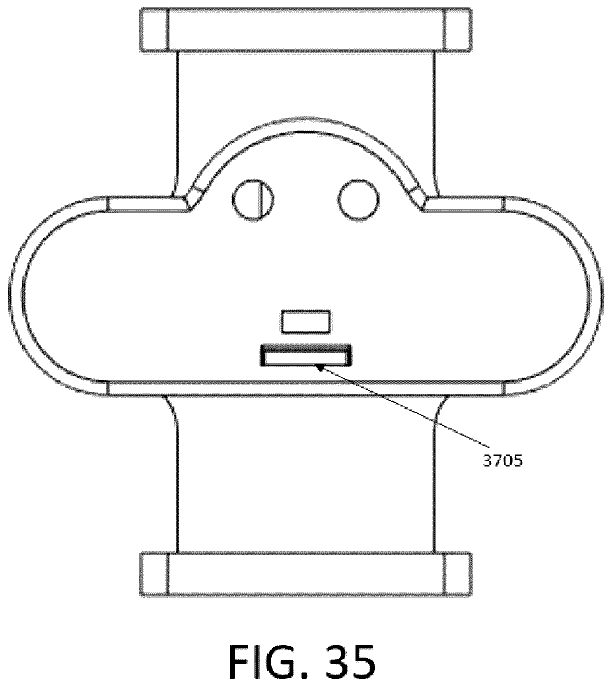

FIGS. 35-39 shows various views of a fluid dispersion device of some embodiments.

FIG. 35 shows a rear view of a fluid dispersion device, and shown is the constant force spring attachment slot 3705.

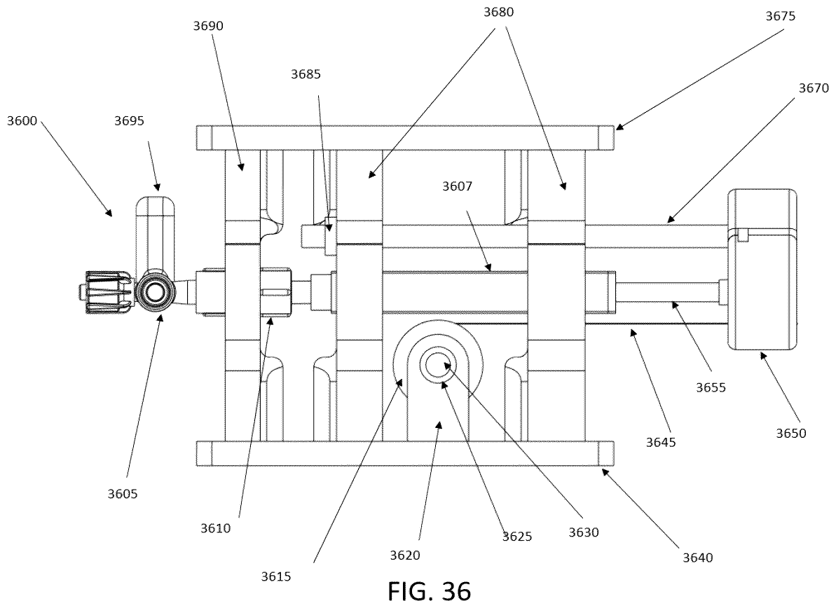

FIG. 36 shows a side view of a fluid dispersion device 3600 according to some embodiments. Shown is the valve 3605, the valve lever 3695 for opening the valve, the syringe adapter 3610, the spring spool 3615, the spring spool support 3620, the spring spool axle bushing 3625, the spring spool axel 3630, the base plate 3640, the spring 3645, the syringe plunger 3655, the syringe plunger attachment plate 3650, the guide rod 3670, the upper plate 3675, the syringe supports 3680, the guide rod brushing 3685, and the syringe adapter 3690. In some embodiments, the spring 3645 may be a constant force spring and may apply a force of the same or approximately the same magnitude along the length for of the displacement. The spring may pull the syringe plunger attachment plate 3650 towards the front end of the device and push the plungers into the syringes pushing out the fluid contained therein at a consistent rate.

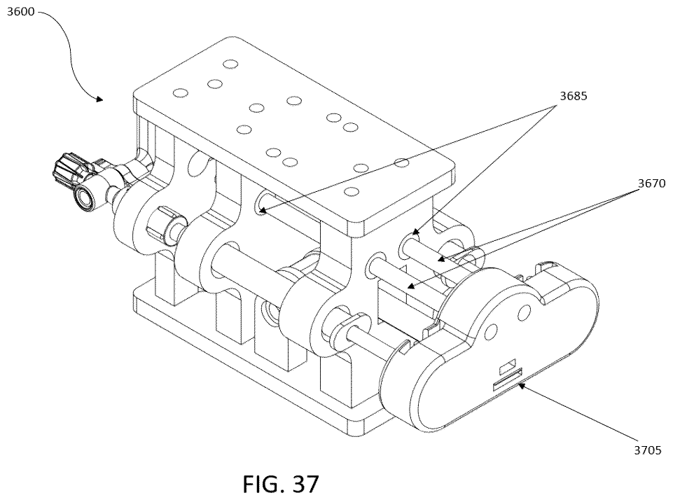

FIG. 37 shows a front perspective view of a fluid dispersion device according to some embodiments. Shown are the guide rods 3670, the spring attachment slot 3705, the guide rod brushing 3685, the syringe plunger 3655, and the syringe plunger attachment plate 3650.

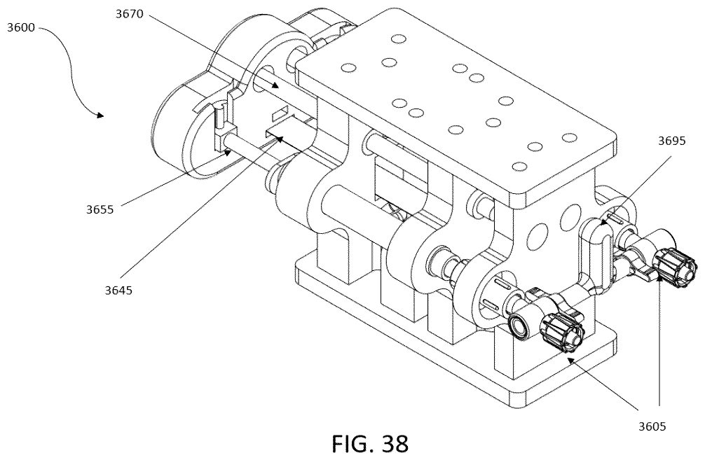

FIG. 38 shows a rear perspective view of a fluid dispersion device according to some embodiments. Shown are the guide rods 3670, the plungers 3655, the spring 3645, the valves 3605, and the valve trigger 3695.

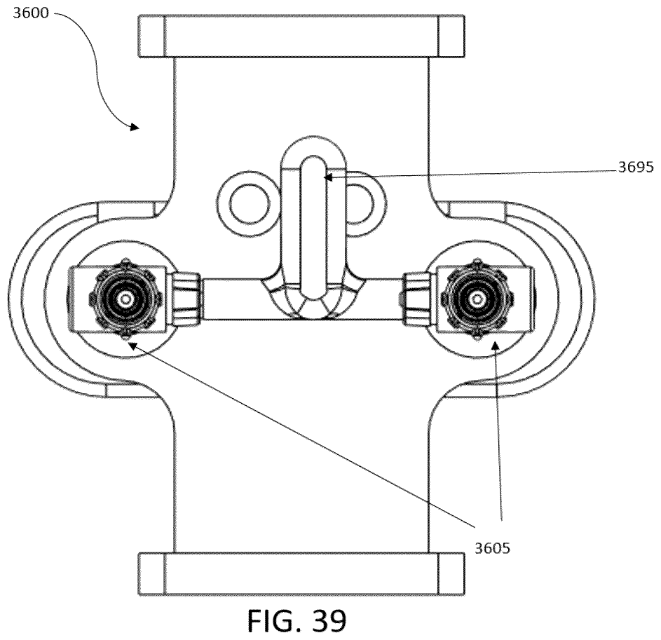

FIG. 39 shows front view of a fluid dispersion device according to some embodiments. Shown are the valves 3605, and the valve trigger 3695.

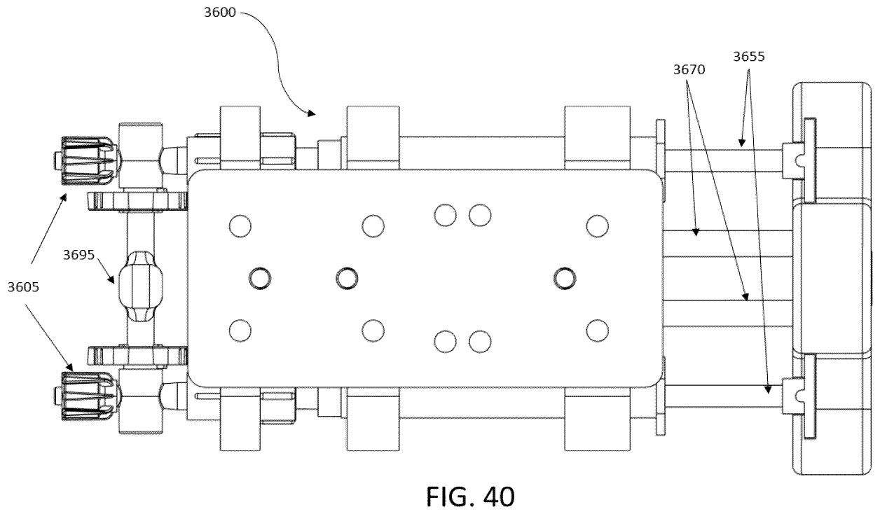

FIG. 40 shows a top view of a fluid dispersion device according to some embodiments. Shown are the guide rods 3670, the plungers 3655, the valves 3605 and the valve triggers 3695.

In some embodiments, the device may be connected to a trigger attached to the upper plate which releases the constant force spring and pushes fluid out from the syringes. In some embodiments, there may be a hose attached to the valves which connects to a dispersion nozzle and a compressed air line, that aerosolizes the fluid as it is dispersed from the nozzle. In some embodiments, the resulting stiffness of an ECM gel dispersed from the device may vary depending on the nozzle geometry, the nozzle offset, and the air pressure.

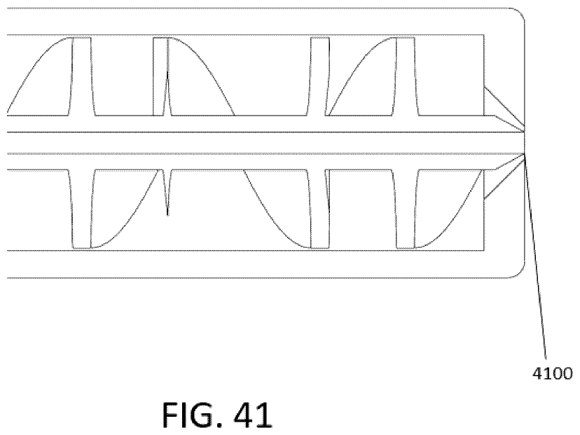

FIG. 41 shows a cross sectional view of a nozzle with a 0 offset 4100 of some embodiments. In some embodiments, a 0 offset may mean that the nozzle may be flush with the point of dispersion of the fluid. In some embodiments, a 0 offset may result in a gel or ECM with increased stiffness and higher storage modus.

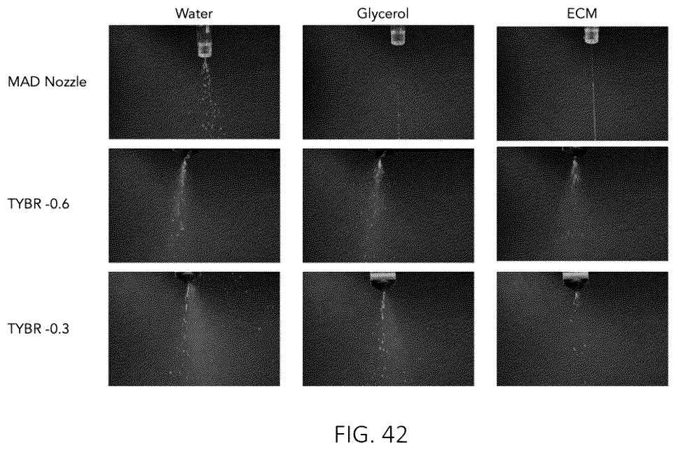

FIG. 42 shows the resulting aerosolization of water, glycerol, and ECM fluid through varying nozzles of some embodiments. The left column shows water dispersed through a MAD nozzle, a TYBR 0.6 nozzle, and a TYBR 0.3 nozzle; the center column shows glycerol dispersed through a MAD nozzle, a TYBR 0.6 nozzle, and a TYBR 0.3 nozzle; and the right column shown ECM dispersed through a MAD nozzle, a TYBR 0.6 nozzle, and a TYBR 0.3 nozzle. As can be observed, the MAD nozzle had the worst dispersion of fluid in all cases, and failed to aerosolize any of the fluids. The TYBR 0.6 nozzle provided the second best dispersion of fluid and obtained a moderate aerosolization of fluid in each case, including the high viscosity glycerol and ECM fluids. The TYBR 0.6 nozzle obtained the best fluid dispersion of the three nozzles and obtained an aerosolization of all fluids dispersed through the nozzle, including the high viscosity glycerol and ECM fluids.

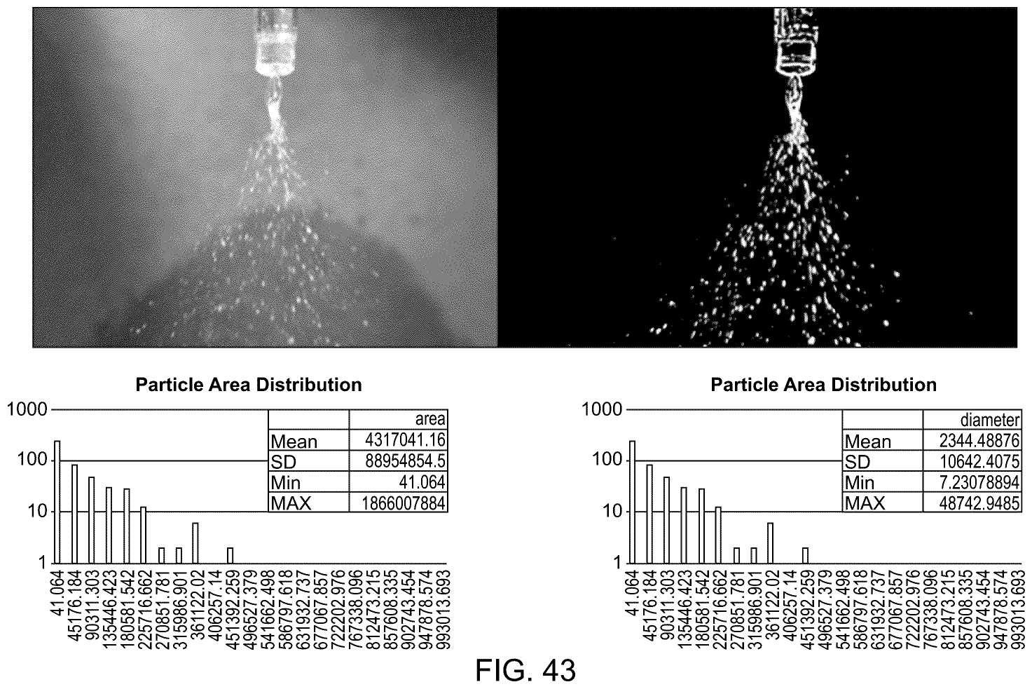

FIG. 43 shows a fluid dispersion of water through a MAD nozzle and graphs of the particle area and particle diameter distribution according to some embodiments. As can be observed from the images, the MAD nozzle failed to aerosolize the fluid produced a fluid dispersion with an average droplet diameter of 2,344 micrometers, and an average particle area distribution of 4.3 million square micrometers.

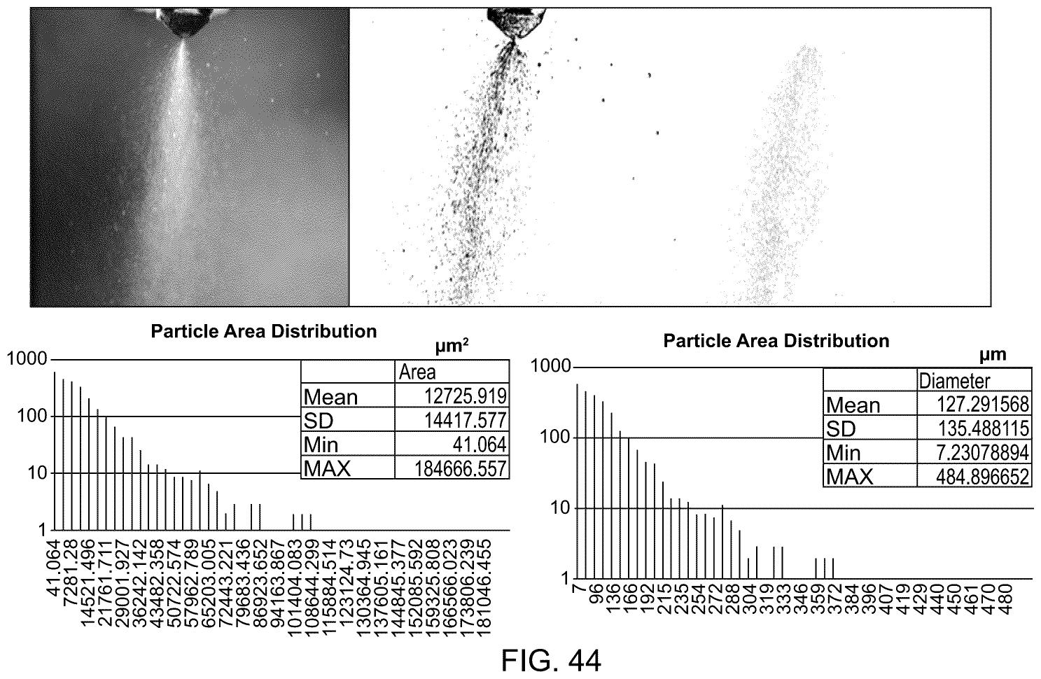

FIG. 44 shows a fluid dispersion of water through a TYBR 0.6 nozzle and graphs of the particle area and particle diameter distribution according to some embodiments. As can be observed by the images, the TYBR 0.6 nozzle obtained aerosolization of the fluid and produced a fine mist with an average particle diameter of about 128 micrometers, and a average particle area distribution of 12.7 thousand square micrometers. It can further be observed from the particle diameter distribution graph that the significant majority of the particles had particle diameter size between 7 and 136 micrometers, indicating a near complete aerosolization of the fluid.

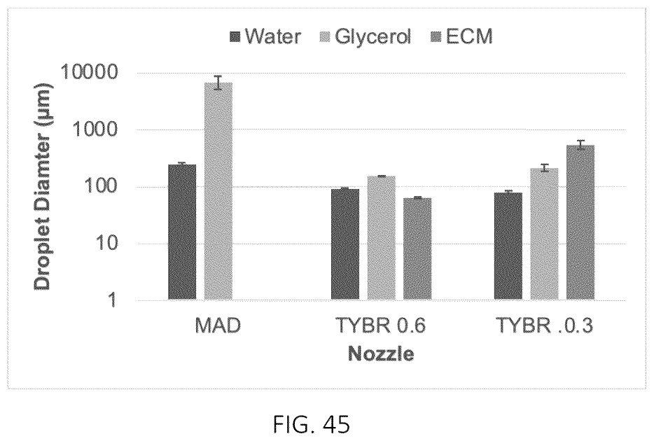

FIG. 45 shows a graph further illustrating the particle diameter distribution according to some embodiments resulting from distribution of water, glycerol, and an ECM hydrogel from a MAD nozzle, an TYBR 0.6 nozzle, and a TYBR 0.3 nozzle. It is shown that when dispersing water or glycerol through the MAD nozzle that a water droplet diameter exceeding 100 um was obtained, a droplet diameter of nearly 10,000 um was achieved for glycerol, and that the ECM hydrogel failed to disperse into droplets whatsoever and was only dispersed as a stream. It is shown that when dispersing water, glycerol, or the ECM hydrogel through the TYBR 0.6 nozzle that a water droplet diameter below 100 um was obtained, a glycerol droplet diameter of nearly 100 um was achieved for glycerol, and that the ECM hydrogel was successfully aerosolized with droplet diameter of well below 100 um. It is shown that when dispersing water, glycerol, or the ECM hydrogel through the TYBR 0.3 nozzle that a water droplet diameter below 100 um was obtained, a glycerol droplet diameter of exceeding 100 um was achieved for glycerol, and that the ECM hydrogel was aerosolized to a reduced degree when compared to the TYBR 0.6 nozzle with droplet diameter approaching 1000 um. This data is further summarized below in Table 1.

TABLE 1
Droplet and Particle Diameter

| Fluid | Nozzle | Diameter (um) | SEM |
|---|---|---|---|
| Water | MAD | 248 | 17 |
| | TYBR 0.6 | 91 | 3 |
| | TYBR 0.3 | 79 | 3 |
| ECM | MAD | N/A | N/A |
| | TYBR 0.6 | 63 | 2 |
| | TYBR 0.3 | 541 | 92 |
| Glycerol | MAD | 6815 | 1797 |
| | TYBR 0.6 | 152 | 4 |
| | TYBR 0.3 | 213 | 31 |

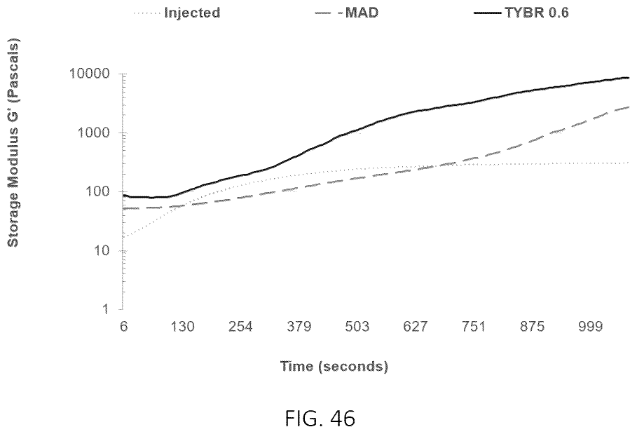

FIG. 46 shows a graph of the storage modulus of the ECM hydrogel vs time when dispersed through a MAD nozzle, a TYBR 0.6 nozzle, and when injected. It can be observed that the TYBR 0.6 produced cross-linked hydrogel with the highest storage modulus, exceeding 1000 Pa at 500 seconds, and approaching 10,000 Pa at 1000 seconds. The MAD nozzle produced a cross-linked hydrogel with storage modulus of about 200 Pa at 500 seconds, and less than 1,200 Pa at 1000 seconds. The injected ECM hydrogel achieved a maximum storage modulus of less than 1,200 Pa at 1000 seconds.

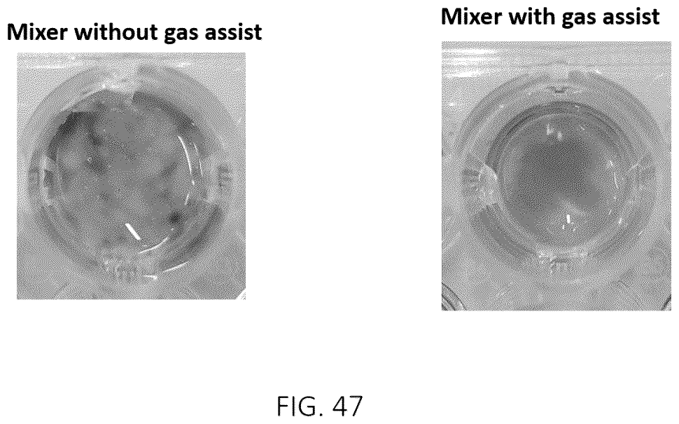

FIG. 47 shows illustrative images of the ECM hydrogel dispersed from a nozzle with gas assist and a nozzle without gas assist. In the image without gas assist it can be observed that there is reduced mixing in the sample dispersed from a nozzle without gas assist and air bubbles trapped in the hydrogel matrix. In the image with the gas assist it can be observed that there is now nearly homogenous mixing of the ECM hydrogel in the sample, and reduction in the amount of air bubbles trapped in the hydrogel matrix.

### EXAMPLES

### Example 1: Determination of Nozzle Design on Droplet Size and Aerosolization Length

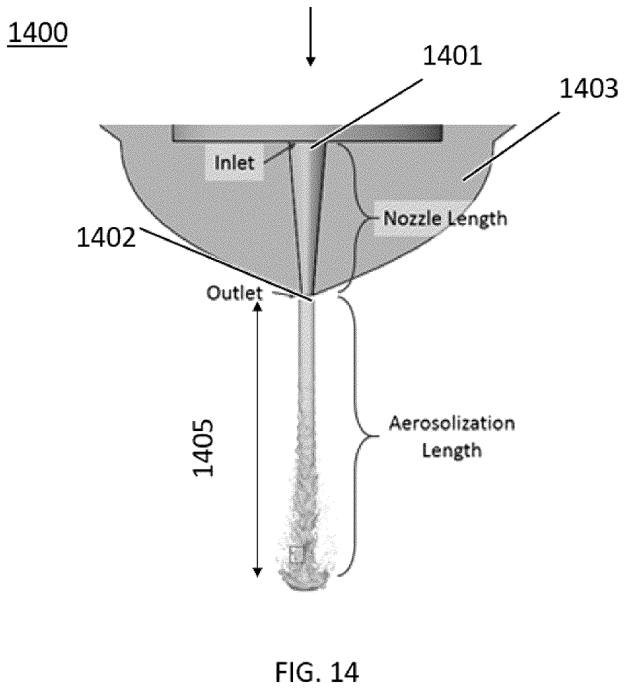

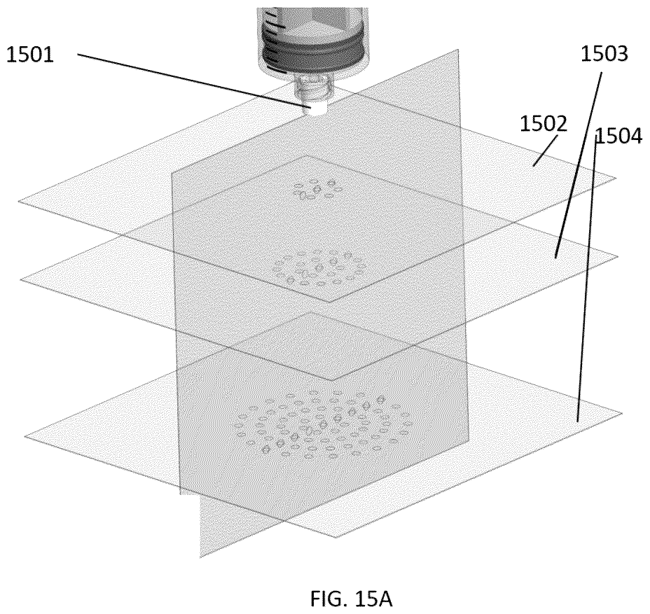

One of the primary functions of the nozzle is to aerosolize the anti-adhesive mixture. Performance of the nozzle may affect application and usability in a surgical setting. To assess the performance of the nozzle, droplet size, spray pattern, aerosolization length, and material characteristics of the resultant gel were measured. An aerosolization length can be defined as a distance from a nozzle outlet where droplets are formed from a jet of fluid dispersed from the nozzle. Droplet size can affect surface coverage achieved by dispersing a fluid from a spray device. Other fluid mechanics or kinetics of the droplets or the jet of fluid dispersed from a spray device can be affected by the spray device features (e.g., geometry of the nozzle). Other features may comprise, a velocity of the droplets or the jet of fluid, gel formation from the multicomponent fluid, or an aerosolization length. Aerosolization length may affect usability in a confined space (e.g., small cavity). For example, in a laparoscopic surgery, an abdominopelvic cavity may be insufflated with carbon dioxide to create a workspace; the space is constrained by the distance between the peritoneal wall and the underlying tissue. This space could range from about 2 cm to about 10 cm depending on patient and insufflation pressure, which may be about 10 mmHg to about 20 mmHg. An aerosolization distance of at most about 2 cm to about 10 cm may be required to ensure a uniform coverage of the fluid being dispersed form the spray device on the tissue. FIG. 14 shows a schematic depiction of a fluid being dispersed from a nozzle 1400, having an aerosolization length of 1405 (reference: (Shinjo2010—Simulation of liquid jet primary breakup_Dynamics of ligament and droplet formation doi: 10.1053/j.gastro.2016.04.002).

The unidirectional arrow shows a direction of fluids delivered to the inlet from the mixer chamber and/or dispersant passageway. The nozzle may comprise a nozzle inlet 1401, a nozzle outlet 1402, and a nozzle body 1403. The nozzle body 1403 may comprise round corners. In some cases, the nozzle body comprises square corners.

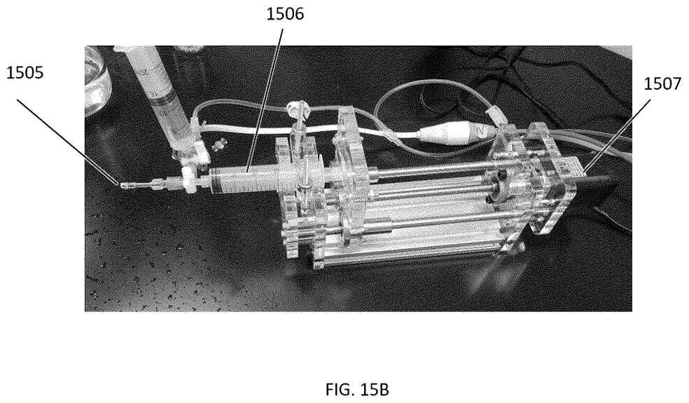

FIG. 15A shows a schematic of an experiment design to measure an aerosolization length of a fluid dispersed from a nozzle. To investigate a performance of a nozzle 1501 to aerosolize a fluid (e.g., an anti-adhesive mixture), the fluid was sprayed out of the nozzle. The fluid may be delivered to the nozzle by a pressure inducing device (e.g., a spraying device, a syringe). In the following examples the fluid was delivered into a nozzle using a syringe. A flow rate of the fluid may be controlled by using a syringe pump that may be set to generate a predefined flow rate (e.g., equivalent to a flow rate of a spray device described herein). The dispersion of the fluid as a jet, stream, or droplets were observed in multiple horizontal planes of known distance from the exit of the nozzle 1502, 1503, or 1504. Data generated from the observation was used to describe the shape of the dispersed fluid (e.g., a jet, a stream, or droplets). Size of the droplets (e.g., a droplet diameter) at the horizontal plane were also measured from the data obtained from the observation. at each location. FIG. 15B shows an example of a syringe pump device used in this experiment. A fluid may be delivered to a nozzle 1505 (Mad Nasal™) using a syringe 1506 and be injected from the nozzle. The rate of injection (e.g., fluid pressure) may be controlled using an electronic syringe pump 1507.

### Example 2: Aerosolization Capabilities of Various Nozzle Geometries

The aerosolization length and droplet size may in part depend on the shape and length of the inlet of the nozzle, nozzle length, the outlet of the nozzle. In order to investigate or optimize the nozzle, different nozzles with various inlet diameters, outlet diameters, or lengths were tested using the method described hereinbefore and depicted in FIG. 15A.

Eight nozzle geometries were compared with distinct inlet diameters, outlet diameters, and nozzle lengths as described in Table 2. All the nozzles tested in this example were produced using additive manufacturing (e.g., 3D printing) and comprised a circular inlet and a circular outlet. For each nozzle geometry, a Luer lock connector syringe filled with either water or vegetable oil was inserted to determine the impact of the fluid of different viscosities on aerosolization and droplet formation, and pressure was applied. The resulting jet was visually assessed for presence of aerosolization, as described in example 1. If any aerosolization was present, further quantitative evaluation was undertaken. A computer-implemented analysis can be used to analyze the data collected with respect to droplet pattern and coverage.

TABLE 2
Geometries and specifications of eight nozzles to test aerosolization and droplet formation

| | | | | | | | | |
|---|---|---|---|---|---|---|---|---|
| Inlet Shape | Circular | Circular | Circular | Circular | Circular | Circular | Circular | Circular |
| Outlet Shape | Circular | Circular | Circular | Circular | Circular | Circular | Circular | Circular |
| Inlet Diameter | 0.5 mm | 0.5 mm | 0.5 mm | 1.0 mm | 1.0 mm | 0.15 mm | 0.25 mm | 0.25 mm |
| Outlet Diameter | 0.15 mm | 0.25 mm | 0.25 mm | 0.25 mm | 0.25 mm | 1.0 mm | 1.0 mm | 1.0 mm |
| Nozzle Length | 2.0 mm | 2.0 mm | 0.5 mm | 2.0 mm | 0.5 mm | 0.5 mm | 2.0 mm | 0.5 mm |

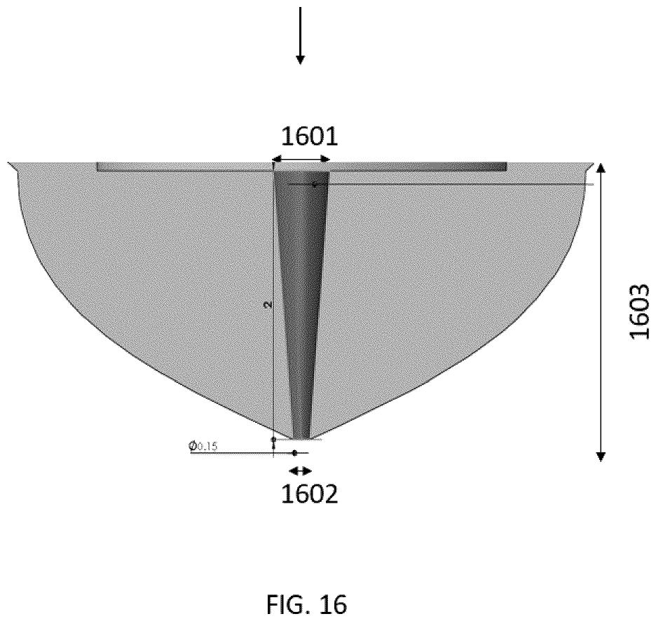

FIG. 16 shows a nozzle (e.g., N01) configured to have an inlet diameter 1601 of about 0.5 millimeter (mm), an outlet diameter 1602 of about 0.15 mm, and a length 1603 of about 2.0 mm. The unidirectional arrow shows a direction of fluids delivered to the inlet 1601 from the mixer chamber and/or dispersant passageway.

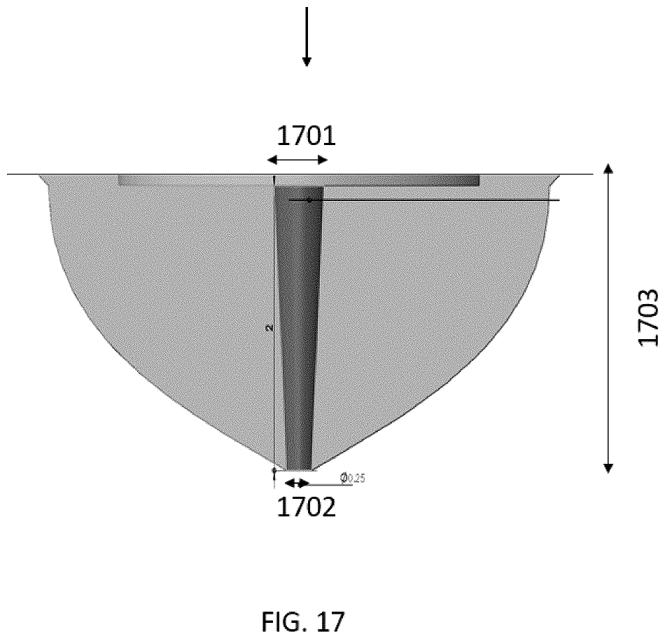

FIG. 17 shows a nozzle (e.g., N03) configured to have an inlet diameter 1701 of about 0.5 millimeter (mm), an outlet diameter 1702 of about 0.25 mm, and a length 1703 of about 2.0 mm. The vegetable oil and water formed a jet of fluid when injected through the nozzle. The unidirectional arrow shows a direction of fluids delivered to the inlet 1701 from the mixer chamber and/or dispersant passageway.

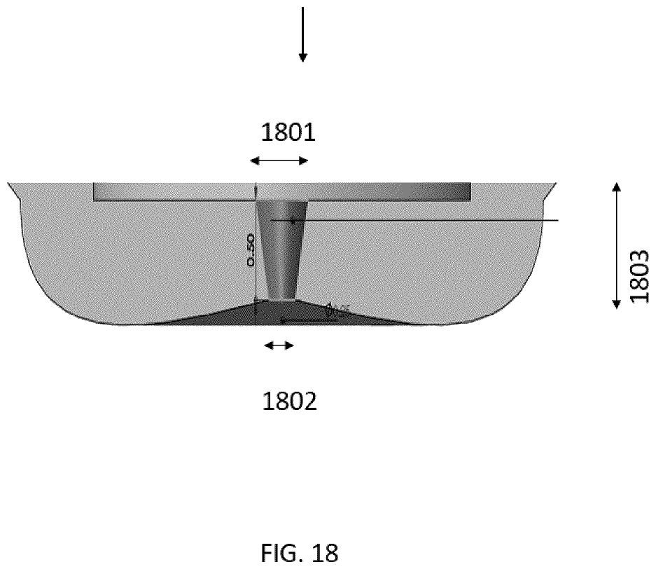

FIG. 18 shows a nozzle (e.g., N04) configured to have an inlet diameter 1801 of about 0.5 millimeter (mm), an outlet diameter 1802 of about 0.25 mm, and a length 1803 of about 0.5 mm. The vegetable oil and water formed a jet of fluid when injected through the nozzle. The unidirectional arrow shows a direction of fluids delivered to the inlet 1801 from the mixer chamber and/or dispersant passageway.

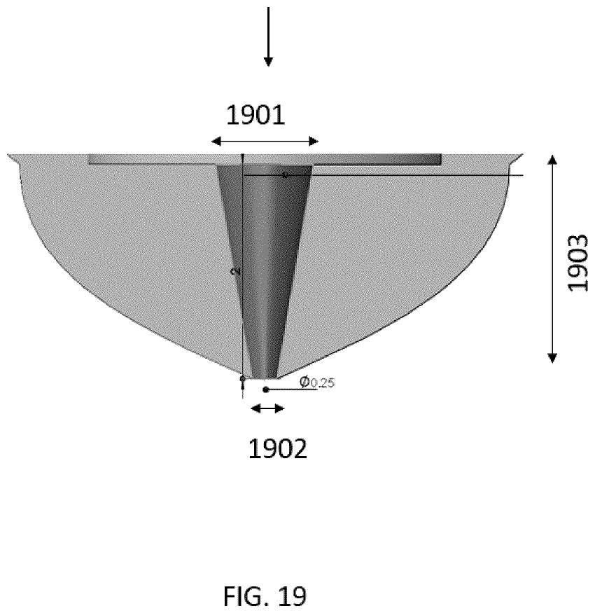

FIG. 19 shows a nozzle (e.g., N07) configured to have an inlet diameter 1901 of about 1.0 millimeter (mm), an outlet diameter 1902 of about 0.25 mm, and a length 1903 of about 2.0 mm. Water was aerosolized, but the vegetable oil did not aerosolize. The unidirectional arrow shows a direction of fluids delivered to the inlet 1901 from the mixer chamber and/or dispersant passageway.

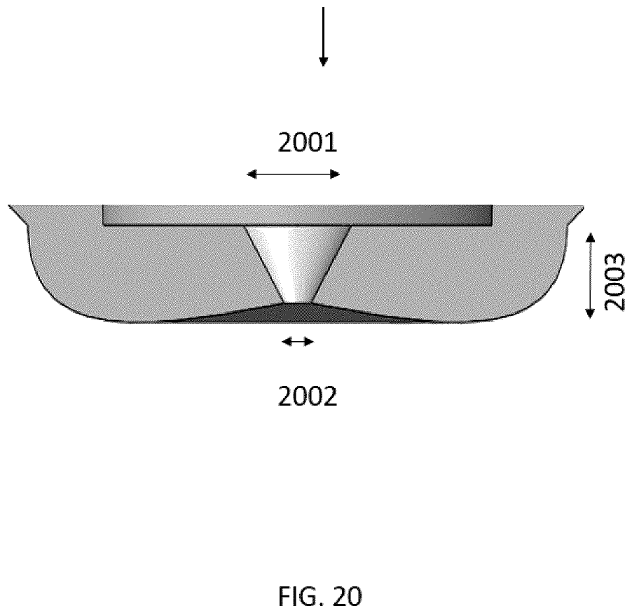

FIG. 20 shows a nozzle (e.g., N08) configured to have an inlet diameter 2001 of about 1.0 millimeter (mm), an outlet diameter 2002 of about 0.25 mm, and a length 2003 of about 0.5 mm. A jet of fluid was formed for both the vegetable oil as well as the water. The unidirectional arrow shows a direction of fluids delivered to the inlet 2001 from the mixer chamber and/or dispersant passageway.

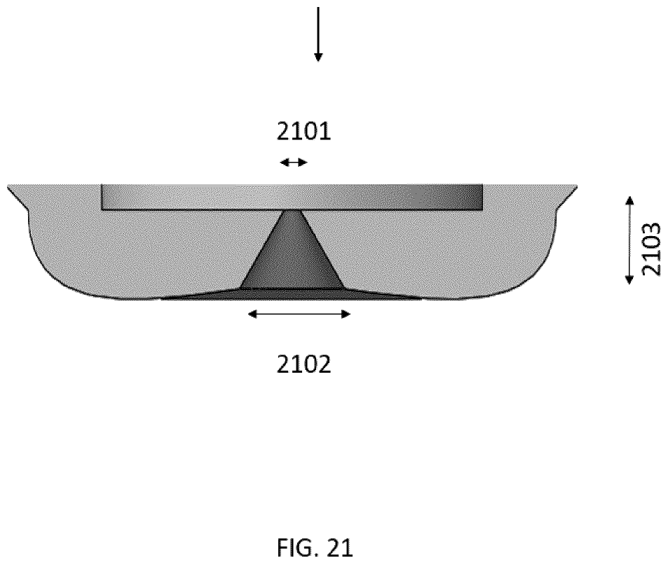

FIG. 21 shows a nozzle (e.g., N12) configured to have an inlet diameter 2101 of about 0.15 millimeter (mm), an outlet diameter 2102 of about 1.0 mm, and a length 2103 of about 0.5 mm. A fluid outlet was not observed. It was suspected that the 3D printed nozzle was rendered incapable of releasing a fluid from the outlet. The unidirectional arrow shows a direction of fluids delivered to the inlet 2101 from the mixer chamber and/or dispersant passageway.

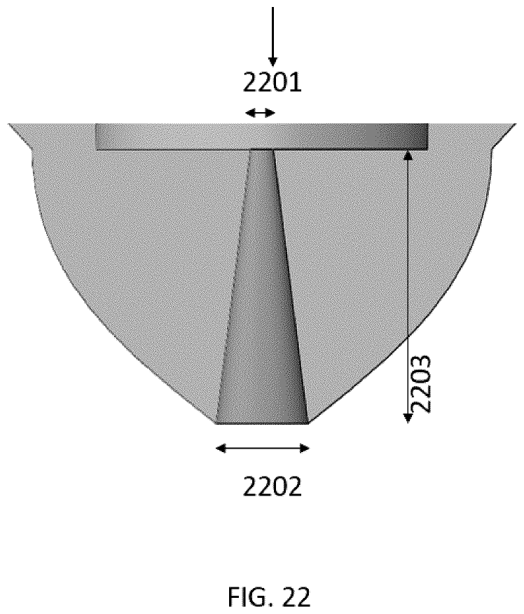

FIG. 22 shows a nozzle (e.g., N15) configured to have an inlet diameter 2201 of about 0.25 millimeter (mm), an outlet diameter 2202 of about 1.0 mm, and a length 2203 of about 2.0 mm. Water was aerosolized, but the vegetable oil did not aerosolize. The unidirectional arrow shows a direction of fluids delivered to the inlet 2201 from the mixer chamber and/or dispersant passageway.

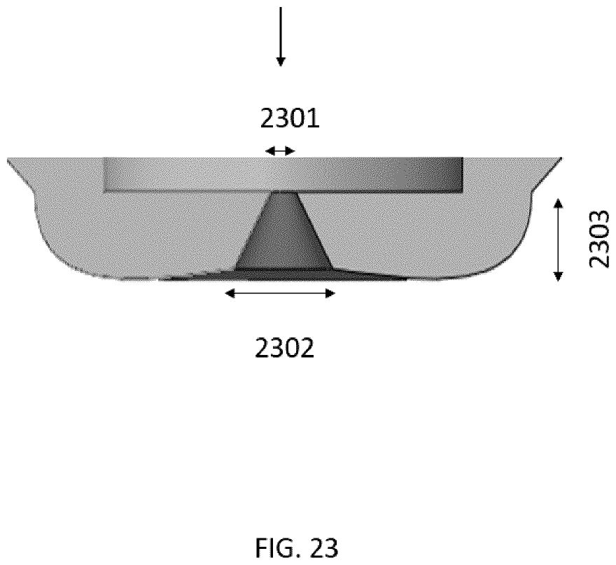

FIG. 23 shows a nozzle (e.g., N16) configured to have an inlet diameter 2301 of about 0.25 millimeter (mm), an outlet diameter 2302 of about 1.0 mm, and a length 2303 of about 0.5 mm. Water was aerosolized, but the vegetable oil did not aerosolize. The unidirectional arrow shows a direction of fluids delivered to the inlet 2301 from the mixer chamber and/or dispersant passageway.

### Example 3: Aerosolization Capabilities of Various Nozzle Geometries

In order to investigate an effect of a viscosity of a fluid being sprayed using a nozzle, fluids with different viscosities were injected into a conical nozzle that is commercially available (MAD Nasal™) (similar to nozzle 1400 in FIG. 14) at a pressure of about 700 MPa (or approximately 100 PSI). Aerosolization of the sprayed fluids was observed. FIG. 24A-24C show exemplary images of aerosolization of three fluids with different viscosities, all using the commercially available nozzle (MAD Nasal™). FIG. 24A shows an exemplary image of an aerosolization of a fluid with a viscosity of about 0.9 centipoise (cP). The fluid was aerosolized 2402 after being sprayed out of the nozzle 2401. FIG. 24B shows an exemplary image of a fluid with a viscosity of about 13.8 cP (e.g., a solution of 75% glycerin) being sprayed out of the nozzle 2403. A jet of fluid was formed 2404. FIG. 24C shows an exemplary image of a fluid with a viscosity of about 1,115 cP (e.g., a solution of 100% glycerin) being sprayed out of the nozzle 2405. The fluid with the high viscosity formed large droplets 2406. The highly viscous fluid did not generate aerosols or a jet using the nozzle as described here.

### Example 4: Method of Operation

During a surgical procedure in the abdomen or pelvis, access sites are created, either by minimally invasive techniques including laparoscopic and robotic approaches or by traditional opened surgeries such as laparotomies. The surgeon may insert appropriate instruments to perform the procedure. At the conclusion of the procedure all surgical tools and instruments can be withdrawn. At this point, a nozzle end of a spray device, as described herein, can be inserted through the access site. The nozzle end of the spray device may be guided to the site of the procedure by use of a steering mechanism, as described herein. Upon pressing the button of a controller to release fluids from the fluid containers, the dispenser can depress the plungers such that their contents move through the multi-lumen tube where they enter the mixer. In the mixer, the fluid components from the fluid containers are subjected to mixing to form a multi-component fluid. At the distal end of the mixer, a multi-component fluid exits through the nozzle with a gas dispersant (e.g., compressed air) to form small droplets (e.g., aerosolized fluid, atomized fluid). The multi-component fluid can begin to form a gel upon physical contact with the tissue by absorbing heat from the tissue or by other means (i.e. light). The user (e.g., a surgeon, an operator, etc.) may spray the surface of the organ and abdominal wall until they are coated with a coat of the multi-component fluid. The spray device can be withdrawn, and surgical access sites are closed.

### Example 5: Example of Comparison of a Commercially Available Nozzle and Custom Nozzle

In this experiment, a commercially available atomization device, MAD Nasal™ (Teleflex Inc., Wayne, PA, USA) was compared to a custom nozzle prepared for delivering a multi-component fluid. The multi-component fluid comprised of ECM hydrogel (e.g., ECM and a buffer) in its liquid phase for the testing. The liquid is highly thixotropic, with a viscosity ranging from 1,000,000 cP at low shear to 10 cP at high shear. In particular, a shear-thinning behavior was observed, wherein the viscosity markedly dropped with increasing shear rates. Moreover, the pseudoplastic behavior of the ECM hydrogel is demonstrated by the characteristic shape of the shear stress curve, as shown in FIG. 25. The MAD Nasal™ device uses the kinetic energy of the fluid to create small droplets. As the fluid is accelerated through a small orifice, the flow becomes predictably unstable and aerosolization occurs. Though this device produces droplets of a predictable size, it is only designed for use with low viscosity fluids and fails to produce small droplet when used with viscous fluids.

The nozzle disclosed herein does not rely on the kinetic energy of the fluid itself to aerosolize; rather it employs a gas dispersant to break the fluid into droplets. FIG. 27 shows a cross-section of the nozzle. The nozzle has an outlet diameter 2702 of about 1.6 millimeter (mm). The gas dispersant passage way 2705 has a dispersant outlet diameter (e.g., orifice diameter) 2701 of about 0.6 mm. The distance between the dispersant outlet and the nozzle outlet (or offset) 2703 is about 0.25 mm. A coaxial, multi-lumen nozzle dispenses ECM hydrogel through the outer channel (2706) and compressed gas through the inner channel (e.g., 2705) to disperse the ECM into droplets.

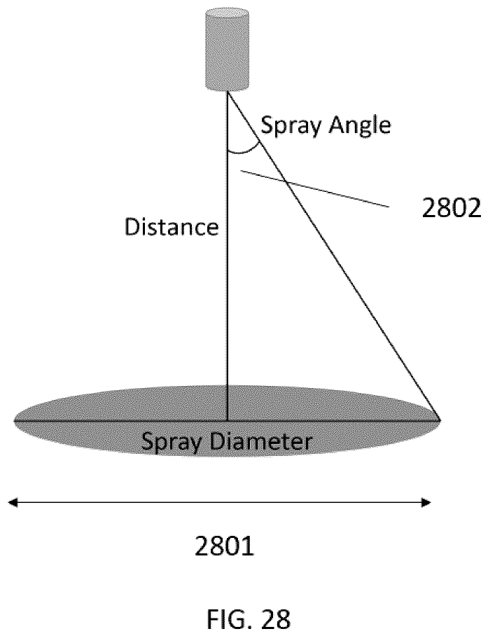

The nozzles were evaluated by a spray pattern and stiffness of the resulting gel. FIG. 28 shows the spray pattern comprising a spray diameter 2801 and spray angle 2802. The spray pattern was measured by ejecting 0.5 mL of ECM at 15 mL/min onto a target from distances ranging from 2 to 10 cm; then the diameter of the covered area was measured. Since both distance and diameter are known, the spray angle can be calculated by basic trigonometry. Each nozzle was sprayed at each measurement plane four times so an average and standard deviation could be calculated. The ECM was also sprayed onto a parallel plate rheometer (MCR 302, Anton Paar GmbH, Graz, Austria). The rheometer maintained 37° C., so the liquid ECM hydrogel transitioned into a solid gel. The stiffness of each resulting gel was measured.

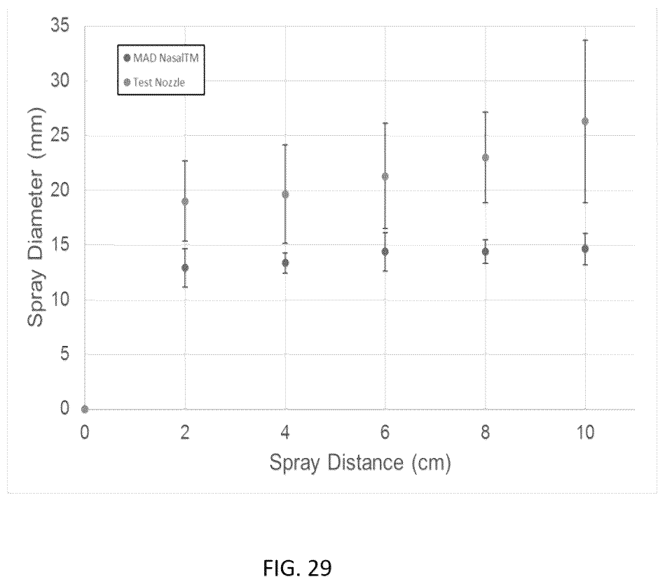

When spraying liquid ECM hydrogel, the spray angle of the MAD Nasal™ nozzle was 8.7° (though it ejected primarily as a jet rather than a spray), and the spray angle of the proposed nozzle was 13.0°. The spray diameter over spray distance is shown in FIG. 29.

It is noteworthy that the calculated average spray angle underestimates the spray diameter near the nozzle and overestimates the spray diameter expected far from the nozzle. This suggests that the spray pattern is not conical; rather it expands rapidly out of the nozzle but does not continue to expand as it travels away. This is true of both the gas-assist and non-gas-assist nozzle. It is possible that the ECM gel had enough momentum upon impact when sprayed from a short distance that it continued to spread; this will be assessed further in future studies.

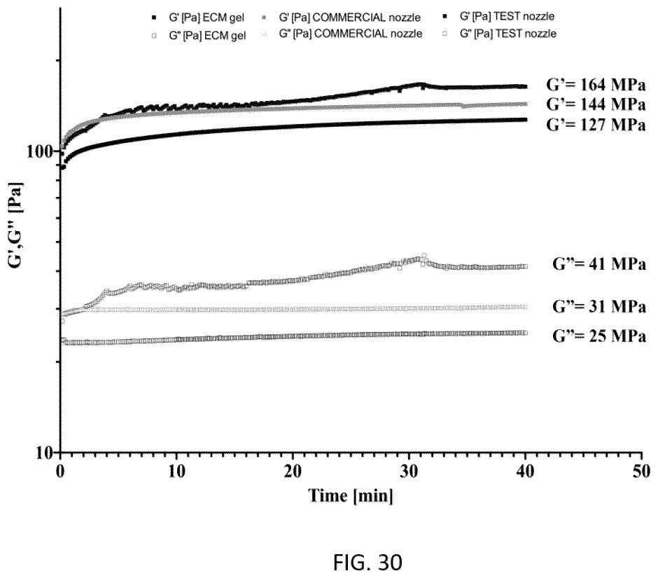

The stiffnesses of the gels resulting from injected, sprayed from the MAD Nasal™ nozzle, and sprayed from the test nozzle are shown in FIG. 30. Hydrogel stiffness was determined through the evaluation of storage and loss moduli, respectively G′ and G″. In the solid gel phase (37° C.), G′ defines the overall behavior of the hydrogel. The injected ECM formed the weakest gel, with G′ of 127 MPa. However, when ECM was sprayed with the gas assisted test nozzle the stiffness increased up to G′=164 MPa. With MAD Nasal' sprayed ECM gel, G′ plateaued at 144 MPa. The results showed that although the MAD Nasal™ device reliably aerosolizes low viscosity fluids, it does not easily aerosolize high viscosity fluids like liquid ECM. Based on spray pattern analysis and rheologic evaluation, it is concluded that the proposed nozzle with gas-assisted aerosolization is better for use with viscous fluid.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 6: Orthopedic Surgery

In this example, the fluid dispersion device and the ECM fluid of one or more embodiments are used to produce an adhesion barrier and improve patient outcomes in an orthopedic surgery.

A surgeon is performing a full reconstruction of an extensor pollicis longus tendon of the hand which was fully severed. The surgeon begins the repair as a traditional surgery by dissection of the sheath to expose the tendon, and suturing of the two severed ends of the tendon back together. Following repair of the tendon, but prior to closing the surgical site; the surgeon applies an adhesion barrier comprising ECM fluid using the fluid dispersion device of present embodiments.

The device comprises a TYBR 0.6 nozzle, ECM fluid contained within a pair of dual syringes connected to a constant force spring. The TYBR 0.6 nozzle is connected to a compressed CO2 source which disperses compressed CO2 from the center of the nozzle, and aerosolizes the fluid to a high stiffness ECM hydrogel as it is dispersed from the nozzle. The compressed CO2 is pressurized to 10 psig.

Prior to closing the wound and following tendon repair, the surgeon places the nozzle approximately three inches away from the surgical site and triggers the valve, resulting in aerosolization of the ECM fluid as it is dispensed from the nozzle and dispersed into a fine mist with an average particle diameter between 100 micrometers to 250 micrometers. The resulting ECM gel has a storage modulus between 2500 and 3000 Pa and evenly coats the surface of the repaired tendon. The wound is then closed and any remaining CO2 is dissolved in the blood and safely eliminated through normal physiological processes. The storage modulus of the ECM gel is less than the elastic modulus of the tendon and the surrounding tissue, and does not impede the biomechanics of tendon gliding and tendon pulling.

Following the surgery and application of the adhesion barrier, the injured tendon is not in direct contact with the injured tendon sheath tissue, and glides in the tendon sheath with minimal friction when compared to an injured tendon sliding in the tendon sheath in the absence of an ECM barrier of present embodiments. The presence of the adhesion barrier between the injured tendon and the injured tendon sheath tissue prevents the formation of significant scar tissue, and allows the tendon to heal with proper tendon gliding, proper joint mechanics, and a nearly full range of motion. This improved subject outcome is obtained with reduced pain, fewer courses of physical therapy, and less recovery time relative to a same tendon repair accomplished in the absence of application of the ECM hydrogel adhesion barrier.

The patient outcome is superior to application of current orthopedic adhesion barriers (VersaWrap, TenoMend, TenoGlide) which are dehydrated sheets which are brittle when dry but begin to disintegrate when wet, and fail to provide an even adhesion barrier with a high storage modulus. Further, the adhesion barrier comprising ECM fluid using the fluid dispersion device of present embodiment permits an even coating of irregular and confined anatomies such as between the flexor tendon and the flexor tendon sheath in an easy, quick, and simple manner ideal for application in a surgical setting.

### Example 7: Abdominal Surgery—Colorectal Resection & Ostomy Creation

In this example, the fluid dispersion device and ECM fluid of one or more embodiments are used to produce an adhesion barrier, prevent the formation of adhesions along the small intestine, large intestine, and to improve patient outcomes in a colorectal resection, and an ileostomy creation procedure.

A subject has suffered a perforation of the sigmoid colon approximately 8 inches above the rectum. A surgeon is performing colorectal resection of a subject's sigmoid colon a result of the colorectal perforation, and diverting the subjects bowel just below the small intestine as part of an ileostomy creation. The surgeon is performing the surgery using a laparoscopic surgery method to minimize the trauma to the subject resulting from the surgery.

The device comprises a 0.6 mm laparoscopic nozzle, ECM fluid contained within a syringe connected to a constant force spring and dispersed through an annular area of the nozzle. The 0.6 mm laparoscopic nozzle is connected to a compressed CO2 source which disperses compressed CO2 from the center of the nozzle, and aerosolizes the fluid to a high stiffness ECM hydrogel as it is dispersed from the nozzle. The compressed CO2 is pressurized to 10 psig.

The colorectal recession is performed as in known within the art, following suture of the two sections of healthy colon tissue to one another, the surgeon places a laparoscopic nozzle approximately 0.1-2.0 inches away from the suture site and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 180 degrees in each direction relative to a starting point to evenly coat the entire surface of the repaired bowel tissue. The resulting ECM gel has a storage modulus between 2500 and 3000 Pa and evenly coats the surface of the repaired intestinal tissue. The laparoscopic incisions are then closed, and any remaining CO2 is dissolved in the blood and safely eliminated through normal physiological processes. The storage modulus of the ECM gel is less than the elastic modulus of the sigmoid colon and the surrounding tissue (e.g., 20-40 kPa), and does not impede the biomechanics of bowel function, including bowel movements. The resulting ECM barrier formed over the repaired bowel tissue operates to permit gliding of the abdominal wall over the sigmoid colon tissue, and permits gliding of the large intestine tissue over other sections of sigmoid colon tissue it is in contact; and prevents the formation of post-surgical adhesions. Also as a result of the application of the application of the adhesion barrier, there is a 50% reduction in the formation of scar tissue resulting from the colorectal rescission.

In parallel with the colorectal resection, an ileostomy creation procedure is performed as is known within the art. In this example, an ileostomy procedure is performed by bringing the incised section of the small intestine, the stoma, to the wall abdominal wall and suturing the small intestine in place. At this time, an adhesion barrier is applied to the surface of the incised small intestine tissue, along the sutured section, along the section of tissue extending outward from the body, and along the surface of the tissue remaining within the body, effectively coating the entire section of incised tissue with the ECM barrier.

The surgeon places a dispensing nozzle approximately 0.5-3.0 inches away from the suture site and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 180 degrees in each direction relative to a starting point to evenly coat the entire surface of the incised and sutured small intestinal tissue. The resulting ECM gel has a storage modulus between 2500 and 3000 Pa and evenly coats the surface of the repaired intestinal tissue. The incisions are then closed with the stoma extending outward from the subject, and an ileostomy bag placed over the stoma. Any remaining CO2 is dissolved in the blood and safely eliminated through normal physiological processes. The storage modulus of the ECM gel is less than the elastic modulus of the large intestine and the surrounding tissue, and does not impede the biomechanics of digestion, or bowel movement.

In the months following the surgery, once the section of the large intestine which has been repaired has begun to heal, when inflammation has receded, and once any resulting infections have been controlled or eradicated, the subject reports for an ileostomy repair procedure.

The ileostomy repair procedure is performed as is known within the art. When the surgeon begins to lyse adhesions, approximately 80% fewer adhesions are noted to have been formed as a result of ileostomy creation, and the adhesions which have been formed are 50% smaller in size relative the size of intestinal adhesions traditionally resulting from such procedures. The ileostomy repair is accomplished in a shorter time as a result of the reduction in time from not lysing a significant number of adhesions, or adhesions of significant size, and the patient is treated with 30% less anesthesia due to the reduced length of the surgery. In addition, the overall risk to the patient resulting from the surgery is significantly reduced due to the reduced number and size of adhesions which need to be lysed, as the lysing of adhesions is traditionally risky due to inhibition of surgeon visibility which raises the risk that a nerve or vessel may be unintentionally cut in the lysing process.

Following the phase of the repair procedure when the two incised sections of the small intestine are sutured back together, and prior to closing of the surgical site, the surgeon places a laparoscopic nozzle approximately 0.1-0.5 inches away from the suture site and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 180 degrees in each direction relative to a starting point to evenly coat the entire surface of the repaired small intestine tissue. The resulting ECM gel has a storage modulus between 2500 and 3000 Pa and evenly coats the surface of the repaired intestinal tissue. The laparoscopic incisions are then closed, and any remaining CO2 is dissolved in the blood and safely eliminated through normal physiological processes. The storage modulus of the ECM gel is less than the elastic modulus of the small intestine and the surrounding tissue (20-40 kPa), and does not impede the biomechanics of digestion, including movement of digestate through the small intestine. The resulting ECM barrier formed over the repaired intestinal tissue operates to permit gliding of the abdominal wall over the small intestine tissue, and permits gliding of the small intestine tissue over other organs it is in contact with; and prevents the formation of post-surgical adhesions. Also as a result of the application of the application of the adhesion barrier, there is a 50% reduction in the formation of scar tissue resulting from the ileostomy repair.

### Example 8: Abdominal Surgery—Colorectal Resection & Ostomy Creation

In this example, the fluid dispersion device and ECM fluid of one or more embodiments are used to produce an adhesion barrier, prevent the formation of adhesions along the small intestine, large intestine, and to improve patient outcomes in a colorectal resection, and an ileostomy creation procedure.

A subject is undergoing a colorectal resection as a treatment for colon cancer. A surgeon is performing colorectal resection to remove the malignant tissue and is diverting the subject's bowel in the large intestine as part of a colostomy creation. The surgeon is performing the surgery using a laparoscopic surgery method to minimize the trauma to the subject resulting from the surgery.

The device comprises a 0.6 mm laparoscopic nozzle, ECM fluid contained within a syringe connected to a constant force spring and dispersed through an annular area of the nozzle. The 0.6 mm laparoscopic nozzle is connected to a compressed CO2 source which disperses compressed CO2 from the center of the nozzle, and aerosolizes the fluid to a high stiffness ECM hydrogel as it is dispersed from the nozzle. The compressed CO2 is pressurized to 5 psig.

The colorectal recession is performed as in known within the art. Following suture of the two sections of healthy colon tissue to one another, the surgeon places a laparoscopic nozzle approximately 0.1-2.0 inches away from the suture site and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 180 degrees around the intestine in each direction relative to a starting point to evenly coat the entire surface of the repaired bowel tissue, and the surrounding tissues. The resulting ECM gel has a storage modulus between 2500 and 3000 Pa and evenly coats the surface of the repaired intestinal tissue. The laparoscopic incisions are then closed, and any remaining CO2 is dissolved in the blood and safely eliminated through normal physiological processes. The storage modulus of the ECM gel is less than the elastic modulus of the large intestine and the surrounding tissue, and does not impede the biomechanics of bowel function, including bowel movements. The resulting ECM barrier formed over the repaired bowel tissue operates to permit gliding of the abdominal wall over the large intestine tissue, and permits gliding of the large intestine tissue over other sections of large intestine tissue it is in contact; and prevents the formation of post-surgical adhesions. Also as a result of the application of the application of the adhesion barrier, there is a 50% reduction in the formation of scar tissue resulting from the colorectal rescission.

In parallel with the colorectal resection, a colostomy creation procedure is performed as is known within the art. In this example, a colostomy procedure is performed by bringing the incised section of the large intestine, the stoma, to the wall abdominal wall and suturing the large intestine in place. At this time, an adhesion barrier is applied to the surface of the incised large intestine tissue, along the sutured section, along the section of tissue extending outward from the body, and along the surface of the tissue remaining within the body, effectively coating the entire section of incised tissue with the ECM barrier.

The surgeon places a dispensing nozzle approximately 0.5-3.0 inches away from the suture site and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 180 degrees in each direction relative to a starting point to evenly coat the entire surface of the incised and sutured small intestinal tissue. The resulting ECM gel has a storage modulus between 2500 and 3000 Pa and evenly coats the surface of the repaired intestinal tissue. The incisions are then closed with the stoma extending outward from the subject, and a colostomy bag is placed over the stoma. Any remaining CO2 is dissolved in the blood and safely eliminated through normal physiological processes. The storage modulus of the ECM gel is less than the elastic modulus of the large intestine and the surrounding tissue, and does not impede the biomechanics of digestion, or bowel movement.

In the months following the surgery, once the section of the large intestine which has been repaired has begun to heal, when inflammation has receded, and once any resulting infections have been controlled or eradicated, the subject report for a colostomy repair procedure.

The colostomy repair procedure is performed as is known within the art. When the surgeon reaches the phase of the repair procedure when it is time lyse adhesions, approximately 80% fewer adhesions are noted to have been formed as a result of colostomy creation, and the few adhesions which have been formed are 50% smaller in size relative the size of intestinal adhesions traditionally resulting from such procedures. The colostomy repair is accomplished in a shorter time as a result of the reduction in time from not lysing a significant number of adhesions, or adhesions of significant size, and the patient is treated with 30% less anesthesia due to the reduced length of the surgery. In addition, the overall risk to the patient resulting from the surgery is significantly reduced due to the reduced number and size of adhesions which need to be lysed, as the lysing of adhesions is traditionally risky due to inhibition of surgeon visibility which raises the risk that a nerve or vessel may be unintentionally cut in the process.

Following the phase of the repair procedure when the two incised sections of the large intestine are sutured back together, and prior to closing of the surgical site, the surgeon places a laparoscopic nozzle approximately 0.1-0.5 inches away from the suture site and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 180 degrees around the sutured intestine in each direction relative to a starting point to evenly coat the entire surface of the repaired large intestine tissue, and the surrounding tissues. The resulting ECM gel has a storage modulus between 2500 and 3000 Pa and evenly coats the surface of the repaired intestinal tissue. The laparoscopic incisions are then closed, and any remaining CO2 is dissolved in the blood and safely eliminated through normal physiological processes. The storage modulus of the ECM gel is less than the elastic modulus of the large intestine and the surrounding tissue, and does not impede the biomechanics of digestion, including movement of digestate through the small intestine. The resulting ECM barrier formed over the repaired intestinal tissue operates to permit gliding of the abdominal wall over the small intestine tissue, and permits gliding of the large intestine tissue over other organs it is in contact with; and prevents the formation of post-surgical adhesions. Also as a result of the application of the application of the adhesion barrier, there is a 50% reduction in the formation of scar tissue resulting from the colostomy repair on the large intestine.

### Example 9: Pelvic Surgery—Cesarean Section

In this example, the fluid dispersion device and ECM fluid of one or more embodiments are used to produce an adhesion barrier, prevent the formation of adhesions on the fallopian tubes and uterus, as to improve patient outcomes in a caesarian section.

The device comprises a 0.6 mm laparoscopic nozzle, ECM fluid contained within a syringe connected to a constant force spring and dispersed through an annular area of the nozzle. The 0.6 mm laparoscopic nozzle is connected to a compressed CO2 source which disperses compressed CO2 from the center of the nozzle, and aerosolizes the fluid to a high stiffness ECM hydrogel as it is dispersed from the nozzle. The compressed CO2 is pressurized to 10 psig.

A subject is undergoing a caesarian section following an extended delivery and upon deceleration of neonate heart rate. A caesarian section is performed as is known within the art, inadvertently resulting in a partial rupture of a fallopian tube. Following removal of the neonate and prior closing of the uterine wall, the surgeon places a dispensing nozzle approximately 0.5-3.0 inches away from the fallopian tubes and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 180 degrees in each direction relative to a starting point to evenly coat the interior of the uterus and the fallopian tube with the adhesion barrier. The resulting ECM gel has a storage modulus between 2500 and 3000 Pa and evenly coats the surface of the repaired uterine tissue. Any remaining CO2 is dissolved in the blood and safely eliminated through normal physiological processes. The storage modulus of the ECM gel is less than the elastic modulus of the uterine wall, fallopian tubes, and the surrounding tissue, and does not impede the biomechanics of ovulation, and menstruation. The surgeon then proceeds with manual closure of the uterine wall via sutures or other methods known within the art.

Following closure of the uterine wall via suture, the surgeon places a dispensing nozzle approximately 3.0-5.0 inches away from the uterine wall and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 130 degrees in an arc across the uterine wall of the subject to evenly coat the surface of the uterine wall with the adhesion barrier. The resulting ECM gel has a storage modulus between 2500 and 3000 Pa and evenly coats the surface of the repaired uterine wall. Any remaining CO2 is dissolved in the blood and safely eliminated through normal physiological processes. The storage modulus of the ECM gel is less than the elastic modulus of the uterine wall the surrounding tissue, and does not impede the biomechanics of movement of the uterus in the peritoneal cavity.

Following the surgery, the subject experiences a 75% reduction in formation of scar tissue along the fallopian tubes and uterus. As a result of the reduction of scar tissue in the fallopian tubes, the subject is able to continue experiencing normal ovulation as eggs descend from the fallopian tube, lowering the risk of infertility as a result of the C-section. As a result of the reduction in scar tissue along the uterine wall, future embryos are less likely to cause abnormal expansion of tissue at the site of the first C-section, thus reducing the risk of uterine wall rupture. Similarly, the reduction of scar tissue along the fallopian tube reduces the risk of ectopic pregnancy. The subject further experiences a 75% reduction in the formation of adhesions between the uterine wall and the peritoneal activity, and a 50% reduction in adhesion size of adhesions. Overall, the subject experiences improved healing, reduced scarring, reduced adhesions, ongoing fertility, and reduced risk of complications in a subsequent pregnancies resulting from the C-section.

### Example 10: Biopsy Collection

In this example, the fluid dispersion device and ECM fluid of one or more embodiments are used to produce an adhesion barrier, and prevent formation of scar tissue at the location of a biopsy, as to improve patient outcomes following the biopsy.

The device comprises a 0.6 mm laparoscopic nozzle, ECM fluid contained within a syringe connected to a constant force spring and dispersed through an annular area of the nozzle. The 0.6 mm laparoscopic nozzle is connected to a compressed CO2 source which disperses compressed CO2 from the center of the nozzle, and aerosolizes the fluid to a high stiffness ECM hydrogel as it is dispersed from the nozzle. The compressed CO2 is pressurized to 10 psig.

A subject is undergoing biopsy collection for analysis to determine the cause of an abnormal skin condition under suspicion of malignancy. The biopsy is performed as is known within the art. Following collection of the sample and closure of the incision site with a suture, or other means known within the art, the surgeon places a dispensing nozzle approximately 3.0-5.0 inches away from the incision site and triggers the valve, resulting in aerosolization of the ECM fluid as it is dispensed from the nozzle and dispersed into a fine mist with an average particle diameter between 100 micrometers to 250 micrometers. The surgeon evenly coats the incision site with the adhesion barrier. The resulting ECM gel has a storage modulus between 2500 and 3000 Pa and evenly coats the surface of the repaired dermal tissue. Following application of the ECM gel adhesion barrier to the surface of the skin, keloid formation is reduced, resulting in a 25% reduction in scar tissue at the incision site as it recovers, and a normalized rate of melanin production at the incision site. Overall, the patient outcome is improved as result of the reduction in formation of scar tissue at the incision site.

### Example 11: Improved Breast Augmentations

In this example, the fluid dispersion device and ECM fluid of one or more embodiments are used to produce an adhesion barrier along a silicone breast implant; and prevent formation of scar tissue, or contracture, as to reduce the risk of implant failure, or to extend implant life.

The device comprises a 0.6 mm laparoscopic nozzle, ECM fluid contained within a syringe connected to a constant force spring and dispersed through an annular area of the nozzle. The 0.6 mm laparoscopic nozzle is connected to a compressed CO2 source which disperses compressed CO2 from the center of the nozzle, and aerosolizes the fluid to a high stiffness ECM hydrogel as it is dispersed from the nozzle. The compressed CO2 is pressurized to 10 psig.

A subject is undergoing breast augmentation surgery with the implantation of silicone breasts implants. The breast augmentation surgery is performed as is known within the art prior to implantation. Prior to implantation, the surgeon places a dispensing nozzle approximately 3.0-5.0 inches away from the implant and triggers the valve, resulting in aerosolization of the ECM fluid as it is dispensed from the nozzle and dispersed into a fine mist with an average particle diameter between 100 micrometers to 250 micrometers. The surgeon rotates the implant 360 degrees on a rotating platform as the ECM gel is aerosolized from the nozzle to evenly coat the surface of the implant with the adhesion barrier.

In parallel, the surgical cavity of the breast, which has been prepared for implantation, is also coated with the adhesion barrier. The surgeon places a dispensing nozzle approximately 0.5-3.0 inches away from the surface of the tissue within the surgical cavity and triggers the valve, resulting in aerosolization of the ECM fluid as it is dispensed from the nozzle and dispersed into a fine mist with an average particle diameter between 100 micrometers to 250 micrometers. The surgeon rotates the nozzle 130 degrees in an arc, multiple times, as the ECM gel is aerosolized from the nozzle to evenly and completely coat the surface of the surgical cavity with the adhesion barrier. The adhesion barrier coated implant is then implanted into the surgical cavity.

The resulting ECM gel has a storage modulus between 2500 and 3000 Pa and evenly coats the surface of the surgical cavity and the surface of the breast implant. The storage modulus of the ECM gel is greater than the elastic modulus of the adipose tissue of the breast and other surrounding tissues (0.5-1.0 kPa), and prevents direct contact between the implant and the surrounding tissues. Because there is essentially no biomechanical function served by the adipose tissue of the breast, the higher stiffness of the ECM gel relative to the low elastic modulus of the adipose tissue does not impede any biological functions.

Following the implant, the subject experiences an 85% reduction of scar tissue within the surgical cavity surrounding the breast implant. Further, the subject does not experience a contracture, a physical encapsulation of the implant by the body resulting from the formation of fibrous scar tissue around the implant. As a result of the subject not experiencing formation of excessive scar tissue surrounding the implant, no excess pressure is placed on the implant relative to the initial physiological pressure placed on the implant. The subject does not experience squeezing of the implant, does not experience a spherical or hard implant resulting from excess pressure placed on the implant, and experiences a reduced risk of implant leakage or rupture.

### Example 12: Cardiac Surgery

In this example, the fluid dispersion device and ECM fluid of one or more embodiments are used to produce an adhesion barrier in the thoracic cavity following installation of a pulmonary valve used to correct a congenital heart defect.

The device comprises a 0.6 mm laparoscopic nozzle, ECM fluid contained within a syringe connected to a constant force spring and dispersed through an annular area of the nozzle. The 0.6 mm laparoscopic nozzle is connected to a compressed CO2 source which disperses compressed CO2 from the center of the nozzle, and aerosolizes the fluid to a high stiffness ECM hydrogel as it is dispersed from the nozzle. The compressed CO2 is pressurized to 10 psig.

A 12 year old subject suffering from a malformed pulmonary valve requires a pulmonary valve replacement. The subject was born with a malformed pulmonary valve as a congenital heart defect, and previously had the valve replaced prior to 1 year of age. The pulmonary valve replacement surgery is performed as is known within the art. Prior to closure the thoracic cavity, the surgeon places a dispensing nozzle approximately 1.0-3.0 inches away from the tissue and triggers the valve, resulting in aerosolization of the ECM fluid as it is dispensed from the nozzle and dispersed into a fine mist with an average particle diameter between 100 micrometers to 250 micrometers. The surgeon moves the nozzle approximately 180 degrees in each direction relative to a starting point to evenly coat the interior of the thoracic cavity with the adhesion barrier. The resulting ECM gel has a storage modulus between 2500 and 3000 Pa and evenly coats the surface of the thoracic cavity. Any remaining CO2 is dissolved in the blood and safely eliminated through normal physiological processes. The storage modulus of the ECM gel is less than the elastic modulus of the heart muscles, arteries, and veins, and other the surrounding tissue, and does not impede the biomechanics of ventricular contractions and blood flow through the pulmonary value. The surgeon may then proceed with closure of the thoracic cavity as is known within the art.

Following the pulmonary valve replacement, the subject experiences a reduction in formation of scar tissue and adhesions in the thoracic cavity, reduced formation of adhesions in the thoracic cavity, increased cardiovascular capacity, and an improved patient outcome.

When the subject needs a subsequent pulmonary valve replacement following puberty, the pulmonary valve replacement surgery is performed as is known within the art. However, when performing the subsequent pulmonary valve replacement surgery the surgeon, the surgery is able to proceed more quickly and at lower risk as a result of the reduction in adhesions in the thoracic cavity and scar tissue along the cardiac muscle resulting from the prior surgery.

### Example 13: Cardiac Surgery

In this example, the fluid dispersion device and ECM fluid of one or more embodiments are used to produce an adhesion barrier in the thoracic cavity following installation of a left ventricular assist device (LVAD).

The device comprises a 0.6 mm laparoscopic nozzle, ECM fluid contained within a syringe connected to a constant force spring and dispersed through an annular area of the nozzle. The 0.6 mm laparoscopic nozzle is connected to a compressed CO2 source which disperses compressed CO2 from the center of the nozzle, and aerosolizes the fluid to a high stiffness ECM hydrogel as it is dispersed from the nozzle. The compressed CO2 is pressurized to 5 psig.

The subject is prepared for installation of a LVAD, and installation of the device is performed as is known within the art. Prior to closure the thoracic cavity, the surgeon places a dispensing nozzle approximately 1.0-3.0 inches away from the cardiac tissue and triggers the valve, resulting in aerosolization of the ECM fluid as it is dispensed from the nozzle and dispersed into a fine mist with an average particle diameter between 100 micrometers to 250 micrometers. The surgeon moves the nozzle in a 130 degrees arc relative to a starting point to evenly coat the interior of the thoracic cavity with the adhesion barrier. The resulting ECM gel has a storage modulus between 2500 and 3000 Pa and evenly coats the surface of the thoracic cavity. Any remaining CO2 is dissolved in the blood and safely eliminated through normal physiological processes. The storage modulus of the ECM gel is less than the elastic modulus of the heart muscles, arteries, and veins, and other the surrounding tissue, and does not impede the biomechanics of ventricular contractions and blood flow through the left ventricle. The surgeon may then proceed with closure of the thoracic cavity as is known within the art.

Following the installation of the LVAD, the subject experiences a reduction in formation of scar tissue, reduced formation of adhesions in the thoracic cavity, increased cardiovascular capacity, and an improved patient outcome.

Following installation of the LVAD, the subject later requires a heart transplant. The subject is prepared for a heart transplant as is known within the art. Prior to transplant of the donor heart into the subject, the surgeon must prepare the thoracic cavity for the donor heart by removing the subject's failing heart. Due to application of the ECM gel-based adhesion barrier, there is a significant reduction formation of scar tissue surrounding the LVAD. The surgeon removes approximately 50% less scar tissue surrounding the LVAD in order to access the heart than generally would have been present in the absence of application of the adhesion barrier. As a result of the significant reduction in scar tissue development, the surgeon is required to expend significantly less time in removing the scar tissue while the donor heart remains on ice. Similarly, there is an 85% reduction in formation of adhesions in the thoracic cavity resulting from the prior surgery, and the surgeon is required to expend significantly less time lysing adhesions when removing the subject's failing heart. Due to the donor heart remaining on ice for shortened period while the scar tissue is removed and adhesions are lysed, there is remarkably reduced risk of the donor heart failing to regain full function, resulting in a decreased risk of patient death.

What is claimed is:

1. A device for delivering a multi-component fluid, the device comprising:
a) a tube having a distal end and a proximal end, wherein the tube comprising a first lumen, a second lumen, and a dispersant lumen, each lumen extending from the proximal end to the distal end of the tube, wherein:
(i) the first lumen is configured to receive a first component of the multi-component fluid,
(ii) the second lumen is configured to receive a second component of the multi-component fluid, and
(iii) the dispersant lumen is configured to receive a dispersant fluid;
b) a mixer coupled to the distal end of the tube, the mixer comprising a chamber disposed within a housing and a mixer body disposed within the chamber, wherein a proximal end of the chamber is in fluid communication with the first lumen and with the second lumen to receive the first component and the second component within the chamber and mix the first component and the second component using the mixer body to form the multi-component fluid, wherein the mixer body comprises a dispersant passageway therein that extends from a proximal end of the mixer body to a distal end of the mixer body and is in fluid communication with the dispersant lumen to receive the dispersant fluid therefrom, so as to deliver the dispersant fluid to a distal end of the chamber or to a location distal to the distal end of the chamber; and
c) a nozzle disposed distal to the mixer body and/or distal to the chamber, wherein the nozzle comprises a nozzle inlet, a nozzle body, and a nozzle outlet, wherein the nozzle receives the multi-component fluid from the chamber and the dispersant from the dispersant passageway, so as to deliver the multi-component fluid and the dispersant through the nozzle outlet.

2. The device of claim 1, wherein the dispersant passageway extends along a central axis of the mixer body from the mixer proximal end to the mixer distal end.

3. The device of claim 1, wherein the dispersant passageway is proximally coupled to the dispersant lumen.

4. The device of claim 1, wherein the first lumen is in fluid communication with a first container, such that the first lumen is configured to receive the first component of the multi-component fluid from the first container

5-127. (canceled)

128. A system comprising:
a) device for delivering a multicomponent fluid, the device comprising:
i) a tube having a distal end and a proximal end, wherein the tube comprising a first lumen, a second lumen, and a dispersant lumen, each lumen extending from the proximal end to the distal end of the tube, wherein:
(1) the first lumen is configured to receive a first component of the multi-component fluid,
(2) the second lumen is configured to receive a second component of the multi-component fluid, and
(3) the dispersant lumen is configured to receive a dispersant fluid;
ii) a mixer coupled to the distal end of the tube, the mixer comprising a chamber disposed within a housing and a mixer body disposed within the chamber, wherein a proximal end of the chamber is in fluid communication with the first lumen and with the second lumen to receive the first component and the second component within the chamber and mix the first component and the second component using the mixer body to form the multi-component fluid, wherein the mixer body comprises a dispersant passageway therein that extends from a proximal end of the mixer body to a distal end of the mixer body and is in fluid communication with the dispersant lumen to receive the dispersant fluid therefrom, so as to deliver the dispersant fluid to a distal end of the chamber or to a location distal to the distal end of the chamber;
iii) a nozzle disposed distal to the mixer body and/or distal to the chamber, wherein the nozzle comprises a nozzle inlet, a nozzle body, and a nozzle outlet, wherein the nozzle receives the multi-component fluid from the chamber and the dispersant from the dispersant passageway, so as to deliver the multi-component fluid and the dispersant through the nozzle outlet;
iv) a dispersant nozzle coupled to a distal end of the mixer body, wherein the dispersant nozzle comprises a dispersant nozzle outlet, wherein the dispersant nozzle is in fluid communication with the dispersant passageway; and
b) a multi component fluid in fluidic communication with the device, the multi component fluid comprising:
i) an extracellular (ECM) matrix;
ii) a buffering solution, and
c) a pressurized dispersant fluid in fluidic communication with the device.

129. The system of claim 128, wherein the pressurized dispersant fluid comprises CO2.

130. The system of claim 129, wherein the buffering solution comprises phosphate-buffered saline.

131. The system of claim 130, wherein the dispersant nozzle outlet is between 0.3 and 0.9 mm in diameter.

132. (canceled)

133. The system of claim 132, wherein the dispersant fluid aerosolizes the multi-component fluid upon delivery from the nozzle outlet into an ECM hydrogel mist comprising particles.

134. The system of claim 133, wherein the particles comprise an average particle diameter from about 7 um to about 300 um.

135. (canceled)

136. (canceled)

137. (canceled)

138. (canceled)

139. (canceled)

140. (canceled)

141. The system of claim 140, wherein the multi-component is buffered to a pH from about 6.5 to about 7.0.

142. The system of claim 128, wherein the multicomponent fluid comprises an ECM hydrogel scaffold.

143. The system of claim 128, wherein the extracellular (ECM) matrix, the buffering solution, and the pressurized dispersant fluid collectively comprise an ECM hydrogel scaffold.

144. The system of claim 143, wherein the ECM hydrogel scaffold comprises a storage modulus of at least 1000 Pa.

145. (canceled)

146. (canceled)

147. (canceled)

148. (canceled)

149. (canceled)

150. The system of claim 133, wherein the particles comprise droplets.

151. (canceled)

152. (canceled)

153. The system of claim 133, wherein the ECM comprise a pH of about 1 to about 3.

154. (canceled)

155. (canceled)

156. The system of claim 133, wherein the ECM, the multicomponent fluid, or both, is a shear thinning fluid.

157. (canceled)

158. (canceled)

159. (canceled)

160. (canceled)

161. A device for delivering a fluid, the device comprising:
a) a tube having a distal end and a proximal end, wherein the tube comprises a plurality of lumens, wherein:
(i) a first lumen of the plurality of lumens is configured to receive a first fluid, and
(ii) a second lumen of the plurality of lumens is configured to receive a second fluid;
b) a mixer coupled to the tube, the mixer comprising a chamber is in fluid communication at least one lumen to receive the first fluid, and a dispersant passageway therein that extends through the mixer and which is in fluid communication with the second lumen to receive the second fluid; and
c) a nozzle disposed distal to the chamber, wherein the nozzle receives the first fluid from the mixer and the second fluid from the dispersant passageway, so as to deliver the first and second fluids through a nozzle outlet.

162. The device of claim 161, further comprising: a third lumen of the plurality of lumens configured to receive a third fluid.

163. (canceled)

164. (canceled)

165. The device of claim 163, wherein the nozzle is configured to disperse the mixture with the second fluid.
